SEQUENCE LISTING (110 sequences)

SEQ ID NO: 1 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 6 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 7 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 8 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 9 - protein, 5 aa, Artificial Sequence
  FAP(4B9)  CDR-H1, synthesized
SYAMS

SEQ ID NO: 10 - protein, 17 aa, Artificial Sequence
  FAP(4B9)  CDR-H2, synthesized
AIIGSGASTYYADSVKG

SEQ ID NO: 11 - protein, 8 aa, Artificial Sequence
  FAP(4B9)  CDR-H3, synthesized
GWFGGFNY

SEQ ID NO: 12 - protein, 12 aa, Artificial Sequence
  FAP(4B9)  CDR-L1, synthesized
RASQSVSRSYLA

SEQ ID NO: 13 - protein, 7 aa, Artificial Sequence
  FAP(4B9)  CDR-L2, synthesized
VGSRRAT

SEQ ID NO: 14 - protein, 9 aa, Artificial Sequence
  FAP(4B9)  CDR-L3, synthesized
QQGIMLPPT

SEQ ID NO: 15 - protein, 5 aa, Artificial Sequence
  FAP(28H1)  CDR-H1, synthesized
SHAMS

SEQ ID NO: 16 - protein, 16 aa, Artificial Sequence
  FAP(28H1)  CDR-H2, synthesized
AIWASGEQYYADSVKG

SEQ ID NO: 17 - protein, 8 aa, Artificial Sequence
  FAP(28H1)  CDR-H3, synthesized
GWLGNFDY

SEQ ID NO: 18 - protein, 12 aa, Artificial Sequence
  FAP(28H1)  CDR-L1, synthesized
RASQSVSRSYLA

SEQ ID NO: 19 - protein, 7 aa, Artificial Sequence
  FAP(28H1)  CDR-L2, synthesized
GASTRAT

SEQ ID NO: 20 - protein, 9 aa, Artificial Sequence
  FAP(28H1)  CDR-L3, synthesized
QQGQVIPPT

SEQ ID NO: 21 - protein, 117 aa, Artificial Sequence
  FAP(4B9)  VH, synthesized
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 22 - protein, 108 aa, Artificial Sequence
  FAP(4B9)  VL, synthesized
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 23 - protein, 116 aa, Artificial Sequence
  FAP(28H1) VH, synthesized
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 24 - protein, 108 aa, Artificial Sequence
  FAP(28H1) VL, synthesized
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 25 - protein, 16 aa, Artificial Sequence
  CD19 (8B8-2B11) CDR-L1, synthesized
KSSQSLETSTGTTYLN

SEQ ID NO: 26 - protein, 7 aa, Artificial Sequence
  CD19 (8B8-2B11) CDR-L2, synthesized
RVSKRFS

SEQ ID NO: 27 - protein, 9 aa, Artificial Sequence
  CD19 (8B8-2B11) CDR-L3, synthesized
LQLLEDPYT

SEQ ID NO: 28 - protein, 5 aa, Artificial Sequence
  CD19 (8B8-2B11) CDR-H1, synthesized
DYIMH

SEQ ID NO: 29 - protein, 17 aa, Artificial Sequence
  CD19 (8B8-2B11) CDR-H2, synthesized
YINPYNDGSKYTEKFQG

SEQ ID NO: 30 - protein, 12 aa, Artificial Sequence
  CD19 (8B8-2B11) CDR-H3, synthesized
GTYYYGPQLFDY

SEQ ID NO: 31 - protein, 5 aa, Artificial Sequence
  CD19 (8B8-018) CDR-H1, synthesized
DYIMH

SEQ ID NO: 32 - protein, 17 aa, Artificial Sequence
  CD19 (8B8-018) CDR-H2, synthesized
YINPYNDGSKYTEKFQG

SEQ ID NO: 33 - protein, 12 aa, Artificial Sequence
  CD19 (8B8-018) CDR-H3, synthesized
GTYYYGSALFDY

SEQ ID NO: 34 - protein, 16 aa, Artificial Sequence
  CD19 (8B8-018) CDR-L1, synthesized
KSSQSLENPNGNTYLN

SEQ ID NO: 35 - protein, 7 aa, Artificial Sequence
  CD19 (8B8-018) CDR-L2, synthesized
RVSKRFS

SEQ ID NO: 36 - protein, 9 aa, Artificial Sequence
  CD19 (8B8-018) CDR-L3, synthesized
LQLTHVPYT

SEQ ID NO: 37 - protein, 121 aa, Artificial Sequence
  CD19 (8B8-2B11) VH, synthesized
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSS

SEQ ID NO: 38 - protein, 112 aa, Artificial Sequence
  CD19 (8B8-2B11) VL, synthesized
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 39 - protein, 121 aa, Artificial Sequence
  CD19 (8B8-018) VH, synthesized
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGSALFDYWGQGTTVTVSS

SEQ ID NO: 40 - protein, 112 aa, Artificial Sequence
  CD19 (8B8-018) VL, synthesized
DIVMTQTPLSLSVTPGQPASISCKSSQSLENPNGNTYLNWYLQKPGQSPQLLIYRVSKRFSGVPDRFSGSGSGTDFTLKISRVEAEDVGVYYCLQLTHVPYTFGQGTKLEIK

SEQ ID NO: 41 - protein, 5 aa, Artificial Sequence
  Peptide linker G4S, synthesized
GGGGS

SEQ ID NO: 42 - protein, 10 aa, Artificial Sequence
  Peptide linker (G4S)2, synthesized
GGGGSGGGGS

SEQ ID NO: 43 - protein, 10 aa, Artificial Sequence
  Peptide linker (SG4)2, synthesized
SGGGGSGGGG

SEQ ID NO: 44 - protein, 17 aa, Artificial Sequence
  Peptide linker particular, synthesized
GGGGGSGGGGSSGGGGS

SEQ ID NO: 45 - protein, 15 aa, Artificial Sequence
  Peptide linker (G4S)3, synthesized
GGGGSGGGGSGGGGS

SEQ ID NO: 46 - protein, 14 aa, Artificial Sequence
  Peptide linker G4(SG4)2, synthesized
GGGGSGGGGSGGGG

SEQ ID NO: 47 - protein, 20 aa, Artificial Sequence
  Peptide linker (G4S)4, synthesized
GGGGSGGGGSGGGGSGGGGS

SEQ ID NO: 48 - protein, 10 aa, Artificial Sequence
  Peptide linker GSPGSSSSGS, synthesized
GSPGSSSSGS

SEQ ID NO: 49 - protein, 8 aa, Artificial Sequence
  Peptide linker GSGSGSGS, synthesized
GSGSGSGS

SEQ ID NO: 50 - protein, 8 aa, Artificial Sequence
  Peptide linker GSGSGNGS, synthesized
GSGSGNGS

SEQ ID NO: 51 - protein, 8 aa, Artificial Sequence
  Peptide linker GGSGSGSG, synthesized
GGSGSGSG

SEQ ID NO: 52 - protein, 6 aa, Artificial Sequence
  Peptide linker GGSGSG, synthesized
GGSGSG

SEQ ID NO: 53 - protein, 4 aa, Artificial Sequence
  Peptide linker GGSG, synthesized
GGSG

SEQ ID NO: 54 - protein, 8 aa, Artificial Sequence
  GGSGNGSG, synthesized
GGSGNGSG

SEQ ID NO: 55 - protein, 8 aa, Artificial Sequence
  Peptide inker GGNGSGSG, synthesized
GGNGSGSG

SEQ ID NO: 56 - protein, 6 aa, Artificial Sequence
  Peptide linker GGNGSG, synthesized
GGNGSG

SEQ ID NO: 57 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 58 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 59 - protein, 556 aa, Homo sapiens [sequence omitted]

SEQ ID NO: 60 - protein, 107 aa, Artificial Sequence
  CH2 domain, synthesized
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 61 - protein, 106 aa, Artificial Sequence [sequence omitted]

SEQ ID NO: 62 - protein, 98 aa, Artificial Sequence
  CH1 domain, synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 63 - protein, 10 aa, Artificial Sequence
  Hinge region with X being S or P, synthesized
  misc_feature (8)..(8): Xaa can be any naturally occurring amino acid
DKTHTCPXCP

SEQ ID NO: 64 - protein, 7 aa, Artificial Sequence
  shorter hinge region  with X being S or P, synthesized
  misc_feature (5)..(5): Xaa can be any naturally occurring amino acid
HTCPXCP

SEQ ID NO: 65 - protein, 5 aa, Artificial Sequence
  shortest hinge region with X being S or P, synthesized
  misc_feature (3)..(3): Xaa can be any naturally occurring amino acid
CPXCP

SEQ ID NO: 66 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 67 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 68 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 69 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 70 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 71 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 72 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 73 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 74 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 75 - protein, 281 aa, Homo sapiens [sequence omitted]

SEQ ID NO: 76 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 77 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 78 - protein, 250 aa, Homo sapiens [sequence omitted]

SEQ ID NO: 79 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 80 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 81 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 82 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 83 - protein, 391 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 84 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 85 - protein, 790 aa, Artificial Sequence
  first fusion polypeptide (P1AA1199), synthesized
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 86 - protein, 446 aa, Artificial Sequence
  second fusion polypeptide (P1AA1199), synthesized
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 215 aa, Artificial Sequence
  light chain (P1AA1199, P1AA1235), synthesized
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 602 aa, Artificial Sequence
  first fusion polypeptide (P1AA1235), synthesized
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 89 - protein, 634 aa, Artificial Sequence
  second fusion polypeptide (P1AA1235, P1AA1259), synthesized
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 90 - protein, 608 aa, Artificial Sequence
  first fusion polypeptide (P1AA1259), synthesized
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 91 - protein, 672 aa, Artificial Sequence
  second fusion polypeptide (P1AA9626) without linker, synthesized
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 682 aa, Artificial Sequence
  second fusion polypeptide (with (G4S)2 linker), synthesized
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 710 aa, Artificial Sequence
  Dimeric hu 4-1BBL (71-248) - CL* Fc knob chain (construct 2.4), synthesized
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 291 aa, Artificial Sequence
  Monomeric hu 4-1BBL (71-248) - CH1* (construct 2.4), synthesized
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 95 - protein, 447 aa, Artificial Sequence
  anti-FAP (4B9) Fc hole chain (construct 2.4), synthesized
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 215 aa, Artificial Sequence; identical to SEQ ID NO: 87
  anti-FAP (4B9) light chain (construct 2.4), synthesized

SEQ ID NO: 97 - protein, 445 aa, Artificial Sequence
  DP47 Fc hole chain, synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 215 aa, Artificial Sequence
  DP47 light chain, synthesized
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 638 aa, Artificial Sequence
  second fusion polypeptide of P1AA1233, synthesized
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 100 - protein, 219 aa, Artificial Sequence
  CD19 (2B11) light chain, synthesized
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 638 aa, Artificial Sequence; identical to SEQ ID NO: 99
  second fusion polypeptide of P1AA1258, synthesized

SEQ ID NO: 102 - protein, 451 aa, Artificial Sequence
  second fusion polypeptide of P1AA10776, synthesized
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 451 aa, Artificial Sequence
  anti-CD19(8B8-2B11) Fc hole chain, synthesized
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 104 - protein, 219 aa, Artificial Sequence; identical to SEQ ID NO: 100
  anti-CD19(8B8-2B11) light chain, synthesized

SEQ ID NO: 105 - protein, 5 aa, Artificial Sequence
  CH1 connector, synthesized
EPKSC

SEQ ID NO: 106 - protein, 330 aa, Homo sapiens [sequence omitted]

SEQ ID NO: 107 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 109 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 110 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2020/0347115 A1
(43) Pub. Date: Nov. 5, 2020

(54) NOVEL TNF FAMILY LIGAND TRIMER-CONTAINING ANTIGEN BINDING MOLECULES

(71) Applicant: Hoffmann-La Roche Inc., Little Falls, NJ (US)

(72) Inventors: Harald Duerr, Penzberg (DE); Claudia Ferrara Koller, Schlieren (CH); Guy Georges, Penzberg (DE); Friederike Hesse, Penzberg (DE); Sabine Imhof-Jung, Penzberg (DE); Christina Claus, Schlieren (CH); Wei Xu, Schlieren (CH)

(73) Assignee: Hoffmann-La Roche Inc., Little Falls, NJ (US)

(21) Appl. No.: 16/861,801

(22) Filed: Apr. 29, 2020

Related U.S. Application Data
(63) Continuation of application No. PCT/EP2018/079784, filed on Oct. 31, 2018.

(30) Foreign Application Priority Data
Nov. 1, 2017 (EP) 17199593.9

(51) Int. Cl. C07K 14/705; C07K 16/28; C07K 16/40

(52) U.S. Cl. CPC C07K 14/70575; C07K 16/2878; C07K 16/2803; C07K 2317/53; C07K 2317/565; C07K 2317/524; C07K 2317/526; C07K 16/40

(57) ABSTRACT

The invention relates to novel TNF family ligand trimer-containing antigen binding molecules comprising two different fusion polypeptides that comprise a spacer domain, an antigen binding domain and three ectodomains of a TNF ligand member or fragments thereof, wherein two of said ectodomains are separated from each other by a spacer domain comprising at least 25 amino acids and wherein the two fusion polypeptides are covalently associated to each other in the spacer domain

### CROSS REFERENCE TO RELATED APPLICATIONS

This application is a continuation of International Application No. PCT/EP2018/079784, filed Oct. 31, 2018, claiming priority to European Application No. 17199593.9, filed, Nov. 1, 2017, which are incorporated herein by reference in its entirety.

### SEQUENCE LISTING

This application contains a Sequence Listing, which has been submitted electronically in ASCII format and is hereby incorporated by reference in its entirety. Said ASCII copy, created on Mar. 30, 2020, and is named Sequence_listing.txt and is 192,749 bytes in size.

### FIELD OF THE INVENTION

The invention relates to novel TNF family ligand trimer-containing antigen binding molecules comprising two different fusion polypeptides that comprise a spacer domain, an antigen binding domain and three ectodomains of a TNF ligand member or fragments thereof, wherein two of said ectodomains are separated from each other by a spacer domain comprising at least 25 amino acids and wherein the two fusion polypeptides are covalently associated to each other in the spacer domain. Some of the TNF family ligand trimer-containing antigen binding molecules may additionally comprise a light chain. The invention further relates to methods of producing these molecules and to methods of using the same.

### BACKGROUND

Ligands interacting with molecules of the TNF (tumor necrosis factor) receptor superfamily have pivotal roles in the organization and function of the immune system. While regulating normal functions such as immune responses, hematopoiesis and morphogenesis, the TNF family ligands (also called cytokines) play a role in tumorigenesis, transplant rejection, septic shock, viral replication, bone resorption, rheumatoid arthritis and diabetes (Aggarwal, Nat. Rev. Immunol. 2003, 3(9), 745-56). The TNF ligand family comprises 18 genes encoding 19 type II (i.e. intracellular N-terminus and extracellular C-terminus) transmembrane proteins, characterized by the presence of a conserved C-terminal domain coined the ‘TNF homology domain’ (THD). This domain is responsible for receptor binding and is thus critical for the biological activity of the TNF ligand family members. The sequence identity between family members is ˜20-30% (Bodmer et al., Trends in Biochemical Sciences 2002, 27(1), 19-26). Members of the TNF ligand family exert their biological function as self-assembling, noncovalent trimers (Banner et al., Cell 1993, 73, 431-445). Thus, the TNF family ligands form a trimer that is able to bind to and to activate the corresponding receptors of TNFR superfamily.

4-1BB (CD137), a member of the TNF receptor superfamily, has been first identified as a molecule whose expression is induced by T-cell activation (Kwon and Weissman, Proc Natl Acad Sci USA 1989, 86, 1963-1967). Subsequent studies demonstrated expression of 4-1BB in T- and B-lymphocytes (Snell et al., Immunol Rev 2011, 244, 197-217; Zhang et al., J Immunol 2010, 184, 787-795), NK-cells (Lin et al., Blood 2008, 112, 699-707), NKT-cells (Kim et al., J Immunol 2008, 180, 2062-2068), monocytes (Kienzle and von Kempis, Int Immunol 2000, 12, 73-82; Schwarz et al., Blood 1995, 85, 1043-1052), neutrophils (Heinisch et al., Eur J Immunol 2000, 30, 3441-3446), mast (Nishimoto et al., Blood 2005, 106, 4241-4248) and dendritic cells as well as cells of non-hematopoietic origin such as endothelial and smooth muscle cells (Broil et al., Am J Clin Pathol 2001, 115, 543-549; Olofsson et al., Circulation 2008, 117, 1292-1301). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., J Immunol 2002, 168, 3755-3762; von Kempis et al., Osteoarthritis Cartilage 1997, 5, 394-406; Zhang et al., J Immunol 2010, 184, 787-795). CD137 signaling is known to stimulate IFNγ secretion and proliferation of NK cells (Buechele et al., Eur J Immunol 2012, 42, 737-748; Lin et al., Blood 2008, 112, 699-707; Melero et al., Cell Immunol 2008, 190, 167-172) as well as to promote DC activation as indicated by their increased survival and capacity to secret cytokines and upregulate co-stimulatory molecules (Choi et al., J Immunol 2009, 182, 4107-4115; Futagawa et al., Int Immunol 2002, 14, 275-286; Wilcox et al., J Immunol 2002, 168, 4262-4267). However, CD137 is best characterized as a co-stimulatory molecule which modulates TCR-induced activation in both the CD4+ and CD8+ subsets of T-cells. In combination with TCR triggering, 4-1BB agonists (agonistic 4-1BB-specific antibodies) have been shown to enhance proliferation of T-cells, stimulate lymphokine secretion and decrease sensitivity of T-lymphocytes to activation-induced cells death (reviewed in Snell et al., Immunol Rev 2011, 244, 197-217).

Expression of 4-1BB ligand (4-1BBL or CD137L) is more restricted and is observed on professional antigen presenting cells (APC) such as B-cells, dendritic cells (DCs) and macrophages. Inducible expression of 4-1BB is characteristic for T-cells, including both αβ and γδ T-cell subsets, and endothelial cells (reviewed in Shao and Schwarz, J Leukoc Biol 2011, 89, 21-29). In addition to their direct effects on different lymphocyte subsets, 4-1BB agonists can also induce infiltration and retention of activated T-cells in the tumor through 4-1BB-mediated upregulation of intercellular adhesion molecule 1 (ICAM1) and vascular cell adhesion molecule 1 (VCAM1) on tumor vascular endothelium (Palazon et al., Cancer Res 2011, 71, 801-811). 4-1BB triggering may also reverse the state of T-cell anergy induced by exposure to soluble antigen that may contribute to disruption of immunological tolerance in the tumor micro-environment or during chronic infections (Wilcox et al., Blood 2004, 103, 177-184).

The available pre-clinical and clinical data clearly demonstrate that there is a high clinical need for effective 4-1BB agonists. However, new generation drug candidates should not only effectively engage 4-1BB on the surface of hematopoietic and endothelial cells but also be capable of achieving that through mechanisms other than binding to Fc-receptors in order to avoid uncontrollable side effects. The latter may be accomplished through preferential binding to and oligomerization on tumor-specific or tumor-associated moieties.

Fusion proteins composed of one extracellular domain of a 4-1BB ligand and a single chain antibody fragment (Mueller et al., J. Immunother. 2008, 31, 714-722; Hornig et al., J. Immunother. 2012, 35, 418-429). However, these molecules are difficult to produce in a technical scale and have unfavourable pharmacokinetic profiles.

There is thus the need to develop new antigen binding molecules that are composed in a way that enable the stabile forming of a costimulatory TNF ligand trimer and that are sufficiently stable to be pharmaceutically useful.

### SUMMARY OF THE INVENTION

The present invention describes how a trimeric TNF ligand can be efficiently fused to an antibody architecture so that the trimeric ligand is correctly assembled and fully functional. Focusing on an antibody-based architecture is guided by the good pharmacokinetic properties of antibodies in general. The antibody architecture is stable compared to other proteins: their expression is also very robust using different cell lines. Their Fc part interacts with the FcRn receptor and therefore preserves the molecules from rapid elimination through intracellular degradation. The novel constructs are expressable with reasonably good titers and produce a good ratio of the wished product.

In one aspect, the invention provides a TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain of said TNF ligand family member or a fragment thereof is fused either directly or via a second peptide linker to the C-terminus of the spacer domain,
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is fused either directly or via a third peptide linker to the C-terminus of the spacer domain or is present in form of a light chain, and
(c) a third ectodomain of said TNF ligand family member or a fragment thereof that is fused either directly or via a fourth peptide linker to
   either the C-terminus of the second ectodomain of said TNF ligand family member in the first fusion polypeptide or to the C-terminus of the spacer domain in the second fusion polypeptide, or
   in case the second part of the antigen binding domain is fused to the C-terminus of the spacer domain of the second fusion protein, to the C-terminus of the second ectodomain of said TNF ligand family member in the first fusion polypeptide,
wherein the spacer domain of the first fusion polypeptide and the spacer domain of the second fusion polypeptide are associated covalently to each other by a disulfide bond.

In a particular aspect, the invention provides a TNF family ligand trimer-containing antigen binding molecule as defined herein before, wherein the first part of the antigen binding domain comprises an antibody heavy chain variable domain and the second part of the antigen binding domain comprises an antibody light chain variable domain or vice versa. More particularly, the first part of the antigen binding domain is an antibody heavy chain Fab fragment and the second part of the antigen binding domain is an antibody light chain Fab fragment or vice versa. In one aspect, the first part of the antigen binding domain and the second part of the antigen binding domain are associated covalently to each other by a disulfide bond.

As described above, the TNF family ligand trimer-containing antigen binding molecule comprises a first and a second fusion polypeptide, both comprising a spacer domain, wherein the spacer domain of the first fusion polypeptide and the spacer domain of the second fusion polypeptide are associated covalently to each other by a disulfide bond.

In one aspect of the invention, the spacer domain comprises an antibody hinge region or a (C-terminal) fragment thereof and an antibody CH2 domain or a (N-terminal) fragment thereof. In another aspect, the spacer domain comprises an antibody hinge region or a fragment thereof, an antibody CH2 domain, and an antibody CH3 domain or a fragment thereof. In a further aspect of the invention, the spacer domain of the first fusion polypeptide and the spacer domain of the second fusion polypeptide comprise modifications promoting the association of the first and second fusion polypeptide. In a particular aspect, the spacer domain of the first fusion polypeptide comprises holes and the spacer domain of the second fusion polypeptide comprises knobs according to the knob into hole method. In a further aspect, the invention comprises a TNF family ligand trimer-containing antigen binding molecule, wherein the spacer domain comprises an antibody hinge region or a fragment thereof and an IgG1 Fc domain. Particularly, the IgG1 Fc domain comprises one or more amino acid substitution that reduces binding to an Fc receptor, in particular towards Fcγ receptor. More particularly, the IgG1 Fc domain comprises the amino acid substitutions L234A, L235A and P329G (numbering according to Kabat EU index).

In a further aspect of the invention, the TNF family ligand is one that costimulates human T-cell activation. Thus, the invention relates to a TNF family ligand trimer-containing antigen binding molecule that costimulates human T-cell activation. In a particular aspect of the invention, the TNF family ligand is 4-1BBL.

In one aspect of the invention, the ectodomain of the TNF ligand family member thus comprises the amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8, particularly the amino acid sequence of SEQ ID NO: 1 or SEQ ID NO: 5. In a particular aspect, the ectodomain of the TNF ligand family member (4-1BBL) comprises the amino acid sequence of SEQ ID NO: 5. The TNF family ligand trimer-containing antigen binding molecule comprises three ectodomains of the TNF ligand family member, and in particular, all three ectodomains of the TNF ligand family member comprise the same amino acid sequence.

The TNF family ligand trimer-containing antigen binding molecule of the invention further comprises an antigen binding domain consisting of a first and second part. In one aspect, the antigen binding domain is capable of specific binding to a tumor associated antigen. In a further aspect, the antigen binding domain is capable of specific binding to Fibroblast Activation Protein (FAP) or CD19.

In one aspect, the antigen binding domain is capable of specific binding to FAP. Particularly, the antigen binding domain capable of specific binding to FAP comprises

(a) a heavy chain variable region (V_{H}FAP) comprising (i) CDR-H1 comprising the amino acid sequence of SEQ ID NO: 9, (ii) CDR-H2 comprising the amino acid sequence of SEQ ID NO: 10, and (iii) CDR-H3 comprising the amino acid sequence of SEQ ID NO: 11, and a light chain variable region (V_{L}FAP) comprising (iv) CDR-L1 comprising the amino acid sequence of SEQ ID NO: 12, (v) CDR-L2 comprising the amino acid sequence of SEQ ID NO: 13, and (vi) CDR-L3 comprising the amino acid sequence of SEQ ID NO: 14, or
(b) a heavy chain variable region (V_{H}FAP) comprising (i) CDR-H1 comprising the amino acid sequence of SEQ ID NO: 15, (ii) CDR-H2 comprising the amino acid sequence of SEQ ID NO: 16, and (iii) CDR-H3 comprising the amino acid sequence of SEQ ID NO: 17, and a a light chain variable region (V_{L}FAP) comprising (iv) CDR-L1 comprising the amino acid sequence of SEQ ID NO: 18, (v) CDR-L2 comprising the amino acid sequence of SEQ ID NO: 19, and (vi) CDR-L3 comprising the amino acid sequence of SEQ ID NO: 20.

In a particular aspect, the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO: 21, and a light chain variable region (V_{L}FAP) comprising an amino acid sequence that is at least about 95%, 96%, 97%, 98%, 99% or 100% identical to the amino acid sequence of SEQ ID NO: 22, or (b) a heavy chain variable region (V_{H}FAP) comprising an amino acid sequence that is at least about 95%, 96%, 97%, 98%, 99% or 100% identical to the amino acid sequence of SEQ ID NO: 23, and a light chain variable region (V_{L}FAP) comprising an amino acid sequence that is at least about 95%, 96%, 97%, 98%, 99% or 100% identical to the amino acid sequence of SEQ ID NO: 24. Particularly, the antigen binding domain capable of specific binding to FAP comprises (a) a heavy chain variable region (V_{H}FAP) comprising the amino acid sequence of SEQ ID NO: 21 and a light chain variable region (V_{L}FAP) comprising an amino acid sequence of SEQ ID NO: 22, or (b) a heavy chain variable region (V_{H}FAP) comprising the amino acid sequence of SEQ ID NO: 23 and a light chain variable region (V_{L}FAP) comprising the amino acid sequence of SEQ ID NO: 24. More particularly, the antigen binding domain capable of specific binding to FAP comprises a heavy chain variable region (V_{H}FAP) comprising the amino acid sequence of SEQ ID NO: 21 and a light chain variable region (V_{L}FAP) comprising an amino acid sequence of SEQ ID NO: 22.

In another aspect, the antigen binding domain is capable of specific binding to CD19. In particular, the antigen binding domain capable of specific binding to CD19 comprises

(a) a heavy chain variable region (V_{H}CD19) comprising (i) CDR-H1 comprising the amino acid sequence of SEQ ID NO: 25, (ii) CDR-H2 comprising the amino acid sequence of SEQ ID NO: 26, and (iii) CDR-H3 comprising the amino acid sequence of SEQ ID NO: 27, and a light chain variable region (V_{L}CD19) comprising (iv) CDR-L1 comprising the amino acid sequence of SEQ ID NO: 28, (v) CDR-L2 comprising the amino acid sequence of SEQ ID NO: 29, and (vi) CDR-L3 comprising the amino acid sequence of SEQ ID NO: 30, or
(b) a heavy chain variable region (V_{H}CD19) comprising (i) CDR-H1 comprising the amino acid sequence of SEQ ID NO: 31, (ii) CDR-H2 comprising the amino acid sequence of SEQ ID NO: 32, and (iii) CDR-H3 comprising the amino acid sequence of SEQ ID NO: 33, and a a light chain variable region (V_{L}CD19) comprising (iv) CDR-L1 comprising the amino acid sequence of SEQ ID NO: 34, (v) CDR-L2 comprising the amino acid sequence of SEQ ID NO: 35, and (vi) CDR-L3 comprising the amino acid sequence of SEQ ID NO: 36.

Particularly, the antigen binding domain capable of specific binding to FAP comprises (a) a heavy chain variable region (V_{H}CD19) comprising an amino acid sequence that is at least about 95%, 96%, 97%, 98%, 99% or 100% identical to the amino acid sequence of SEQ ID NO: 37, and a light chain variable region (V_{L}CD19) comprising an amino acid sequence that is at least about 95%, 96%, 97%, 98%, 99% or 100% identical to the amino acid sequence of SEQ ID NO: 38, or (b) a heavy chain variable region (V_{H}CD19) comprising an amino acid sequence that is at least about 95%, 96%, 97%, 98%, 99% or 100% identical to the amino acid sequence of SEQ ID NO: 39, and a light chain variable region (V_{L}CD19) comprising an amino acid sequence that is at least about 95%, 96%, 97%, 98%, 99% or 100% identical to the amino acid sequence of SEQ ID NO: 40. More particularly, the antigen binding domain capable of specific binding to FAP comprises (a) a heavy chain variable region (V_{H}CD19) comprising the amino acid sequence of SEQ ID NO: 37 and a light chain variable region (V_{L}CD19) comprising the amino acid sequence of SEQ ID NO: 38, or (b) a heavy chain variable region (V_{H}CD19) comprising the amino acid sequence of SEQ ID NO: 39 and a light chain variable region (V_{L}CD19) comprising the amino acid sequence of SEQ ID NO: 40. More particularly, the antigen binding domain capable of specific binding to FAP comprises (a) a heavy chain variable region (V_{H}CD19) comprising the amino acid sequence of SEQ ID NO: 37 and a light chain variable region (V_{L}CD19) comprising the amino acid sequence of SEQ ID NO: 38.

In one aspect, the invention relates to TNF family ligand trimer-containing antigen binding molecule as described herein before, wherein the first, second, third and fourth peptide linker is present and consists of an amino acid sequence selected from the group consisting of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55 and SEQ ID NO: 56. In particular, the peptide linker consists of an amino acid sequence selected from SEQ ID NO: 42 and SEQ ID NO: 44. More particularly, the peptide linker consists of an amino acid sequence of SEQ ID NO: 42.

In another aspect, the invention relates to isolated nucleic acid encoding the TNF family ligand trimer-containing antigen binding molecule as described herein before. The invention further provides a vector, particularly an expression vector, comprising the isolated nucleic acid of the invention or a host cell comprising the isolated nucleic acid or the vector of the invention. In some aspects the host cell is a eukaryotic cell, particularly a mammalian cell.

In another aspect, provided is a method for producing a TNF family ligand trimer-containing antigen binding molecule of the invention, comprising culturing the host cell of the invention under conditions suitable for expression of the antigen binding molecule. In a further aspect, the method further comprises recovering the TNF family ligand trimer-containing antigen binding molecule from the host cell.

The invention further provides a pharmaceutical composition comprising the TNF family ligand trimer-containing antigen binding molecule of the invention and at least one pharmaceutically acceptable excipient.

Also encompassed by the invention is the TNF family ligand trimer-containing antigen binding molecule of the invention, or the pharmaceutical composition of the invention, for use as a medicament. In one aspect is provided the TNF family ligand trimer-containing antigen binding molecule of the invention, or the pharmaceutical composition of the invention, for use in the treatment of a disease in an individual in need thereof. In a specific embodiment, provided is the TNF family ligand trimer-containing antigen binding molecule of the invention, or the pharmaceutical composition of the invention, for use in treating cancer.

Also provided is the use of the TNF family ligand trimer-containing antigen binding molecule of the invention in the manufacture of a medicament for the treatment of a disease in an individual in need thereof, in particular for the manufacture of a medicament for treating cancer, as well as in the manufacture of a medicament for stimulating an immune response.

Provided is furthermore a method of treating an individual having cancer comprising administering to said individual an effective amount of the TNF family ligand trimer-containing antigen binding molecule or the pharmaceutical composition of the invention. The method may further comprise administering an additional therapeutic agent to the individual. In any of the above embodiments the individual is preferably a mammal, particularly a human.

### BRIEF DESCRIPTION OF THE DRAWINGS

FIG. 1A shows a scheme of the FAP (4B9) targeted 4-1BB ligand (71-248) trimer-containing antigen binding molecule P1AA1199 as described in more detail in Example 2.1.

FIG. 1B shows a scheme of the FAP (4B9) targeted 4-1BB ligand (71-248) trimer-containing antigen binding molecule P1AA1235 as described in Example 2.2.

FIG. 1C shows a scheme of the FAP (4B9) targeted 4-1BB ligand (71-248) trimer-containing antigen binding molecule P1AA1259 as described in Example 2.3.

FIG. 1D illustrates the FAP (4B9) targeted 4-1BB ligand (71-248) trimer-containing antigen binding molecule P1AA9626 as described in Example 2.4.

FIG. 1E shows a schematic drawing of the untargeted (DP47 germline) 4-1BB ligand (71-248) trimer-containing antigen binding molecule as described in more detail in Example 2.6. This molecule is used herein as a negative control D.

FIG. 1F shows a schematic drawing of the positive control molecule construct 2.4, i.e. a FAP (4B9) targeted 4-1BB ligand (71-248) trimer-containing antigen binding molecule. Both molecules (control D as well as construct 2.4) are described in more detail in Example 2.6.

FIG. 2A shows the activation of the NFκB signaling pathway by different FAP-targeted 4-1BB ligand trimer-containing Fc(kih) fusion antigen binding molecules. FAP-expressing cell line WM-266-4 was used. Shown are the units of released light (URLs), measured for 0.5 s/well, versus the added concentration in nM of FAP-targeted 4-1BBL ligand trimer-containing antigen binding molecules or control. All URL-values are baseline corrected by subtracting the baseline light emission. All tested FAP-targeted 4-1BBL constructs were able to activate NFκB in a dose-dependent manner as well as FAP-crosslinking dependent. The FAP-targeted 4-1BBL antigen binding molecules P1AA1199, P1AA1259, P1AA1235 and P1AA9626 showed a similar activity as the already described construct 2.4. P1AA1199 showed a slightly lower EC₅₀ value and and a lower plateau than the other tested FAP-targeted 4-1BBL antigen binding molecules. The untargeted 4-1BBL molecule (control D) was not able to induce NFκB-activation and delivered in all settings a baseline. The EC₅₀ values are given in Example 3.

FIG. 2B shows the NFκB activation in the presence of FAP-expressing NIH/3T3-huFAP clone 19 cells. Shown are the units of released light (URLs), measured for 0.5 s/well, versus the added concentration in nM of FAP-targeted 4-1BBL ligand trimer-containing antigen binding molecules or control. All URL-values are baseline corrected by subtracting the baseline light emission. All tested FAP-targeted 4-1BBL constructs were able to activate NFκB in a dose-dependent manner as well as FAP-crosslinking dependent. The FAP-targeted 4-1BBL antigen binding molecules P1AA1199, P1AA1259, P1AA1235 and P1AA9626 showed a similar activity as the already described construct 2.4. P1AA1199 showed a slightly lower EC₅₀ value and and a lower plateau than the other tested FAP-targeted 4-1BBL antigen binding molecules. The untargeted 4-1BBL molecule (control D) was not able to induce NFκB-activation and delivered in all settings a baseline. The EC₅₀ values are given in Example 3.

FIG. 2C shows the NFκB activation in the absence of FAP-expressing cells. Shown are the units of released light (URLs), measured for 0.5 s/well, versus the added concentration in nM of FAP-targeted 4-1BBL ligand trimer-containing antigen binding molecules or control. All URL-values are baseline corrected by subtracting the baseline light emission. All tested FAP-targeted 4-1BBL constructs were able to activate NFκB in a dose-dependent manner as well as FAP-crosslinking dependent. In the absence of FAP-expressing cells (c) only a minor activity could be seen for FAP-targeted 4-1BBL molecules above the baseline of untargeted 4-1BBL (control D). This is due to a minimal FAP-expression by the reporter cells themselves.

FIG. 3A relates to the 4-1BB mediated co-stimulation of sub-optimally TCR triggered TCR PBMCs and hyper-crosslinking by cell surface FAP. It shows the upregulation of surface expressed low affinity IL-2-receptor a chain CD25 as percentage of positive cells in the CD8⁺ T cells. CD25 is upregulated after T cell activation to increase T cell proliferation and survival in the presence of IL-2 and serves as a T cell activation marker.

FIG. 3B relates to the 4-1BB mediated co-stimulation of sub-optimally TCR triggered TCR PBMCs and hyper-crosslinking by cell surface FAP. It shows the upregulation of surface expressed low affinity IL-2-receptor a chain CD25 as percentage of positive cells in the CD4⁺ T cell population. CD25 is upregulated after T cell activation to increase T cell proliferation and survival in the presence of IL-2 and serves as a T cell activation marker.

FIG. 3C shows the expression of 4-1BB (CD137) on the cell surface as percentage of positive cells in the CD8⁺ T cells. All measured values are displayed against the concentration of FAP-targeted 4-1BBL construct 2.4 or untargeted 4-1BBL control D or FAP-targeted 4-1BBL antigen binding molecule of the invention (P1AA1199). P1AA1199 showed similar to the HeLa-human 4-1BB-NFκB-luc reporter cell line assay (see FIG. 2A, FIG. 2B, and FIG. 2C) for same measured parameters the tendency to display a lower EC₅₀ value (CD25 expression) or a lower plateau in the displayed curve (CD137 (4-1BB) expression) compared to construct 2.4. Shown are the mean+/−SD of three technical replicates of each measured point.

FIG. 3D shows the expression of 4-1BB (CD137) on the cell surface as percentage of positive cells in the CD4⁺ T cells. All measured values are displayed against the concentration of FAP-targeted 4-1BBL construct 2.4 or untargeted 4-1BBL control D or FAP-targeted 4-1BBL antigen binding molecule of the invention (P1AA1199). P1AA1199 showed similar to the HeLa-human 4-1BB-NFκB-luc reporter cell line assay (see FIG. 2A, FIG. 2B, and FIG. 2C) for same measured parameters the tendency to display a lower EC₅₀ value (CD25 expression) or a lower plateau in the displayed curve (CD137 (4-1BB) expression) compared to construct 2.4. Shown are the mean+/−SD of three technical replicates of each measured point.

FIG. 4 demonstrates that the binding to CD19⁺ B cells of the CD19-targeted 4-1BB ligand trimer-containing antigen binding molecules of the invention is comparable to the binding of the control molecule CD19 (2B11)-targeted 4-1BB ligand trimer-containing antigen binding molecule construct 4.4 (CD19-4-1BBL Ab).

FIG. 5A shows the binding of different CD19-targeted 4-1BB ligand trimer-containing antigen binding molecules of the invention (P1AA1233, P1AA0776 and P1AA1258) to 4-1BB on activated CD4+ T cells. The data are comparable with those obtained for construct 4.4 (CD19-4-1BBL Ab).

FIG. 5B shows the binding of different CD19-targeted 4-1BB ligand trimer-containing antigen binding molecules of the invention (P1AA1233, P1AA0776 and P1AA1258) to 4-1BB on activated CD8+ T cells. The data are comparable with those obtained for construct 4.4 (CD19-4-1BBL Ab).

FIG. 5C shows the binding of different CD19-targeted 4-1BB ligand trimer-containing antigen binding molecules of the invention (P1AA1233, P1AA0776 and P1AA1258) to 4-1BB on NK cells. The data are comparable with those obtained for construct 4.4 (CD19-4-1BBL Ab).

FIG. 6 shows the biological activity of the different CD19-targeted 4-1BB ligand trimer-containing antigen binding molecules of the invention (P1AA1233, P1AA0776 and P1AA1258). The biological activity of the molecules is measured based on the release of effector function molecule IFNγ by 4-1BB-co-stimulated T cells and NK cells in PBMCs. Molecules of the invention are able to activate T cells and NK cells to produce a similar amount of IFNγ compared to construct 4.4 (CD19-4-1BBL Ab), whereas the untargeted control D was not able to induce IFNγ release.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

The term “antigen binding domain” refers to the part of an antigen binding molecule that specifically binds to an antigenic determinant. In one aspect, the antigen binding domain is able to activate signaling through its target cell antigen. In a particular aspect, the antigen binding domain is able to direct the entity to which it is attached (e.g. the TNF family ligand trimer) to a target site, for example to a specific type of tumor cell or tumor stroma bearing the antigenic determinant or on T cells. Antigen binding domains include the area or fragment of an antibody which specifically binds to and is complementary to part or all of an antigen. In addition, antigen binding domains include scaffold antigen binding proteins as further defined herein, e.g. binding domains which are based on designed repeat proteins or designed repeat domains (see e.g. WO 2002/020565). In particular, an antigen binding domain is comprised of a first part and a second part, wherein the first part comprises an antibody light chain variable region (VL) and the second part comprises an antibody heavy chain variable region (VH) or vice versa.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding domains in an antigen binding molecule. As such, the terms “bivalent”, “tetravalent”, and “hexavalent” denote the presence of two binding sites, four binding sites, and six binding sites, respectively, in an antigen binding molecule. Accordingly, “monovalent” means that there is only one antigen binding domain present in the molecule that is capable of specific binding to an antigen.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)2 fragment that has two antigen-combining sites (two Fab fragments) and a part of the Fc region.

Included into the term “Fab fragment” is also “cross-Fab fragment” or “xFab fragment” or “crossover Fab fragment”. This term refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the VH with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), VNAR fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin).

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 17, 455-462 (2004) and EP 1641818A1.

Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or VNAR fragments derived from sharks.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIACORE® instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “tumor associated antigen” as used herein refers to an antigenic determinant presented on the surface of a target cell, which is a cell in a tumor such as a cancer cell, a cell of the tumor stroma or a B cell. In certain embodiments, the tumor associated antigen is Fibroblast Activation Protein (FAP) or CD19.

The term “capable of specific binding to Fibroblast activation protein (FAP)” refers to an antigen binding molecule that is capable of binding FAP with sufficient affinity such that the antigen binding molecule is useful as a diagnostic and/or therapeutic agent in targeting FAP. The antigen binding molecule includes but is not limited to, antibodies, Fab molecules, crossover Fab molecules, single chain Fab molecules, Fv molecules, scFv molecules, single domain antibodies, and VH and scaffold antigen binding protein. In one aspect, the extent of binding of an anti-FAP antigen binding molecule to an unrelated, non-FAP protein is less than about 10% of the binding of the antigen binding molecule to FAP as measured, e.g., by Surface Plasmon Resonance (SPR).

In particular, an antigen binding molecule that is capable of specific binding to FAP has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³M, e.g., from 10⁻⁹M to 10⁻¹³ M). In certain embodiments, an anti-FAP antigen binding molecule binds to FAP from different species. In particular, the anti-FAP antigen binding molecule binds to human, cynomolgus and mouse FAP.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO: 57), or NCBI (www.ncbi.nlm.nih.gov/) RefSeq NP_004451.2. The extracellular domain (ECD) of human FAP extends from amino acid position 26 to 760. The amino acid sequence of mouse FAP is shown in UniProt accession no. P97321 (version 126, SEQ ID NO: 58), or NCBI RefSeq NP_032012.1. The extracellular domain (ECD) of mouse FAP extends from amino acid position 26 to 761. Preferably, an anti-FAP binding molecule of the invention binds to the extracellular domain of FAP. Exemplary anti-FAP binding molecules are described in International Patent Application No. WO 2012/020006 A2.

The term “capable of specific binding to CD19” refers to an antigen binding molecule that is capable of binding to CD19 with sufficient affinity such that the antigen binding molecule is useful as a diagnostic and/or therapeutic agent in targeting CD19. The antigen binding molecule includes but is not limited to, antibodies, Fab molecules, crossover Fab molecules, single chain Fab molecules, Fv molecules, scFv molecules, single domain antibodies, and VH and scaffold antigen binding protein. In one aspect, the extent of binding of an anti-CD19 antigen binding molecule to an unrelated, non-CD19 protein is less than about 10% of the binding of the antigen binding molecule to CD19 as measured, e.g., by Surface Plasmon Resonance (SPR). In particular, an antigen binding molecule that is capable of specific binding to CD19 has a dissociation constant (IQ) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³M, e.g., from 10⁻⁹M to 10⁻¹³ M). In certain embodiments, an anti-CD19 antigen binding molecule binds to human CD19.

The term “CD19” refers to B-lymphocyte antigen CD19, also known as B-lymphocyte surface antigen B4 or T-cell surface antigen Leu-12 and includes any native CD19 from any vertebrate source, including mammals such as primates (e.g. humans) non-human primates (e.g. cynomolgus monkeys) and rodents (e.g. mice and rats), unless otherwise indicated. The amino acid sequence of human CD19 is shown in Uniprot accession no. P15391 (version 160, SEQ ID NO: 59). The term encompasses “full-length” unprocessed human CD19 as well as any form of human CD19 that results from processing in the cell as long as the antibody as reported herein binds thereto. CD19 is a structurally distinct cell surface receptor expressed on the surface of human B cells, including, but not limited to, pre-B cells, B cells in early development {i.e., immature B cells), mature B cells through terminal differentiation into plasma cells, and malignant B cells. CD19 is expressed by most pre-B acute lymphoblastic leukemias (ALL), non-Hodgkin's lymphomas, B cell chronic lymphocytic leukemias (CLL), pro-lymphocytic leukemias, hairy cell leukemias, common acute lymphocytic leukemias, and some Null-acute lymphoblastic leukemias. The expression of CD19 on plasma cells further suggests it may be expressed on differentiated B cell tumors such as multiple myeloma. Therefore, the CD19 antigen is a target for immunotherapy in the treatment of non-Hodgkin's lymphoma, chronic lymphocytic leukemia and/or acute lymphoblastic leukemia. Exemplary anti-FAP binding molecule of the invention binds to the extracellular domain of FAP. Exemplary anti-CD19 antibodies are described in International Patent Application Nos. WO 2017/055328 or WO 2017/055541 A1.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen binding specificity.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR (e.g. CDR) residues and other residues in the variable domain (e.g., FR residues) are numbered herein according to Kabat et al., supra.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340 (EU numbering system according to Kabat). In one embodiment, a carbohydrate chain is attached to the CH2 domain. The CH2 domain herein may be a native sequence CH2 domain or variant CH2 domain. In one aspect, a CH2 domain comprises the amino acid sequence of SEQ ID NO: 60. The “CH3 domain” comprises the stretch of residues C-terminal to a CH2 domain in an Fc region (i.e. from an amino acid residue at about position 341 to an amino acid residue at about position 447 of an IgG). In one aspect, a CH3 domain comprises the amino acid sequence of SEQ ID NO: 61 (without the the C-terminal lysine). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “wild-type Fe domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 106 (IgG1, caucasian allotype), SEQ ID NO: 107 (IgG1, afroamerican allotype), SEQ ID NO: 108 (IgG2), SEQ ID NO: 109 (IgG3) and SEQ ID NO: 110 (IgG4).

The term “variant (human) Fe domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, Md., 1991.

A “region equivalent to the Fe region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “CH1 domain” denotes the part of an antibody heavy chain polypeptide that extends approximately from EU position 118 to EU position 215 (EU numbering system). In one embodiment a CH1 domain comprises the amino acid sequence of SEQ ID NO: 62. Usually, a segment having the amino acid sequence of EPKSC (SEQ ID NO: 99) is following to link the CH1 domain to the hinge region.

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083). In one aspect, the hinge region has the amino acid sequence DKTHTCPXCP (SEQ ID NO: 63), wherein X is either S or P. In one aspect, the hinge region has the amino acid sequence HTCPXCP (SEQ ID NO: 64), wherein X is either S or P. In one aspect, the hinge region has the amino acid sequence CPXCP (SEQ ID NO: 65), wherein X is either S or P.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “TNF ligand family member” or “TNF family ligand” refers to a proinflammatory cytokine. Cytokines in general, and in particular the members of the TNF ligand family, play a crucial role in the stimulation and coordination of the immune system. At present, nineteen cyctokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors.

Members of the TNF ligand family are selected from the group consisting of Lymphotoxin α (also known as LTA or TNFSF1), TNF (also known as TNFSF2), LTβ (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated. In specific embodiments of the invention, the TNF ligand family member is selected from the group consisting of OX40L, FasL, CD27L, TRAIL, 4-1BBL, CD40L and GITRL. In a particular embodiment, the TNF ligand family member is 4-1BBL.

Further information, in particular sequences, of the TNF ligand family members may be obtained from publically accessible databases such as Uniprot (www.uniprot.org). For instance, the human TNF ligands have the following amino acid sequences: human Lymphotoxin α (UniProt accession no. P01374, SEQ ID NO: 66), human TNF (UniProt accession no. P01375, SEQ ID NO: 67), human Lymphotoxin β (UniProt accession no. Q06643, SEQ ID NO: 68), human OX40L (UniProt accession no. P23510, SEQ ID NO: 69), human CD40L (UniProt accession no. P29965, SEQ ID NO: 70), human FasL (UniProt accession no. P48023, SEQ ID NO: 71), human CD27L (UniProt accession no. P32970, SEQ ID NO: 72), human CD30L (UniProt accession no. P32971, SEQ ID NO: 73, 4-1BBL (UniProt accession no. P41273, SEQ ID NO: 74), TRAIL (UniProt accession no. P50591, SEQ ID NO: 75), RANKL (UniProt accession no. 014788, SEQ ID NO: 76), TWEAK (UniProt accession no. 043508, SEQ ID NO: 77), APRIL (UniProt accession no. 075888, SEQ ID NO: 78), BAFF (UniProt accession no. Q9Y275, SEQ ID NO: 79), LIGHT (UniProt accession no. 043557, SEQ ID NO: 80), TL1A (UniProt accession no. 095150, SEQ ID NO: 81), GITRL (UniProt accession no. Q9UNG2, SEQ ID NO: 82) and ectodysplasin A (UniProt accession no. Q92838, SEQ ID NO: 83).

An “ectodomain” is the domain of a membrane protein that extends into the extracellular space (i.e. the space outside the target cell). Ectodomains are usually the parts of proteins that initiate contact with surfaces, which leads to signal transduction. The ectodomain of TNF ligand family member as defined herein thus refers to the part of the TNF ligand protein that extends into the extracellular space (the extracellular domain), but also includes shorter parts or fragments thereof that are responsible for the trimerization and for the binding to the corresponding TNF receptor. The term “ectodomain of a TNF ligand family member or a fragment thereof” thus refers to the extracellular domain of the TNF ligand family member that forms the extracellular domain or to parts thereof that are still able to bind to the receptor (receptor binding domain).

The term “costimulatory TNF ligand family member” or “costimulatory TNF family ligand” refers to a subgroup of TNF ligand family members, which are able to costimulate proliferation and cytokine production of T-cells. These TNF family ligands can costimulate TCR signals upon interaction with their corresponding TNF receptors and the interaction with their receptors leads to recruitment of TNFR-associated factors (TRAF), which initiate signalling cascades that result in T-cell activation. Costimulatory TNF family ligands are selected from the group consisting of 4-1BBL, OX40L, GITRL, CD70, CD30L and LIGHT, more particularly the costimulatory TNF ligand family member is selected from 4-1BBL and OX40L.

As described herein before, 4-1BBL is a type II transmembrane protein and one member of the TNF ligand family. Complete or full length 4-1BBL having the amino acid sequence of SEQ ID NO: 74 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO: 84) form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO: 4 (amino acids 52-254 of human 4-1BBL), SEQ ID NO: 1 (amino acids 71-254 of human 4-1BBL), SEQ ID NO: 3 (amino acids 80-254 of human 4-1BBL) and SEQ ID NO: 2 (amino acids 85-254 of human 4-1BBL) or a polypeptide having an amino acid sequence selected from SEQ ID NO: 5 (amino acids 71-248 of human 4-1BBL), SEQ ID NO: 8 (amino acids 52-248 of human 4-1BBL), SEQ ID NO: 7 (amino acids 80-248 of human 4-1BBL) and SEQ ID NO: 6 (amino acids 85-248 of human 4-1BBL), but also other fragments of the ectodomain capable of trimerization are included herein.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 1 and 4, in particular 2, i.e. the peptides selected from the group consisting of GGGGS (SEQ ID NO: 41), GGGGSGGGGS (SEQ ID NO: 42), SGGGGSGGGG (SEQ ID NO: 43), GGGGGSGGGGSSGGGGS (SEQ ID NO: 44), (G₄S)₃ or GGGGSGGGGSGGGGS (SEQ ID NO: 45), GGGGSGGGGSGGGG or G4(SG4)₂ (SEQ ID NO: 46), and (G₄S)₄ or GGGGSGGGGSGGGGSGGGGS (SEQ ID NO: 47), but also include the sequences GSPGSSSSGS (SEQ ID NO: 48), GSGSGSGS (SEQ ID NO:49), GSGSGNGS (SEQ ID NO: 50), GGSGSGSG (SEQ ID NO: 51), GGSGSG (SEQ ID NO: 52), GGSG (SEQ ID NO: 53), GGSGNGSG (SEQ ID NO: 54), GGNGSGSG (SEQ ID NO: 55) and GGNGSG (SEQ ID NO: 56). Peptide linkers of particular interest are (G4S)₁ or GGGGS (SEQ ID NO: 41), (G₄S)₂ or GGGGSGGGGS (SEQ ID NO: 42) and GGGGGSGGGGSSGGGGS (SEQ ID NO: 44), more particularly (G₄S)₂ or GGGGSGGGGS (SEQ ID NO: 42).

A “spacer domain” according to the present invention is a polypeptide forming a structural domain after folding. Thus, the spacer domain can be smaller than 100 amino acid residues, but needs to be structurally confined to fix the binding motifs. Exemplary spacer domains are pentameric coil-coils, antibody hinge regions or antibody Fc regions or fragments thereof. The spacer domain is is a dimerization domain, i.e. the the spacer domain comprises amino acids that are able to provide the dimerization functionality.

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “fusion polypeptide” or “single fusion polypeptide” as used herein refers to a single chain polypeptide composed of different components such as the ectodomain of a TNF ligand family member that are fused to each either directly or via a peptide linker. By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the TNF ligand trimer-containing antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include TNF family ligand trimer-containing antigen binding molecule with an N-terminal methionyl residue. Other insertional variants of the molecule include the fusion to the N- or C-terminus to a polypeptide which increases the serum half-life of the TNF ligand trimer-containing antigen binding molecules.

In certain aspects, the TNF family ligand trimer-containing antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of TNF family ligand trimer-containing antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the TNF family ligand trimer-containing antigen binding molecules of the invention include those with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the TNF family ligand trimer-containing antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antigen binding molecules may be generated as described, e.g., in U.S. Pat. No. 7,521,541.

In certain aspects, the TNF family ligand trimer-containing antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

In another aspect, immunoconjugates of the TNF family ligand trimer-containing antigen binding molecules provided herein maybe obtained. An “immunoconjugate” is an antibody conjugated to one or more heterologous molecule(s), including but not limited to a cytotoxic agent.

The term “nucleic acid” refers to an isolated nucleic acid molecule or construct, e.g. messenger RNA (mRNA), virally-derived RNA, or plasmid DNA (pDNA). Nucleic acid may comprise a conventional phosphodiester bond or a non-conventional bond (e.g. an amide bond, such as found in peptide nucleic acids (PNA). The term “nucleic acid” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant nucleic acid encoding a polypeptide contained in a vector is considered isolated for the purposes of the present invention. Further examples of isolated nucleic acid include recombinant polynucleotides maintained in heterologous host cells or purified (partially or substantially) polynucleotides in solution. An isolated nucleic acid includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, carcinoma, lymphoma, blastoma, sarcoma, leukemia, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colorectal cancer (CRC), pancreatic cancer, breast cancer, triple-negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, melanoma, multiple myeloma, B-cell cancer (lymphoma), chronic lymphocytic leukemia (CLL), acute lymphoblastic leukemia (ALL), hairy cell leukemia, chronic myeloblastic leukemia, including refractory versions of any of the above cancers, or a combination of one or more of the above cancers.

TNF Family Ligand Trimer-Containing Antigen Binding Molecules of the Invention

The invention provides novel TNF family ligand trimer-containing antigen binding molecules with particularly advantageous properties such as producibility, stability, binding affinity, biological activity, targeting efficiency and reduced toxicity.

In particular, the present invention describes how a trimeric TNF ligand can be efficiently fused to an antibody so that the trimeric ligand is correctly assembled and fully functional. For a molecule intended to be developed towards clinical application, aggregates of functionally active molecules have to be avoided, meaning the purity and stability of natural ligand fusion is very critical. Importantly, in the antigen binding molecules of the invention, all three TNF ligands are fused to the heavy chains of the antibody. Thus, the problem of correct pairing between heavy and light chains can be avoided.

Focusing on an antibody-based architecture is guided by the good pharmacokinetic properties of the antibodies in general. The antibody architecture is stabile compared to other protein; their expression is also very robust using different cell lines. Their Fc part interacts with the FcRn receptor and therefore preserves the molecules from rapid elimination through intracellular degradation. Important is also that the constructs are expressable with reasonably good titers and produce a good ratio of the wished product. The antibody architecture is stabile compared to other protein; their expression is also very robust using different cell lines.

Here, we demonstrate the advantage of using the antibody architecture combined with the contorsbody principle. The latest consists of fusing one part of an antigen binding domain, for example the heavy chain part of a Fab molecule, on the N-terminus of a dimerizing or a multimerizing spacer domain (in this case, a monomeric Fc moiety), and the other part, e.g. a light chain part of the Fab molecule, to the C-terminus of the same dimerizing or multimerizing spacer domain.

In the case of trimeric TNF ligands, a dimer of the ligand can be fused to the C-terminal part of an Fc whereas the third ligand can be fused to the N-terminal part of the Fc to form a first half of the full antigen binding molecule. Another standard set of chain pairing forms a “standard” Fab-Fc combination as the second half of the antigen binding molecule. Or the second half of the molecule is comprised of a single circular fusion polypeptide wherein the heavy chain of the Fab is fused to the N-terminus of the Fc and the light chain of the Fab is fused to the C-terminus of the Fc (“contorsbody”). As the Fc “knob-into-hole” technology is used to differentiate the two heavy chains, a hetero-dimerization occurs between the two different Fc parts to form the final molecule. Having all 3 ligands on a single chain helps to get association of the monomers from the same chain; as soon as the polypeptide is build and the folding of each subdomain is achieved, the different part assembles preferably with a partner from the same polypeptide because of the relative high concentration of the latest compared to a domain from another polypeptide. If an incomplete trimerization of a molecule is able to form a complex with another monovalent or divalent form of a fused TNF ligand, this leads to side products that are either high molecule weight species or non-trimeric forms of the ligand. In another alternative, one TNF ligand can be fused to the N-terminal part of an Fc whereas the second ligand can be fused to the C-terminal part of the same Fc and the third ligand can be fused to the C-terminal part of the second Fc. Also in this case, all three ligands preferably assemble with each other as they are all fused to the Fc parts which are closely linked to each other by disulfide bonds.

As the expression rate of different polypeptidic chains is a key element for triggering further association, the present invention suggests avoiding the fusion of a TNF ligand to a short polypeptide chain (i.e. a light chain) because this may express much faster than longer polypeptide chains. In such case, the short polypeptide chains can associate with themselves and produce the main side-product, a trimeric TNF ligand lacking the Fc part and antigen binding domain of the final molecule. Depleting the short chain does not hinder the association of the heavy chains through their Fc part. Aggregation of incomplete molecules can thus be avoided if TNF ligands that have a tendency to trimerize by themselves are not fused to short chains or the short chains (light chains) are even missing.

Another focus of this invention is to keep both the first fusion polypeptide and the second fusion polypeptide of similar length. This is particularly fulfilled if one TNF ligand is fused to the C-terminus of a “regular” antibody heavy chain whereas the two other TNF ligands are fused on both sides of the complementary Fc part. Both Fc-containing chains have a molecular weight around 65 kD and are then supposed to be processed and folded similarly fast to get a 1:1 stoechiometry in the medium. The expected association between the knob-containing and the hole-containing Fc part is then driving the association of the trimeric ligand provided by two polypeptidic chains.

The TNF family ligand trimer-containing antigen binding molecules are thus particularly characterized by their producibility (low tendency to build aggregation products during the preparation) and their stability. The molecules are also called “TNF ligand contorsbodies”.

In a first aspect, the invention provides a TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain of said TNF ligand family member or a fragment thereof is fused either directly or via a second peptide linker to the C-terminus of the spacer domain,
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is fused either directly or via a third peptide linker to the C-terminus of the spacer domain or is present in form of a light chain, and
(c) a third ectodomain of said TNF ligand family member or a fragment thereof that is fused either directly or via a fourth peptide linker to
   either the C-terminus of the second ectodomain of said TNF ligand family member in the first fusion polypeptide or to the C-terminus of the spacer domain in the second fusion polypeptide, or
   in case the second part of the antigen binding domain is fused to the C-terminus of the spacer domain of the second fusion protein, to the C-terminus of the second ectodomain of said TNF ligand family member in the first fusion polypeptide,
wherein the spacer domain of the first fusion polypeptide and the spacer domain of the second fusion polypeptide are associated covalently to each other by a disulfide bond.

In one aspect, the invention provides a TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain and third ectodomain of said TNF ligand family member or a fragment thereof are fused to each other and either directly or via a second peptide linker to the C-terminus of the spacer domain, and
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is present in form of a light chain, and
wherein the spacer domain of the first fusion polypeptide and the spacer domain of the second fusion polypeptide are associated covalently to each other by a disulfide bond.

In a second aspect, the invention provides a TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain and third ectodomain of said TNF ligand family member or a fragment thereof are fused to each other and either directly or via a second peptide linker to the C-terminus of the spacer domain, and
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is fused either directly or via a third peptide linker to the C-terminus of the spacer domain, and
wherein the spacer domain of the first fusion polypeptide and the spacer domain of the second fusion polypeptide are associated covalently to each other by a disulfide bond.

In a third aspect, the invention provides a TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain of said TNF ligand family member or a fragment thereof is fused either directly or via a second peptide linker to the C-terminus of the spacer domain,
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is present in form of a light chain and wherein a third ectodomain of said TNF ligand family member or a fragment thereof is fused either directly or via a fourth peptide linker to the C-terminus of the spacer domain, and
wherein the spacer domain of the first fusion polypeptide and the spacer domain of the second fusion polypeptide are associated covalently to each other by a disulfide bond.

In a particular aspect, the invention provides a TNF family ligand trimer-containing antigen binding molecule as defined herein before, wherein the first part of the antigen binding domain comprises an antibody heavy chain variable domain and the second part of the antigen binding domain comprises an antibody light chain variable domain or vice versa. In one specific aspect, the antibody heavy chain variable domain is fused to the N-terminus of the spacer domain and the antibody light chain variable domain is fused to the C-terminus of the same spacer domain. In another aspect, the antibody heavy chain variable domain is fused to the C-terminus of the spacer domain and the antibody light chain variable domain is present on a different chain, in particular a light chain. In a further aspect, the invention provides a TNF family ligand trimer-containing antigen binding molecule as defined herein before, wherein the first part of the antigen binding domain is an antibody heavy chain Fab fragment and the second part of the antigen binding domain is an antibody light chain Fab fragment or vice versa. In one specific aspect, the antibody heavy chain Fab fragment is fused to the N-terminus of the spacer domain and the antibody light chain Fab fragment is fused to the C-terminus of the same spacer domain. In one aspect, the first part of the antigen binding domain and the second part of the antigen binding domain are associated covalently to each other by a disulfide bond.

As described above, the TNF family ligand trimer-containing antigen binding molecule comprises a first and a second fusion polypeptide, both comprising a spacer domain, wherein the spacer domain of the first fusion polypeptide and the spacer domain of the second fusion polypeptide are associated covalently to each other by a disulfide bond.

In one aspect of the invention, the spacer domain comprises an antibody hinge region or a (C-terminal) fragment thereof and an antibody CH2 domain or a (N-terminal) fragment thereof. In another aspect, the spacer domain comprises an antibody hinge region or a fragment thereof, an antibody CH2 domain, and an antibody CH3 domain or a fragment thereof. In a further aspect, the invention comprises a TNF family ligand trimer-containing antigen binding molecule, wherein the spacer domain comprises an antibody hinge region or a fragment thereof and a human Fc region (domain). In particular, the human Fc domain is a human IgG1, IgG2, IgG3 or IgG4 Fc domain, more particularly, the spacer domain of the TNF family ligand trimer-containing antigen binding molecule comprises a human IgG1 domain.

In one aspect, the invention relates to TNF family ligand trimer-containing antigen binding molecule as described herein before, wherein the first, second, third and fourth peptide linker is present and consists of an amino acid sequence selected from the group consisting of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55 and SEQ ID NO: 56. In particular, the peptide linker consists of an amino acid sequence selected from SEQ ID NO: 42 and SEQ ID NO: 44. More particularly, the peptide linker consists of an amino acid sequence of SEQ ID NO: 42.

In a further aspect of the invention, the spacer domain of the first fusion polypeptide and the spacer domain of the second fusion polypeptide comprise modifications promoting the association of the first and second fusion polypeptide. In a particular aspect, the spacer domain of the first fusion polypeptide comprises holes and the spacer domain of the second fusion polypeptide comprises knobs according to the knobs into hole method.

Fc Domain Modifications Promoting Heterodimerization

In one aspect, the TNF family ligand trimer-containing antigen binding molecules of the invention comprise (a) a first fusion polypeptide as defined herein before and a second fusion polypeptide as defined herein before, wherein the first and second fusion polypeptide comprise modifications promoting the association of the first and second fusion polypeptide. Typically, these modifications are introduced in the Fc domains. Recombinant co-expression of the two structurally different fusion polypeptides and subsequent dimerization would lead to several possible combinations of the two polypeptides. In order to improve the yield and purity of the TNF family ligand trimer-containing antigen binding molecules in recombinant production, it will thus be advantageous to introduce in the Fc domain of the TNF family ligand trimer-containing antigen binding molecules of the invention modifications promoting the association of the desired polypeptides.

The site of most extensive protein-protein interaction between the two subunits of a human IgG Fc domain is in the CH3 domain of the Fc domain. Thus, said modification is particularly in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, in a particular aspect, the invention relates to a TNF family ligand trimer-containing antigen binding molecule as described herein before which comprises an IgG molecule, wherein the Fc part of the first heavy chain comprises a first dimerization module and the Fc part of the second heavy chain comprises a second dimerization module allowing a heterodimerization of the two heavy chains of the IgG molecule and the first dimerization module comprises knobs and the second dimerization module comprises holes according to the knob into hole technology.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

The CH3 domains in the first and second fusion polypeptide as reported herein can be altered by the “knob-into-holes” technology which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

Accordingly, in a particular aspect, in the CH3 domain of the first subunit of the Fc domain of the TNF family ligand trimer-containing antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

In a specific aspect, in the CH3 domain of the first subunit of the Fc domain (“knobs chain”) the threonine residue at position 366 is replaced with a tryptophan residue (T366W), and in the CH3 domain of the second subunit of the Fc domain the tyrosine residue at position 407 is replaced with a valine residue (Y407V). More particularly, in the second subunit of the Fc domain (“hole chain”) additionally the threonine residue at position 366 is replaced with a serine residue (T366S) and the leucine residue at position 368 is replaced with an alanine residue (L368A). More particularly, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). The introduction of these two cysteine residues results in the formation of a disulfide bridge between the two subunits of the Fc domain. The disulfide bridge further stabilizes the dimer (Carter, J Immunol Methods 248, 7-15 (2001)).

But also other knobs-in-holes technologies as described by EP 1870459 A1, can be used alternatively or additionally. In one embodiment the multicircular fusion polypeptide as reported herein comprises the R409D and K370E mutations in the CH3 domain of the “knobs chain” and the D399K and E357K mutations in the CH3 domain of the “hole-chain” (numbering according to Kabat EU index).

In a further aspect, the TNF family ligand trimer-containing antigen binding molecule may comprises the Y349C and T366W mutations in one of the two CH3 domains and the S354C, T366S, L368A and Y407V mutations in the other of the two CH3 domains, or the TNF family ligand trimer-containing antigen binding molecule as reported herein comprises the Y349C and T366W mutations in one of the two CH3 domains and the S354C, T366S, L368A and Y407V mutations in the other of the two CH3 domains and additionally the R409D and K370E mutations in the CH3 domain of the “knobs chain” and the D399K and E357K mutations in the CH3 domain of the “hole chain” (numbering according to the Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Apart from the “knob-into-hole technology” other techniques for modifying the CH3 domains of the heavy chains to enforce heterodimerization are known in the art. These technologies, especially the ones described in WO 96/27011, WO 98/050431, EP 1870459, WO 2007/110205, WO 2007/147901, WO 2010/129304, WO 2011/90754, WO 2011/143545, WO 2012/058768, WO 2013/157954 and WO 2013/096291 are contemplated herein as alternatives to the “knob-into-hole technology” in combination with a TNF family ligand trimer-containing antigen binding molecule as described herein.

In one aspect, charged amino acids with opposite charges at specific amino acid positions in the CH3/CH3-domain-interface between both, the first and the second heavy chain are introduced to further promote the association of the desired polypeptides. Accordingly, this aspect relates to antigen binding molecules as disclosed herein, wherein in the tertiary structure of the antibody the CH3 domain of the first heavy chain and the CH3 domain of the second heavy chain an interface is formed that is located between the respective antibody CH3 domains, wherein the respective amino acid sequences of the CH3 domain of the first heavy chain and the CH3 domain of the second heavy chain each comprise a set of amino acids that is located within said interface in the tertiary structure of the circular fusion polypeptide, and wherein from the set of amino acids that is located in the interface in the CH3 domain of one heavy chain a first amino acid is substituted by a positively charged amino acid and from the set of amino acids that is located in the interface in the CH3 domain of the other heavy chain a second amino acid is substituted by a negatively charged amino acid. The TNF family ligand trimer-containing antigen binding molecule according to this aspect is herein also referred to as “CH3(+/−)-engineered TNF family ligand trimer-containing antigen binding molecule” (wherein the abbreviation “+/−” stands for the oppositely charged amino acids that were introduced in the respective CH3 domains). In one aspect of said CH3(+/−)-engineered TNF family ligand trimer-containing antigen binding molecule as reported herein the positively charged amino acid is selected from K, R and H, and the negatively charged amino acid is selected from E or D. In another aspect, in said CH3(+/−)-engineered TNF family ligand trimer-containing antigen binding molecule as reported herein the positively charged amino acid is selected from K and R, and the negatively charged amino acid is selected from E or D. In a further aspect, in said CH3(+/−)-engineered TNF family ligand trimer-containing antigen binding molecule as reported herein the positively charged amino acid is K, and the negatively charged amino acid is E. In one aspect, in said CH3(+/−)-engineered TNF family ligand trimer-containing antigen binding molecule as reported herein in the CH3 domain of one heavy chain the amino acid R at position 409 is substituted by D and the amino acid K at position is substituted by E, and in the CH3 domain of the other heavy chain the amino acid D at position 399 is substituted by K and the amino acid E at position 357 is substituted by K (numbering according to Kabat EU index).

In a further aspect of the invention, the IgG1 Fc domain comprises one or more amino acid substitution that reduces binding to an Fc receptor, in particular towards Fcγ receptor.

Fc Domain Modifications Reducing Fc Receptor Binding and/or Effector Function

The TNF family ligand trimer-containing antigen binding molecules of the invention may comprise as a spacer domain the heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. The Fc domain confers favorable pharmacokinetic properties to the antigen binding molecules of the invention, including a long serum half-life which contributes to good accumulation in the target tissue and a favorable tissue-blood distribution ratio.

In certain aspects, provided is a TNF family ligand trimer-containing antigen binding molecule that possesses some but not all effector functions, which make it a desirable candidate for applications in which the half-life in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the circular fusion polypeptide lacks FcγR binding (hence likely lacking ADCC activity), but retains FcRn binding ability.

Accordingly, in particular aspects, the Fc domain of the TNF family ligand trimer-containing antigen binding molecule of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of a TNF family ligand trimer-containing antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides a TNF family ligand trimer-containing antigen binding molecule, wherein the spacer domain comprises Fc domain that comprises one or more amino acid substitution that reduces binding to an Fc receptor, in particular towards Fcγ receptor.

In one aspect, the Fc domain of the TNF family ligand trimer-containing antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is a trimeric TNF family ligand-containing antigen binding molecule according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions. “EU numbering” refers to the numbering according to EU index of Kabat et al, Sequences of Proteins of Immunological Interest, 5th Ed. Public Health Service, National Institutes of Health, Bethesda, Md., 1991.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position 5228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIACORE® instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

In particular aspects, the TNF family ligand trimer-containing antigen binding molecule comprises all positions according to EU index of Kabat)

i) a homodimeric Fc-region of the human IgG1 subclass optionally with the mutations P329G, L234A and L235A, or

ii) a homodimeric Fc-region of the human IgG4 subclass optionally with the mutations P329G, S228P and L235E, or

iii) a homodimeric Fc-region of the human IgG1 subclass optionally with the mutations P329G, L234A, L235A, I253A, H310A, and H435A, or optionally with the mutations P329G, L234A, L235A, H310A, H433A, and Y436A, or

iv) a heterodimeric Fc-region whereof

a) one Fc-region polypeptide comprises the mutation T366W, and the other Fc-region polypeptide comprises the mutations T366S, L368A and Y407V, or

b) one Fc-region polypeptide comprises the mutations T366W and Y349C, and the other Fc-region polypeptide comprises the mutations T366S, L368A, Y407V, and S354C, or

c) one Fc-region polypeptide comprises the mutations T366W and S354C, and the other Fc-region polypeptide comprises the mutations T366S, L368A, Y407V and Y349C,

or

v) a heterodimeric Fc-region of the human IgG1 subclass whereof both Fc-region polypeptides comprise the mutations P329G, L234A and L235A and

a) one Fc-region polypeptide comprises the mutation T366W, and the other Fc-region polypeptide comprises the mutations T366S, L368A and Y407V, or

b) one Fc-region polypeptide comprises the mutations T366W and Y349C, and the other Fc-region polypeptide comprises the mutations T366S, L368A, Y407V, and S354C, or

c) one Fc-region polypeptide comprises the mutations T366W and S354C, and the other Fc-region polypeptide comprises the mutations T366S, L368A, Y407V and Y349C,

or

vi) a heterodimeric Fc-region of the human IgG4 subclass whereof both Fc-region polypeptides comprise the mutations P329G, S228P and L235E and

a) one Fc-region polypeptide comprises the mutation T366W, and the other Fc-region polypeptide comprises the mutations T366S, L368A and Y407V, or

b) one Fc-region polypeptide comprises the mutations T366W and Y349C, and the other Fc-region polypeptide comprises the mutations T366S, L368A, Y407V, and S354C, or

c) one Fc-region polypeptide comprises the mutations T366W and S354C, and the other Fc-region polypeptide comprises the mutations T366S, L368A, Y407V and Y349C, or

vii) a combination of one of i), ii), and iii) with one of vi), v) and vi).

The C-terminus of the Fc domains comprised in the TNF family ligand trimer-containing antigen binding molecules as reported herein can be a complete C-terminus ending with the amino acid residues PGK. The C-terminus can be a shortened C-terminus in which one or two of the C-terminal amino acid residues have been removed. In one preferred embodiment the C-terminus is a shortened C-terminus ending with the amino acid residues PG.

Particular TNF Family Ligand Trimer-Containing Antigen Binding Molecules

In another aspect, the invention provides a TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain of said TNF ligand family member or a fragment thereof is fused either directly or via a second peptide linker to the C-terminus of the spacer domain,
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is fused either directly or via a third peptide linker to the C-terminus of the spacer domain or is present in form of a light chain, and
(c) a third ectodomain of said TNF ligand family member or a fragment thereof that is fused either directly or via a fourth peptide linker to
   either the C-terminus of the second ectodomain of said TNF ligand family member in the first fusion polypeptide or to the C-terminus of the spacer domain in the second fusion polypeptide, or
   in case the second part of the Fab molecule consisting of a variable antigen binding domain and a constant domain is fused to the C-terminus of the spacer domain of the second fusion protein, to the C-terminus of the second ectodomain of said TNF ligand family member in the first fusion polypeptide,
wherein the spacer domain of the first fusion polypeptide and the spacer domain of the second fusion polypeptide are associated covalently to each other by a disulfide bond and wherein the TNF ligand family member is one that costimulates human T-cell activation.

Thus, the invention relates to a TNF family ligand trimer-containing antigen binding molecule that costimulates human T-cell activation. In a particular aspect of the invention, the TNF family ligand is 4-1BBL. Antigen binding molecules of the invention comprising a 4-1BBL trimer are herein called 4-1BBL contorsbodies.

In a further aspect, provided is a TNF family ligand trimer-containing antigen binding molecule, wherein the ectodomain of the TNF ligand family member thus comprises the amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8, particularly the amino acid sequence of SEQ ID NO: 1 or SEQ ID NO: 5. More particularly, the ectodomain of the TNF ligand family member comprises the amino acid sequence of SEQ ID NO: 5.

In yet another aspect, provided is a TNF family ligand trimer-containing antigen binding molecule, wherein the molecule comprises three ectodomains of the TNF ligand family member, and in particular, all three ectodomains of of the TNF ligand family member comprise the same amino acid sequence.

The TNF family ligand trimer-containing antigen binding molecule of the invention further comprises an antigen binding domain consisting of a first and second part. In one aspect, the antigen binding domain is capable of specific binding to a tumor associated antigen. In a further aspect, the antigen binding domain is capable of specific binding to Fibroblast Activation Protein (FAP) or CD19.

In one aspect, the antigen binding domain is capable of specific binding to FAP. Molecules, wherein the antigen binding domain is capable of specific binding to FAP and wherein the TNF family ligand is 4-1BBL, are herein called FAP-4-1BBL contorsbodies.

Particularly, the antigen binding domain capable of specific binding to FAP comprises

(a) a heavy chain variable region (V_{H}FAP) comprising (i) CDR-H1 comprising the amino acid sequence of SEQ ID NO: 9, (ii) CDR-H2 comprising the amino acid sequence of SEQ ID NO:10, and (iii) CDR-H3 comprising the amino acid sequence of SEQ ID NO: 11, and a light chain variable region (V_{L}FAP) comprising (iv) CDR-L1 comprising the amino acid sequence of SEQ ID NO: 12, (v) CDR-L2 comprising the amino acid sequence of SEQ ID NO: 13, and (vi) CDR-L3 comprising the amino acid sequence of SEQ ID NO: 14, or
(b) a heavy chain variable region (V_{H}FAP) comprising (i) CDR-H1 comprising the amino acid sequence of SEQ ID NO: 15, (ii) CDR-H2 comprising the amino acid sequence of SEQ ID NO: 16, and (iii) CDR-H3 comprising the amino acid sequence of SEQ ID NO: 17, and a a light chain variable region (V_{L}FAP) comprising (iv) CDR-L1 comprising the amino acid sequence of SEQ ID NO: 18, (v) CDR-L2 comprising the amino acid sequence of SEQ ID NO: 19, and (vi) CDR-L3 comprising the amino acid sequence of SEQ ID NO: 20.

In a particular aspect, the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO: 21, and a light chain variable region (V_{L}FAP) comprising an amino acid sequence that is at least about 95%, 96%, 97%, 98%, 99% or 100% identical to the amino acid sequence of SEQ ID NO: 22, or (b) a heavy chain variable region (V_{H}FAP) comprising an amino acid sequence that is at least about 95%, 96%, 97%, 98%, 99% or 100% identical to the amino acid sequence of SEQ ID NO: 23, and a light chain variable region (V_{L}FAP) comprising an amino acid sequence that is at least about 95%, 96%, 97%, 98%, 99% or 100% identical to the amino acid sequence of SEQ ID NO: 24. Particularly, the antigen binding domain capable of specific binding to FAP comprises (a) a heavy chain variable region (V_{H}FAP) comprising the amino acid sequence of SEQ ID NO: 21 and a light chain variable region (V_{L}FAP) comprising an amino acid sequence of SEQ ID NO: 22, or (b) a heavy chain variable region (V_{H}FAP) comprising the amino acid sequence of SEQ ID NO: 23 and a light chain variable region (V_{L}FAP) comprising the amino acid sequence of SEQ ID NO: 24. More particularly, the antigen binding domain capable of specific binding to FAP comprises a heavy chain variable region (V_{H}FAP) comprising the amino acid sequence of SEQ ID NO: 21 and a light chain variable region (V_{L}FAP) comprising an amino acid sequence of SEQ ID NO: 22.

In one aspect, the TNF family ligand trimer-containing antigen binding molecule of the invention comprises

(a) a first fusion polypeptide comprising the amino acid sequence of SEQ ID NO: 85,
(b) a second fusion polypeptide comprising the amino acid sequence of SEQ ID NO: 86, and a light chain comprising the amino acid sequence of SEQ ID NO: 87.

In another aspect, the TNF family ligand trimer-containing antigen binding molecule of the invention comprises

(a) a first fusion polypeptide comprising the amino acid sequence of SEQ ID NO: 88,
(b) a second fusion polypeptide comprising the amino acid sequence of SEQ ID NO: 89, and a light chain comprising the amino acid sequence of SEQ ID NO: 87.

In another aspect, the TNF family ligand trimer-containing antigen binding molecule of the invention comprises

(a) a first fusion polypeptide comprising the amino acid sequence of SEQ ID NO: 90,
(b) a second fusion polypeptide comprising the amino acid sequence of SEQ ID NO: 89, and a light chain comprising the amino acid sequence of SEQ ID NO: 87.

In a further aspect, the TNF family ligand trimer-containing antigen binding molecule of the invention comprises

(a) a first fusion polypeptide comprising the amino acid sequence of SEQ ID NO: 85, and
(b) a second fusion polypeptide comprising the amino acid sequence of SEQ ID NO: 91, or
(a) a first fusion polypeptide comprising the amino acid sequence of SEQ ID NO: 85, and
(b) a second fusion polypeptide comprising the amino acid sequence of SEQ ID NO: 92.

In another aspect, the antigen binding domain is capable of specific binding to CD19. Molecules, wherein the antigen binding domain is capable of specific binding to CD19 and wherein the TNF family ligand is 4-1BBL, are herein called CD19-4-1BBL contorsbodies.

In particular, the antigen binding domain capable of specific binding to CD19 comprises

(a) a heavy chain variable region (V_{H}CD19) comprising (i) CDR-H1 comprising the amino acid sequence of SEQ ID NO: 25, (ii) CDR-H2 comprising the amino acid sequence of SEQ ID NO: 26, and (iii) CDR-H3 comprising the amino acid sequence of SEQ ID NO: 27, and a light chain variable region (V_{L}CD19) comprising (iv) CDR-L1 comprising the amino acid sequence of SEQ ID NO: 28, (v) CDR-L2 comprising the amino acid sequence of SEQ ID NO: 29, and (vi) CDR-L3 comprising the amino acid sequence of SEQ ID NO: 30, or
(b) a heavy chain variable region (V_{H}CD19) comprising (i) CDR-H1 comprising the amino acid sequence of SEQ ID NO: 31, (ii) CDR-H2 comprising the amino acid sequence of SEQ ID NO: 32, and (iii) CDR-H3 comprising the amino acid sequence of SEQ ID NO: 33, and a a light chain variable region (V_{L}CD19) comprising (iv) CDR-L1 comprising the amino acid sequence of SEQ ID NO: 34, (v) CDR-L2 comprising the amino acid sequence of SEQ ID NO: 35, and (vi) CDR-L3 comprising the amino acid sequence of SEQ ID NO: 36.

Particularly, the antigen binding domain capable of specific binding to FAP comprises (a) a heavy chain variable region (V_{H}CD19) comprising an amino acid sequence that is at least about 95%, 96%, 97%, 98%, 99% or 100% identical to the amino acid sequence of SEQ ID NO: 37, and a light chain variable region (V_{L}CD19) comprising an amino acid sequence that is at least about 95%, 96%, 97%, 98%, 99% or 100% identical to the amino acid sequence of SEQ ID NO: 38, or (b) a heavy chain variable region (V_{H}CD19) comprising an amino acid sequence that is at least about 95%, 96%, 97%, 98%, 99% or 100% identical to the amino acid sequence of SEQ ID NO: 39, and a light chain variable region (V_{L}CD19) comprising an amino acid sequence that is at least about 95%, 96%, 97%, 98%, 99% or 100% identical to the amino acid sequence of SEQ ID NO: 40. More particularly, the antigen binding domain capable of specific binding to FAP comprises (a) a heavy chain variable region (V_{H}CD19) comprising the amino acid sequence of SEQ ID NO: 37 and a light chain variable region (V_{L}CD19) comprising the amino acid sequence of SEQ ID NO: 38, or (b) a heavy chain variable region (V_{H}CD19) comprising the amino acid sequence of SEQ ID NO: 39 and a light chain variable region (V_{L}CD19) comprising the amino acid sequence of SEQ ID NO: 40. More particularly, the antigen binding domain capable of specific binding to FAP comprises (a) a heavy chain variable region (V_{H}CD19) comprising the amino acid sequence of SEQ ID NO: 37 and a light chain variable region (V_{L}CD19) comprising the amino acid sequence of SEQ ID NO: 38.

In one aspect, the TNF family ligand trimer-containing antigen binding molecule of the invention comprises

(a) a first fusion polypeptide comprising the amino acid sequence of SEQ ID NO: 88,
(b) a second fusion polypeptide comprising the amino acid sequence of SEQ ID NO: 99, and a light chain comprising the amino acid sequence of SEQ ID NO: 100.

In another aspect, the TNF family ligand trimer-containing antigen binding molecule of the invention comprises

(a) a first fusion polypeptide comprising the amino acid sequence of SEQ ID NO: 90,
(b) a second fusion polypeptide comprising the amino acid sequence of SEQ ID NO: 101, and a light chain comprising the amino acid sequence of SEQ ID NO: 100.

In another aspect, the TNF family ligand trimer-containing antigen binding molecule of the invention comprises

(a) a first fusion polypeptide comprising the amino acid sequence of SEQ ID NO: 85,
(b) a second fusion polypeptide comprising the amino acid sequence of SEQ ID NO: 102, and a light chain comprising the amino acid sequence of SEQ ID NO: 100.

Polynucleotides

The invention further provides isolated nucleic acid encoding a TNF family ligand trimer-containing antigen binding molecule as described herein or a fragment thereof.

The isolated nucleic acid encoding TNF ligand trimer-containing antigen binding molecules of the invention may be expressed as a single polynucleotide that encodes the entire antigen binding molecule or as multiple (e.g., two or more) polynucleotides that are co-expressed. Polypeptides encoded by nucleic acid that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated nucleic encodes the entire TNF family ligand trimer-containing antigen binding molecule according to the invention as described herein. In particular, the isolated nucleic acid encodes a polypeptide comprised in the TNF family ligand trimer-containing antigen binding molecule according to the invention as described herein.

In one aspect, the present invention is directed to isolated nucleic acid encoding a TNF family ligand trimer-containing antigen binding molecule, wherein the nucleic acid comprises (a) a sequence that encodes a first fusion polypeptide as described herein before, (b) a sequence that encodes a second fusion polypeptide as described herein before and optionally (c) a sequence that encodes a light chain.

In certain aspects, the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

TNF family ligand trimer-containing antigen binding molecules of the invention may be produced using recombinant methods and compositions, e.g., as described in U.S. Pat. No. 4,816,567. For recombinant production nucleic acid encoding the TNF family ligand trimer-containing antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising nucleic acid of the invention is provided. Methods which are well known to those skilled in the art can be used to construct expression vectors containing the coding sequence of the TNF family ligand trimer-containing antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the TNF family ligand trimer-containing antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the TNF family ligand trimer-containing antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the TNF family ligand trimer-containing antigen binding molecule or polypeptide fragments thereof is desired, DNA encoding a signal sequence may be placed upstream of the nucleic acid encoding a TNF family ligand trimer-containing antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a TNF family ligand trimer-containing antigen binding molecule of the invention or polypeptide fragments thereof.

In a further aspect of the invention, a host cell comprising nucleic acid of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a TNF family ligand trimer-containing antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, N.J.), pp. 255-268 (2003).

Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one aspect, the host cell is a eukaryotic cell, preferably a mammalian cell, such as a Chinese Hamster Ovary (CHO) cell, a human embryonic kidney (HEK) cell or a lymphoid cell (e.g., YO, NS0, Sp20 cell).

In one aspect, a method of producing a TNF family ligand trimer-containing antigen binding molecule of the invention or polypeptide fragments thereof is provided, wherein the method comprises culturing a host cell comprising nucleic acid encoding the TNF family ligand trimer-containing antigen binding molecule of the invention or polypeptide fragments thereof, as provided herein, under conditions suitable for expression of the TNF family ligand trimer-containing antigen binding molecule of the invention or polypeptide fragments thereof, and recovering the TNF family ligand trimer-containing antigen binding molecule of the invention or polypeptide fragments thereof from the host cell (or host cell culture medium).

TNF ligand trimer-containing antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the TNF ligand trimer-containing antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the TNF ligand trimer-containing antigen binding molecule or fragments thereof can be determined by any of a variety of well-known analytical methods including gel electrophoresis, high pressure liquid chromatography, and the like. For example, the TNF ligand trimer-containing antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art. Biological activity may include, e.g., the ability to enhance the activation and/or proliferation of different immune cells especially T-cells. E.g. they enhance secretion of immunomodulating cytokines (e.g. interferon-gamma (IFNγ) and/or tumor necrosis factor alpha (TNF alpha)). Other immunomodulating cytokines which are or can be enhanced are e.g IL12, Granzyme B etc. Biological activity may also include, cynomolgus binding crossreactivity, as well as binding to different cell types. Antigen binding molecules having such biological activity in vivo and/or in vitro are also provided.

1. Affinity Assays

The affinity of the TNF family ligand trimer-containing antigen binding molecule provided herein for the corresponding TNF receptor can be determined in accordance with the methods set forth in the Examples by surface plasmon resonance (SPR), using standard instrumentation such as a BIACORE® instrument (GE Healthcare), and receptors or target proteins such as may be obtained by recombinant expression. The affinity of the TNF family ligand trimer-containing antigen binding molecule for FAP or CD19 can also be determined by surface plasmon resonance (SPR), using standard instrumentation such as a BIACORE® instrument (GE Healthcare), and receptors or target proteins such as may be obtained by recombinant expression. A specific illustrative and exemplary embodiment for measuring binding affinity is described in Example 4. According to one aspect, K_{d} is measured by surface plasmon resonance using a BIACORE® T100 machine (GE Healthcare) at 25° C.

2. Binding Assays and Other Assays

Binding of the TNF family ligand trimer-containing antigen binding molecule provided herein to the corresponding receptor expressing cells may be evaluated using cell lines expressing the particular receptor or target antigen, for example by flow cytometry (FACS). In one aspect, fresh peripheral blood mononuclear cells (PBMCs) expressing the TNF receptor are used in the binding assay. These cells are used directly after isolation (naïve PMBCs) or after stimulation (activated PMBCs). In another aspect, activated mouse splenocytes (expressing the TNF receptor molecule) were used to demonstrate the binding of the TNF family ligand trimer-containing antigen binding molecule of the invention to the corresponding TNF receptor expressing cells.

In a further aspect, cell lines expressing FAP or CD19 were used to demonstrate the binding of the antigen binding molecules to this target cell antigen.

In another aspect, competition assays may be used to identify an antigen binding molecule that competes with a specific antibody or antigen binding molecule for binding to the target or TNF receptor, respectively. In certain embodiments, such a competing antigen binding molecule binds to the same epitope (e.g., a linear or a conformational epitope) that is bound by a specific anti-target antibody or a specific anti-TNF receptor antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying TNF family ligand trimer-containing antigen binding molecules that bind to a specific target cell antigen and to a specific TNF receptor having biological activity. Biological activity may include, e.g., agonistic signalling through the TNF receptor on cells expressing the target cell antigen. TNF family ligand trimer-containing antigen binding molecules identified by the assays as having such biological activity in vitro are also provided.

In certain aspects, a TNF family ligand trimer-containing antigen binding molecule of the invention is tested for such biological activity. Examples for assays for detecting the biological activity of the molecules of the invention are those described in Examples 4 and 5. The biological activity of can be assessed for example by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

Pharmaceutical Compositions, Formulations and Routes of Administration

In a further aspect, the invention provides pharmaceutical compositions comprising any of the TNF family ligand trimer-containing antigen binding molecules provided herein, e.g., for use in any of the below therapeutic methods. In one embodiment, a pharmaceutical composition comprises any of the TNF family ligand trimer-containing antigen binding molecules provided herein and at least one pharmaceutically acceptable excipient. In another embodiment, a pharmaceutical composition comprises any of the TNF family ligand trimer-containing antigen binding molecules provided herein and at least one additional therapeutic agent, e.g., as described below.

Pharmaceutical compositions of the present invention comprise a therapeutically effective amount of one or more TNF family ligand trimer-containing antigen binding molecules dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one TNF family ligand trimer-containing antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the TNF family ligand trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the fusion proteins may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the fusion proteins of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The TNF family ligand trimer-containing antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Therapeutic Methods and Compositions

Any of the TNF family ligand trimer-containing antigen binding molecules provided herein may be used in therapeutic methods.

For use in therapeutic methods, TNF family ligand trimer-containing antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, TNF family ligand trimer-containing antigen binding molecules of the invention for use as a medicament are provided. In further aspects, TNF family ligand trimer-containing antigen binding molecules of the invention for use in treating a disease, in particular for use in the treatment of cancer, are provided. In certain aspects, TNF family ligand trimer-containing antigen binding molecules of the invention for use in a method of treatment are provided. In one aspect, the invention provides a TNF family ligand trimer-containing antigen binding molecule as described herein for use in the treatment of a disease in an individual in need thereof. In certain aspects, the invention provides a TNF family ligand trimer-containing antigen binding molecule for use in a method of treating an individual having a disease comprising administering to the individual a therapeutically effective amount of the fusion protein. In certain aspects, the disease to be treated is cancer. Examples of cancers include solid tumors, bladder cancer, renal cell carcinoma, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer, melanoma, B-cell lymphoma, B-cell leukemia, non-Hodgkin lymphoma and acute lymphoblastic leukemia. Thus, a TNF family ligand trimer-containing antigen binding molecule as described herein for use in the treatment of cancer is provided. The subject, patient, or “individual” in need of treatment is typically a mammal, more specifically a human.

In another aspect, provided is a TNF family ligand trimer-containing antigen binding molecule as described herein for use in the treatment of infectious diseases, in particular for the treatment of viral infections.

In a further aspect, the invention relates to the use of a TNF family ligand trimer-containing antigen binding molecule in the manufacture or preparation of a medicament for the treatment of a disease in an individual in need thereof. In one aspect, the medicament is for use in a method of treating a disease comprising administering to an individual having the disease a therapeutically effective amount of the medicament. In certain embodiments the disease to be treated is a proliferative disorder, particularly cancer. Thus, in one aspect, the invention relates to the use of a TNF family ligand trimer-containing antigen binding molecule of the invention in the manufacture or preparation of a medicament for the treatment of cancer. Examples of cancers include solid tumors, bladder cancer, renal cell carcinoma, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer, melanoma, B-cell lymphoma, B-cell leukemia, non-Hodgkin lymphoma and acute lymphoblastic leukemia. Other cell proliferation disorders that can be treated using a TNF family ligand trimer-containing antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan may recognize that in some cases the TNF family ligand trimer-containing antigen binding molecule may not provide a cure but may only provide partial benefit. In some aspects, a physiological change having some benefit is also considered therapeutically beneficial. Thus, in some aspects, an amount of TNF family ligand trimer-containing antigen binding molecule that provides a physiological change is considered an “effective amount” or a “therapeutically effective amount”.

In a further aspect, the invention relates to the use of a TNF family ligand trimer-containing antigen binding molecule as described herein in the manufacture or preparation of a medicament for the treatment of infectious diseases, in particular for the treatment of viral infections or for the treatment of autoimmune diseases, for example Lupus disease.

In a further aspect, the invention provides a method for treating a disease in an individual, comprising administering to said individual a therapeutically effective amount of a TNF family ligand trimer-containing antigen binding molecule of the invention. In one aspect a composition is administered to said individual, comprising a fusion protein of the invention in a pharmaceutically acceptable form. In certain aspects, the disease to be treated is a proliferative disorder. In a particular aspect, the disease is cancer. In another aspect, the disease is an infectious disease or an autoimmune disease. In certain aspects, the method further comprises administering to the individual a therapeutically effective amount of at least one additional therapeutic agent, e.g. an anti-cancer agent if the disease to be treated is cancer. An “individual” according to any of the above embodiments may be a mammal, preferably a human.

For the prevention or treatment of disease, the appropriate dosage of a TNF family ligand trimer-containing antigen binding molecule of the invention (when used alone or in combination with one or more other additional therapeutic agents) will depend on the type of disease to be treated, the route of administration, the body weight of the patient, the type of antigen binding molecule, the severity and course of the disease, whether the fusion protein is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The TNF family ligand trimer-containing antigen binding molecule is suitably administered to the patient at one time or over a series of treatments. Depending on the type and severity of the disease, about 1 μg/kg to 15 mg/kg (e.g. 0.1 mg/kg-10 mg/kg) of TNF family ligand trimer-containing antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). An initial higher loading dose, followed by one or more lower doses may be administered. However, other dosage regimens may be useful. The progress of this therapy is easily monitored by conventional techniques and assays.

The TNF family ligand trimer-containing antigen binding molecules of the invention will generally be used in an amount effective to achieve the intended purpose. For use to treat or prevent a disease condition, the TNF family ligand trimer-containing antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the TNF family ligand trimer-containing antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the TNF family ligand trimer-containing antigen binding molecule may not be related to plasma concentration. One skilled in the art will be able to optimize therapeutically effective local dosages without undue experimentation.

A therapeutically effective dose of the TNF family ligand trimer-containing antigen binding molecules described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. TNF family ligand trimer-containing antigen binding molecules that exhibit large therapeutic indices are preferred. In one embodiment, the TNF family ligand trimer-containing antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with fusion proteins of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The TNF family ligand trimer-containing antigen binding molecules of the invention may be administered in combination with one or more other agents in therapy. For instance, a fusion protein of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent.

Such other agents are suitably present in combination in amounts that are effective for the purpose intended. The effective amount of such other agents depends on the amount of fusion protein used, the type of disorder or treatment, and other factors discussed above. The TNF family ligand trimer-containing antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the TNF family ligand trimer-containing antigen binding molecule of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a TNF ligand trimer-containing antigen binding molecule of the invention.

The label or package insert indicates that the composition is used for treating the condition of choice. Moreover, the article of manufacture may comprise (a) a first container with a composition contained therein, wherein the composition comprises a TNF ligand trimer-containing antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences)

| SEQ | | |
|---|---|---|
| ID NO: | Description | Sequence |
| 1 | Human (hu) 4-1BBL (71-254) | REGPELSPDDPAGLLDLRQGMFAQLVAQNVLL |
| | | IDGPLSWYSDPGLAGVSLTGGLSYKEDTKELV |
| | | VAKAGVYYVFFQLELRRVVAGEGSGSVSLALH |
| | | LQPLRSAAGAAALALTVDLPPASSEARNSAFGF |
| | | QGRLLHLSAGQRLGVHLHTEARARHAWQLTQ |
| | | GATVLGLFRVTPEIPAGLPSPRSE |
| 2 | hu 4-1BBL (85-254) | LDLRQGMFAQLVAQNVLLIDGPLSWYSDPGLA |
| | | GVSLTGGLSYKEDTKELVVAKAGVYYVFFQLE |
| | | LRRVVAGEGSGSVSLALHLQPLRSAAGAAALA |
| | | LTVDLPPASSEARNSAFGFQGRLLHLSAGQRLG |
| | | VHLHTEARARHAWQLTQGATVLGLFRVTPEIP |
| | | AGLPSPRSE |
| 3 | hu 4-1BBL (80-254) | DPAGLLDLRQGMFAQLVAQNVLLIDGPLSWYS |
| | | DPGLAGVSLTGGLSYKEDTKELVVAKAGVYY |
| | | VFFQLELRRVVAGEGSGSVSLALHLQPLRSAA |
| | | GAAALALTVDLPPASSEARNSAFGFQGRLLHLS |
| | | AGQRLGVHLHTEARARHAWQLTQGATVLGLF |
| | | RVTPEIPAGLPSPRSE |
| 4 | hu 4-1BBL (52-254) | PWAVSGARASPGSAASPRLREGPELSPDDPAGL |
| | | LDLRQGMFAQLVAQNVLLIDGPLSWYSDPGLA |
| | | GVSLTGGLSYKEDTKELVVAKAGVYYVFFQLE |
| | | LRRVVAGEGSGSVSLALHLQPLRSAAGAAALA |
| | | LTVDLPPASSEARNSAFGFQGRLLHLSAGQRLG |
| | | VHLHTEARARHAWQLTQGATVLGLFRVTPEIP |
| | | AGLPSPRSE |
| 5 | Human (hu) 4-1BBL (71-248) | REGPELSPDDPAGLLDLRQGMFAQLVAQNVLL |
| | | IDGPLSWYSDPGLAGVSLTGGLSYKEDTKELV |
| | | VAKAGVYYVFFQLELRRVVAGEGSGSVSLALH |
| | | LQPLRSAAGAAALALTVDLPPASSEARNSAFGF |
| | | QGRLLHLSAGQRLGVHLHTEARARHAWQLTQ |
| | | GATVLGLFRVTPEIPAGL |
| 6 | hu 4-1BBL (85-248) | LDLRQGMFAQLVAQNVLLIDGPLSWYSDPGLA |
| | | GVSLTGGLSYKEDTKELVVAKAGVYYVFFQLE |
| | | LRRVVAGEGSGSVSLALHLQPLRSAAGAAALA |
| | | LTVDLPPASSEARNSAFGFQGRLLHLSAGQRLG |
| | | VHLHTEARARHAWQLTQGATVLGLFRVTPEIP |
| | | AGL |
| 7 | hu 4-1BBL (80-248) | DPAGLLDLRQGMFAQLVAQNVLLIDGPLSWYS |
| | | DPGLAGVSLTGGLSYKEDTKELVVAKAGVYY |
| | | VFFQLELRRVVAGEGSGSVSLALHLQPLRSAA |
| | | GAAALALTVDLPPASSEARNSAFGFQGRLLHLS |
| | | AGQRLGVHLHTEARARHAWQLTQGATVLGLF |
| | | RVTPEIPAGL |
| 8 | hu 4-1BBL (52-248) | PWAVSGARASPGSAASPRLREGPELSPDDPAGL |
| | | LDLRQGMFAQLVAQNVLLIDGPLSWYSDPGLA |
| | | GVSLTGGLSYKEDTKELVVAKAGVYYVFFQLE |
| | | LRRVVAGEGSGSVSLALHLQPLRSAAGAAALA |
| | | LTVDLPPASSEARNSAFGFQGRLLHLSAGQRLG |
| | | VHLHTEARARHAWQLTQGATVLGLFRVTPEIP |
| | | AGL |
| 9 | FAP(4B9) CDR-H1 | SYAMS |
| 10 | FAP(4B9) CDR-H2 | AIIGSGASTYYADSVKG |
| 11 | FAP(4B9) CDR-H3 | GWFGGFNY |
| 12 | FAP(4B9) CDR-L1 | RASQSVTSSYLA |
| 13 | FAP(4B9) CDR-L2 | VGSRRAT |
| 14 | FAP(4B9) CDR-L3 | QQGIMLPPT |
| 15 | FAP(28H1) CDR-H1 | SHAMS |
| 16 | FAP(28H1) CDR-H2 | AIWASGEQYYADSVKG |
| 17 | FAP(28H1) CDR-H3 | GWLGNFDY |
| 18 | FAP(28H1) CDR-L1 | RASQSVSRSYLA |
| 19 | FAP(28H1) CDR-L2 | GASTRAT |
| 20 | FAP(28H1) CDR-L3 | QQGQVIPPT |
| 21 | FAP(4B9) VH | EVQLLESGGGLVQPGGSLRLSCAASGFTFSSYA |
| | | MSWVRQAPGKGLEWVSAIIGSGASTYYADSVK |
| | | GRFTISRDNSKNTLYLQMNSLRAEDTAVYYCA |
| | | KGWFGGFNYWGQGTLVTVSS |
| 22 | FAP(4B9) VL | EIVLTQSPGTLSLSPGERATLSCRASQSVTSSYL |
| | | AWYQQKPGQAPRLLINVGSRRATGIPDRFSGSG |
| | | SGTDFTLTISRLEPEDFAVYYCQQGIMLPPTFGQ |
| | | GTKVEIK |
| 23 | FAP(28H1) VH | EVQLLESGGGLVQPGGSLRLSCAASGFTFSSHA |
| | | MSWVRQAPGKGLEWVSAIWASGEQYYADSV |
| | | KGRFTISRDNSKNTLYLQMNSLRAEDTAVYYC |
| | | AKGWLGNFDYWGQGTLVTVSS |
| 24 | FAP(28H1) VL | EIVLTQSPGTLSLSPGERATLSCRASQSVSRSYL |
| | | AWYQQKPGQAPRLLIIGASTRATGIPDRFSGSG |
| | | SGTDFTLTISRLEPEDFAVYYCQQGQVIPPTFGQ |
| | | GTKVEIK |
| 25 | CD19 (8B8-2B11) CDR-H1 | DYIMH |
| 26 | CD19 (8B8-2B11) CDR-H2 | YINPYNDGSKYTEKFQG |
| 27 | CD19 (8B8-2B11) CDR-H3 | GTYYYGPQLFDY |
| 28 | CD19 (8B8-2B11) CDR-L1 | KSSQSLETSTGTTYLN |
| 29 | CD19 (8B8-2B11) CDR-L2 | RVSKRFS |
| 30 | CD19 (8B8-2B11) CDR-L3 | LQLLEDPYT |
| 31 | CD19 (8B8-018) CDR-H1 | DYIMH |
| 32 | CD19 (8B8-018) CDR-H2 | YINPYNDGSKYTEKFQG |
| 33 | CD19 (8B8-018) CDR-H3 | GTYYYGSALFDY |
| 34 | CD19 (8B8-018) CDR-L1 | KSSQSLENPNGNTYLN |
| 35 | CD19 (8B8-018) CDR-L2 | RVSKRFS |
| 36 | CD19 (8B8-018) CDR-L3 | LQLTHVPYT |
| 37 | CD19 (8B8-2B11) VH | QVQLVQSGAEVKKPGASVKVSCKASGYTFTD |
| | | YIMHWVRQAPGQGLEWMGYINPYNDGSKYTE |
| | | KFQGRVTMTSDTSISTAYMELSRLRSDDTAVY |
| | | YCARGTYYYGPQLFDYWGQGTTVTVSS |
| 38 | CD19 (8B8-2B11) VL | DIVMTQTPLSLSVTPGQPASISCKSSQSLETSTG |
| | | TTYLNWYLQKPGQSPQLLIYRVSKRFSGVPDRF |
| | | SGSGSGTDFTLKISRVEAEDVGVYYCLQLLEDP |
| | | YTFGQGTKLEIK |
| 39 | CD19 (8B8-018) VH | QVQLVQSGAEVKKPGASVKVSCKASGYTFTD |
| | | YIMHWVRQAPGQGLEWMGYINPYNDGSKYTE |
| | | KFQGRVTMTSDTSISTAYMELSRLRSDDTAVY |
| | | YCARGTYYYGSALFDYWGQGTTVTVSS |
| 40 | CD19 (8B8-018) VL | DIVMTQTPLSLSVTPGQPASISCKSSQSLENPNG |
| | | NTYLNWYLQKPGQSPQLLIYRVSKRFSGVPDR |
| | | FSGSGSGTDFTLKISRVEAEDVGVYYCLQLTHV |
| | | PYTFGQGTKLEIK |
| 41 | Peptide linker G4S | GGGGS |
| 42 | Peptide linker (G4S)2 | GGGGSGGGGS |
| 43 | Peptide linker (SG4)2 | SGGGGSGGGG |
| 44 | Peptide linker | GGGGGSGGGGSSGGGGS |
| 45 | Peptide linker (G4S)3 | GGGGSGGGGSGGGGS |
| 46 | Peptide linker G4(SG4)2 | GGGGSGGGGSGGGG |
| 47 | Peptide linker (G4S)4 | GGGGSGGGGSGGGGSGGGGS |
| 48 | Peptide linker | GSPGSSSSGS |
| 49 | Peptide linker | GSGSGSGS |
| 50 | Peptide linker | GSGSGNGS |
| 51 | Peptide linker | GGSGSGSG |
| 52 | Peptide linker | GGSGSG |
| 53 | Peptide linker | GGSG |
| 54 | Peptide linker | GGSGNGSG |
| 55 | Peptide linker | GGNGSGSG |
| 56 | Peptide linker | GGNGSG |
| 57 | Human (hu) FAP | UniProt no. Q12884 |
| 58 | mouse FAP | UniProt no. P97321 |
| 59 | human CD19 | UniProt no. P15391 |
| 60 | CH2 domain | APELLGGPSV FLFPPKPKDT LMISRTPEVT |
| | | CVWDVSHEDP EVKFNWYVDG VEVHNAKTKP |
| | | REEQESTYRW SVLTVLHQDW LNGKEYKCKV |
| | | SNKALPAPIE KTISKAK |
| 61 | CH3 domain | GQPREPQVYT LPPSRDELTK NQVSLTCLVK |
| | | GFYPSDIAVE WESNGQPENN YKTTPPVLDS |
| | | DGSFFLYSKL TVDKSRWQQG NVFSCSVMHE |
| | | ALHNHYTQKS LSLSPG |
| 62 | CH1 domain | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKKV |
| 63 | hinge region | DKTHTCPXCP with X being S or P |
| 64 | hinge region | HTCPXCP with X being S or P |
| 65 | hinge region | CPXCP with X being S or P |
| 66 | human lymphotoxin-alpha | UniProt no. P01374 |
| 67 | human TNF | UniProt no. P01375 |
| 68 | human lymphotoxin-beta | UniProt no. Q06643 |
| 69 | human OX40L | UniProt no. P23510 |
| 70 | human CD40L | UniProt no. P29965 |
| 71 | human FasL | UniProt no. P48023 |
| 72 | human CD27L | UniProt no. P32970 |
| 73 | human CD30L | UniProt no. P32971 |
| 74 | human 4-1BBL | UniProt no. P41273 |
| 75 | human TRAIL | UniProt no. P50591 |
| 76 | human RANKL | UniProt no. O14788 |
| 77 | human TWEAK | UniProt no. O43508 |
| 78 | human APRIL | UniProt no. O75888 |
| 79 | human BAFF | UniProt no. Q9Y275 |
| 80 | human LIGHT | UniProt no. O43557 |
| 81 | human TL1A | UniProt no. O95150 |
| 82 | human GITRL | UniProt no. Q9UNG2 |
| 83 | human ectodysplasin A | UniProt no. Q92838 |
| 84 | human 4-1BBL(50-254) | ACPWAVSGARASPGSAASPRLREGPELSPDDPA |
| | | GLLDLRQGMFAQLVAQNVLLIDGPLSWYSDPG |
| | | LAGVSLTGGLSYKEDTKELVVAKAGVYYVFFQ |
| | | LELRRVVAGEGSGSVSLALHLQPLRSAAGAAA |
| | | LALTVDLPPASSEARNSAFGFQGRLLHLSAGQR |
| | | LGVHLHTEARARHAWQLTQGATVLGLFRVTP |
| | | EIPAGLPSPRSE |
| 85 | first fusion polypeptide | see Table 1 |
| | (P1AA1199) | |
| 86 | second fusion polypeptide | see Table 1 |
| | (P1AA1199) | |
| 87 | light chain (PlAA1199, | see Table 1 |
| | P1AA1235) | |
| 88 | first fusion polypeptide | see Table 2 |
| | (P1AA1235) | |
| 89 | second fusion polypeptide | see Table 2 |
| | (P1AA1235, P1AA1259) | |
| 90 | first fusion polypeptide | see Table 3 |
| | (P1AA1259) | |
| 91 | second fusion polypeptide | see Table 4 |
| | (P1AA9626) without linker | |
| 92 | second fusion polypeptide | see Table 5 |
| | (with (G4S)2 linker) | |
| 93 | Dimeric hu 4-1BBL (71-248)- | REGPELSPDDPAGLLDLRQGMFAQLVAQNVLL |
| | CL* Fc knob chain | IDGPLSWYSDPGLAGVSLTGGLSYKEDTKELV |
| | (construct 2.4) | VAKAGVYYVFFQLELRRVVAGEGSGSVSLALH |
| | | LQPLRSAAGAAALALTVDLPPASSEARNSAFGF |
| | | QGRLLHLSAGQRLGVHLHTEARARHAWQLTQ |
| | | GATVLGLFRVTPEIPAGLGGGGSGGGGSREGPE |
| | | LSPDDPAGLLDLRQGMFAQLVAQNVLLIDGPL |
| | | SWYSDPGLAGVSLTGGLSYKEDTKELVVAKA |
| | | GVYYVFFQLELRRVVAGEGSGSVSLALHLQPL |
| | | RSAAGAAALALTVDLPPASSEARNSAFGFQGR |
| | | LLHLSAGQRLGVHLHTEARARHAWQLTQGAT |
| | | VLGLFRVTPEIPAGLGGGGSGGGGSRTVAAPSV |
| | | FIFPPSDRKLKSGTASVVCLLNNFYPREAKVQW |
| | | KVDNALQSGNSQESVTEQDSKDSTYSLSSTLTL |
| | | SKADYEKHKVYACEVTHQGLSSPVTKSFNRGE |
| | | CDKTHTCPPCPAPEAAGGPSVFLFPPKPKDTLM |
| | | ISRTPEVTCVVVDVSHEDPEVKFNWYVDGVEV |
| | | HNAKTKPREEQYNSTYRVVSVLTVLHQDWLN |
| | | GKEYKCKVSNKALGAPIEKTISKAKGQPREPQV |
| | | YTLPPCRDELTKNQVSLWCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| 94 | Monomeric hu 4-1BBL (71-248)- | REGPELSPDDPAGLLDLRQGMFAQLVAQNVLL |
| | CH1* (construct 2.4) | IDGPLSWYSDPGLAGVSLTGGLSYKEDTKELV |
| | | VAKAGVYYVFFQLELRRVVAGEGSGSVSLALH |
| | | LQPLRSAAGAAALALTVDLPPASSEARNSAFGF |
| | | QGRLLHLSAGQRLGVHLHTEARARHAWQLTQ |
| | | GATVLGLFRVTPEIPAGLGGGGSGGGGSASTK |
| | | GPSVFPLAPSSKSTSGGTAALGCLVEDYFPEPV |
| | | TVSWNSGALTSGVHTFPAVLQSSGLYSLSSVVT |
| | | VPSSSLGTQTYICNVNHKPSNTKVDEKVEPKSC |
| 95 | anti-PAP (4B9) Pc hole chain | EVQLLESGGGLVQPGGSLRLSCAASGFTFSSYA |
| | (construct 2.4) | MSWVRQAPGKGLEWVSAIIGSGASTYYADSVK |
| | | GRFTISRDNSKNTLYLQMNSLRAEDTAVYYCA |
| | | KGWFGGFNYWGQGTLVTVSSASTKGPSVFPLA |
| | | PSSKSTSGGTAALGCLVKDYFPEPVTVSWNSG |
| | | ALTSGVHTFPAVLQSSGLYSLSSVVTVPSSSLGT |
| | | QTYICNVNHKPSNTKVDKKVEPKSCDKTHTCP |
| | | PCPAPEAAGGPSVFLFPPKPKDTLMISRTPEVTC |
| | | VVVDVSHEDPEVKFNWYVDGVEVHNAKTKPR |
| | | EEQYNSTYRVVSVLTVLHQDWLNGKEYKCKV |
| | | SNKALGAPIEKTISKAKGQPREPQVCTLPPSRDE |
| | | LTKNQVSLSCAVKGFYPSDIAVEWESNGQPEN |
| | | NYKTTPPVLDSDGSFFLVSKLTVDKSRWQQGN |
| | | VFSCSVMHEALHNHYTQKSLSLSPGK |
| 96 | anti-PAP (4B9) light chain | EIVLTQSPGTLSLSPGERATLSCRASQSVTSSYL |
| | (construct 2.4) | AWYQQKPGQAPRLLINVGSRRATGIPDRFSGSG |
| | | SGTDFTLTISRLEPEDFAVYYCQQGIMLPPTFGQ |
| | | GTKVEIKRTVAAPSVFIFPPSDEQLKSGTASVVC |
| | | LLNNFYPREAKVQWKVDNALQSGNSQESVTE |
| | | QDSKDSTYSLSSTLTLSKADYEKHKVYACEVT |
| | | HQGLSSPVTKSFNRGEC |
| 97 | DP47 Pc hole chain | EVQLLESGGGLVQPGGSLRLSCAASGFTFSSYA |
| | | MSWVRQAPGKGLEWVSAISGSGGSTYYADSV |
| | | KGRFTISRDNSKNTLYLQMNSLRAEDTAVYYC |
| | | AKGSGFDYWGQGTLVTVSSASTKGPSVFPLAP |
| | | SSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKKVEPKSCDKTHTCPP |
| | | CPAPEAAGGPSVFLFPPKPKDTLMISRTPEVTCV |
| | | VVDVSHEDPEVKFNWYVDGVEVHNAKTKPRE |
| | | EQYNSTYRVVSVLTVLHQDWLNGKEYKCKVS |
| | | NKALGAPIEKTISKAKGQPREPQVCTLPPSRDEL |
| | | TKNQVSLSCAVKGFYPSDIAVEWESNGQPENN |
| | | YKTTPPVLDSDGSFFLVSKLTVDKSRWQQGNV |
| | | FSCSVMHEALHNHYTQKSLSLSPGK |
| 98 | DP47 light chain | EIVLTQSPGTLSLSPGERATLSCRASQSVSSSYL |
| | | AWYQQKPGQAPRLLIYGASSRATGIPDRFSGSG |
| | | SGTDFTLTISRLEPEDFAVYYCQQYGSSPLTFGQ |
| | | GTKVEIKRTVAAPSVFIFPPSDEQLKSGTASVVC |
| | | LLNNFYPREAKVQWKVDNALQSGNSQESVTE |
| | | QDSKDSTYSLSSTLTLSKADYEKHKVYACEVT |
| | | HQGLSSPVTKSFNRGEC |
| 99 | second fusion polypeptide of | see Table 8 |
| | P1AA1233 | |
| 100 | CD19 (2B11) light chain | see Table 8 |
| 101 | second fusion polypeptide of | see Table 9 |
| | P1AA1258 | |
| 102 | second fusion polypeptide of | see Table 10 |
| | P1AA10776 | |
| 103 | anti-CD19(8B8-2B11) Fc hole | QVQLVQSGAEVKKPGASVKVSCKASGYTFTD |
| | chain | YIMHWVRQAPGQGLEWMGYINPYNDGSKYTE |
| | | KFQGRVTMTSDTSISTAYMELSRLRSDDTAVY |
| | | YCARGTYYYGPQLFDYWGQGTTVTVSSASTK |
| | | GPSVFPLAPSSKSTSGGTAALGCLVKDYFPEPV |
| | | TVSWNSGALTSGVHTFPAVLQSSGLYSLSSVVT |
| | | VPSSSLGTQTYICNVNHKPSNTKVDKKVEPKSC |
| | | DKTHTCPPCPAPEAAGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALGAPIEKTISKAKGQPREPQVC |
| | | TLPPSRDELTKNQVSLSCAVKGFYPSDIAVEWE |
| | | SNGQPENNYKTTPPVLDSDGSFFLVSKLTVDKS |
| | | RWQQGNVFSCSVMHEALHNHYTQKSLSLSPG |
| | | K |
| 104 | anti-CD19(8B8-2B11) light | DIVMTQTPLSLSVTPGQPASISCKSSQSLETSTG |
| | chain | TTYLNWYLQKPGQSPQLLIYRVSKRFSGVPDRF |
| | | SGSGSGTDFTLKISRVEAEDVGVYYCLQLLEDP |
| | | YTFGQGTKLEIKRTVAAPSVFIFPPSDEQLKSGT |
| | | ASVVCLLNNFYPREAKVQWKVDNALQSGNSQ |
| | | ESVTEQDSKDSTYSLSSTLTLSKADYEKHKVYA |
| | | CEVTHQGLSSPVTKSFNRGEC |
| 105 | CH1 connector | EPKSC |
| 106 | IgG1, caucasian allotype | ASTKGPSVFPLAPSSKSTSGGTAALGCLVKDYF |
| | | PEPVTVSWNSGALTSGVHTFPAVLQSSGLYSLS |
| | | SVVTVPSSSLGTQTYICNVNHKPSNTKVDKKVE |
| | | PKSCDKTHTCPPCPAPELLGGPSVFLFPPKPKDT |
| | | LMISRTPEVTCVVVDVSHEDPEVKFNWYVDGV |
| | | EVHNAKTKPREEQYNSTYRVVSVLTVLHQDW |
| | | LNGKEYKCKVSNKALPAPIEKTISKAKGQPREP |
| | | QVYTLPPSRDELTKNQVSLTCLVKGFYPSDIAV |
| | | EWESNGQPENNYKTTPPVLDSDGSFFLYSKLTV |
| | | DKSRWQQGNVFSCSVMHEALHNHYTQKSLSL |
| | | SPGK |
| 107 | IgG1, afroameri can allotype | ASTKGPSVFPLAPSSKSTSGGTAALGCLVKDYF |
| | | PEPVTVSWNSGALTSGVHTFPAVLQSSGLYSLS |
| | | SVVTVPSSSLGTQTYICNVNHKPSNTKVDKKVE |
| | | PKSCDKTHTCPPCPAPELLGGPSVFLFPPKPKDT |
| | | LMISRTPEVTCVVVDVSHEDPEVKFNWYVDGV |
| | | EVHNAKTKPREEQYNSTYRVVSVLTVLHQDW |
| | | LNGKEYKCKVSNKALPAPIEKTISKAKGQPREP |
| | | QVYTLPPSREEMTKNQVSLTCLVKGFYPSDIAV |
| | | EWESNGQPENNYKTTPPVLDSDGSFFLYSKLTV |
| | | DKSRWQQGNVFSCSVMHEALHNHYTQKSLSL |
| | | SPGK |
| 108 | IgG2 | ASTKGPSVFPLAPCSRSTSESTAALGCLVKDYF |
| | | PEPVTVSWNSGALTSGVHTFPAVLQSSGLYSLS |
| | | SVVTVPSSNFGTQTYTCNVDHKPSNTKVDKTV |
| | | ERKCCVECPPCPAPPVAGPSVFLFPPKPKDTLMI |
| | | SRTPEVTCVVVDVSHEDPEVQFNWYVDGVEV |
| | | HNAKTKPREEQFNSTFRVVSVLTVVHQDWLN |
| | | GKEYKCKVSNKGLPAPIEKTISKTKGQPREPQV |
| | | YTLPPSREEMTKNQVSLTCLVKGFYPSDISVEW |
| | | ESNGQPENNYKTTPPMLDSDGSFFLYSKLTVD |
| | | KSRWQQGNVFSCSVMHEALHNHYTQKSLSLSP |
| | | GK |
| 109 | IgG3 | ASTKGPSVFPLAPCSRSTSGGTAALGCLVKDYF |
| | | PEPVTVSWNSGALTSGVHTFPAVLQSSGLYSLS |
| | | SVVTVPSSSLGTQTYTCNVNHKPSNTKVDKRV |
| | | ELKTPLGDTTHTCPRCPEPKSCDTPPPCPRCPEP |
| | | KSCDTPPPCPRCPEPKSCDTPPPCPRCPAPELLG |
| | | GPSVFLFPPKPKDTLMISRTPEVTCVVVDVSHE |
| | | DPEVQFKWYVDGVEVHNAKTKPREEQYNSTF |
| | | RVVSVLTVLHQDWLNGKEYKCKVSNKALPAPI |
| | | EKTISKTKGQPREPQVYTLPPSREEMTKNQVSL |
| | | TCLVKGFYPSDIAVEWESSGQPENNYNTTPPML |
| | | DSDGSFFLYSKLTVDKSRWQQGNIFSCSVMHE |
| | | ALHNRFTQKSLSLSPGK |
| 110 | IgG4 | ASTKGPSVFPLAPCSRSTSESTAALGCLVKDYF |
| | | PEPVTVSWNSGALTSGVHTFPAVLQSSGLYSLS |
| | | SVVTVPSSSLGTKTYTCNVDHKPSNTKVDKRV |
| | | ESKYGPPCPSCPAPEFLGGPSVFLFPPKPKDTLM |
| | | ISRTPEVTCVVVDVSQEDPEVQFNWYVDGVEV |
| | | HNAKTKPREEQFNSTYRVVSVLTVLHQDWLN |
| | | GKEYKCKVSNKGLPSSIEKTISKAKGQPREPQV |
| | | YTLPPSQEEMTKNQVSLTCLVKGFYPSDIAVE |
| | | WESNGQPENNYKTTPPVLDSDGSFFLYSRLTV |
| | | DKSRWQEGNVFSCSVMHEALHNHYTQKSLSLS |
| | | LGK |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the EU numbering systems according to Kabat (Kabat, E. A., et al., Sequences of Proteins of Immunological Interest, 5th ed., Public Health Service, National Institutes of Health, Bethesda, Md. (1991)) as defined above.

The following numbered paragraphs (paras) describe aspects of the present invention:
1. A TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
   the spacer domain is a polypeptide and comprises at least 25 amino acid residues,
   the first ectodomain of a TNF ligand family member or a fragment thereof is fused either directly or via a first peptide linker to the N-terminus of the spacer domain and
   the second ectodomain of said TNF ligand family member or a fragment thereof is fused either directly or via a second peptide linker to the C-terminus of the spacer domain,
(b) a second fusion polypeptide comprising a first part of an antigen binding domain and a spacer domain, wherein
   the spacer domain is a polypeptide and comprises at least 25 amino acid residues, and
   wherein the second part of the antigen binding domain is fused either directly or via a third peptide linker to the C-terminus of the spacer domain or is present in form of a light chain, and
(c) a third ectodomain of said TNF ligand family member or a fragment thereof that is fused either directly or via a fourth peptide linker to
   either the C-terminus of the second ectodomain of said TNF ligand family member in the first fusion polypeptide or to the C-terminus of the spacer domain in the second fusion polypeptide, or
   in case the second part of the antigen binding domain is fused to the C-terminus of the spacer domain of the second fusion protein, to the C-terminus of the second ectodomain of said TNF ligand family member in the first fusion polypeptide,
wherein the spacer domain of the first fusion polypeptide and the spacer domain of the second fusion polypeptide are associated covalently to each other by a disulfide bond.
2. The TNF family ligand trimer-containing antigen binding molecule of para 1, wherein the first part of the antigen binding domain comprises an antibody heavy chain variable domain and the second part of the antigen binding domain comprises an antibody light chain variable domain or vice versa.
3. The TNF family ligand trimer-containing antigen binding molecule of paras 1 or 2, wherein the first part of the antigen binding domain is an antibody heavy chain Fab fragment and the second part of the antigen binding domain is an antibody light chain Fab fragment or vice versa.
4. The TNF family ligand trimer-containing antigen binding molecule of any one of paras 1 to 3, wherein the first part of the antigen binding domain and the second part of the antigen binding domain are associated covalently to each other by a disulfide bond.
5. The TNF family ligand trimer-containing antigen binding molecule of any one of paras 1 to 4, wherein the spacer domain comprises an antibody hinge region or a (C-terminal) fragment thereof and an antibody CH2 domain or a (N-terminal) fragment thereof
6. The TNF family ligand trimer-containing antigen binding molecule of any one of paras 1 to 5, wherein the spacer domain comprises an antibody hinge region or a fragment thereof, an antibody CH2 domain, and an antibody CH3 domain or a fragment thereof
7. The TNF family ligand trimer-containing antigen binding molecule of any one of paras 1 to 6, wherein the spacer domain of the first fusion polypeptide and the spacer domain of the second fusion polypeptide comprise modifications promoting the association of the first and second fusion polypeptide.
8. The TNF family ligand trimer-containing antigen binding molecule of any one of paras 1 to 7, wherein the spacer domain of the first fusion polypeptide comprises holes and the spacer domain of the second fusion polypeptide comprises knobs according to the knobs into hole method.
9. The TNF family ligand trimer-containing antigen binding molecule of any one of paras 1 to 8, wherein the spacer domain comprises an antibody hinge region or a fragment thereof and an IgG1 Fc domain.
10. The TNF family ligand trimer-containing antigen binding molecule of any one of paras 1 to 9, wherein the IgG1 Fc domain comprises amino acid substitutions L234A, L235A and P329G (numbering according to Kabat EU index).
11. The TNF family ligand trimer-containing antigen binding molecule of any one of paras 1 to 10, wherein the TNF ligand family member is 4-1BBL.
12. The TNF family ligand trimer-containing antigen binding molecule of any one of paras 1 to 11, wherein the ectodomain of the TNF ligand family member comprises the amino acid sequence selected from the group consisting of SEQ ID NO: 1, SEQ ID NO: 2, SEQ ID NO:
3, SEQ ID NO: 4, SEQ ID NO: 5, SEQ ID NO: 6, SEQ ID NO: 7 and SEQ ID NO: 8, particularly the amino acid sequence of SEQ ID NO: 1 or SEQ ID NO: 5.
13. The TNF family ligand trimer-containing antigen binding molecule of any one of paras 1 to 12, wherein the antigen binding domain is capable of specific binding to a tumor associated antigen.
14. The TNF family ligand trimer-containing antigen binding molecule of any one of paras 1 to 13, wherein the antigen binding domain is capable of specific binding to Fibroblast Activation Protein (FAP) or CD19.
15. The TNF family ligand trimer-containing antigen binding molecule of any one of paras 1 to 14, wherein the antigen binding domain capable of specific binding to FAP comprises (a) a heavy chain variable region (V_{H}FAP) comprising (i) CDR-H1 comprising the amino acid sequence of SEQ ID NO: 9, (ii) CDR-H2 comprising the amino acid sequence of SEQ ID NO: 10, and (iii) CDR-H3 comprising the amino acid sequence of SEQ ID NO: 11, and a light chain variable region (V_{L}FAP) comprising (iv) CDR-L1 comprising the amino acid sequence of SEQ ID NO: 12, (v) CDR-L2 comprising the amino acid sequence of SEQ ID NO: 13, and (vi) CDR-L3 comprising the amino acid sequence of SEQ ID NO: 14, or (b) a heavy chain variable region (V_{H}FAP) comprising (i) CDR-H1 comprising the amino acid sequence of SEQ ID NO: 15, (ii) CDR-H2 comprising the amino acid sequence of SEQ ID NO: 16, and (iii) CDR-H3 comprising the amino acid sequence of SEQ ID NO: 17, and a a light chain variable region (V_{L}FAP) comprising (iv) CDR-L1 comprising the amino acid sequence of SEQ ID NO: 18, (v) CDR-L2 comprising the amino acid sequence of SEQ ID NO: 19, and (vi) CDR-L3 comprising the amino acid sequence of SEQ ID NO: 20.
16. The TNF family ligand trimer-containing antigen binding molecule of any one of paras 1 to 15, wherein the antigen binding domain capable of specific binding to FAP comprises
   (a) a heavy chain variable region (V_{H}FAP) comprising an amino acid sequence that is at least about 95%, 96%, 97%, 98%, 99% or 100% identical to the amino acid sequence of SEQ ID NO: 21, and a light chain variable region (V_{L}FAP) comprising an amino acid sequence that is at least about 95%, 96%, 97%, 98%, 99% or 100% identical to the amino acid sequence of SEQ ID NO: 22, or
   (b) a heavy chain variable region (V_{H}FAP) comprising an amino acid sequence that is at least about 95%, 96%, 97%, 98%, 99% or 100% identical to the amino acid sequence of SEQ ID NO: 23, and a light chain variable region (V_{L}FAP) comprising an amino acid sequence that is at least about 95%, 96%, 97%, 98%, 99% or 100% identical to the amino acid sequence of SEQ ID NO: 24.
17. The TNF family ligand trimer-containing antigen binding molecule of any one of paras 1 to 14, wherein the antigen binding domain capable of specific binding to CD19 comprises (a) a heavy chain variable region (V_{H}CD19) comprising (i) CDR-H1 comprising the amino acid sequence of SEQ ID NO: 25, (ii) CDR-H2 comprising the amino acid sequence of SEQ ID NO: 26, and (iii) CDR-H3 comprising the amino acid sequence of SEQ ID NO: 27, and a light chain variable region (V_{L}CD19) comprising (iv) CDR-L1 comprising the amino acid sequence of SEQ ID NO: 28, (v) CDR-L2 comprising the amino acid sequence of SEQ ID NO: 29, and (vi) CDR-L3 comprising the amino acid sequence of SEQ ID NO: 30, or (b) a heavy chain variable region (V_{H}CD19) comprising (i) CDR-H1 comprising the amino acid sequence of SEQ ID NO: 31, (ii) CDR-H2 comprising the amino acid sequence of SEQ ID NO: 32, and (iii) CDR-H3 comprising the amino acid sequence of SEQ ID NO: 33, and a a light chain variable region (V_{L}CD19) comprising (iv) CDR-L1 comprising the amino acid sequence of SEQ ID NO: 34, (v) CDR-L2 comprising the amino acid sequence of SEQ ID NO: 35, and (vi) CDR-L3 comprising the amino acid sequence of SEQ ID NO: 36.
18. The TNF family ligand trimer-containing antigen binding molecule of any one of paras 1 to 15, wherein the antigen binding domain capable of specific binding to FAP comprises
   (a) a heavy chain variable region (V_{H}CD19) comprising an amino acid sequence that is at least about 95%, 96%, 97%, 98%, 99% or 100% identical to the amino acid sequence of SEQ ID NO: 37, and a light chain variable region (V_{L}CD19) comprising an amino acid sequence that is at least about 95%, 96%, 97%, 98%, 99% or 100% identical to the amino acid sequence of SEQ ID NO: 38, or
   (b) a heavy chain variable region (V_{H}CD19) comprising an amino acid sequence that is at least about 95%, 96%, 97%, 98%, 99% or 100% identical to the amino acid sequence of SEQ ID NO: 39, and a light chain variable region (V_{L}CD19) comprising an amino acid sequence that is at least about 95%, 96%, 97%, 98%, 99% or 100% identical to the amino acid sequence of SEQ ID NO: 40.
19. The TNF family ligand trimer-containing antigen binding molecule of any one of paras 1 to 18, wherein the first, second, third and fourth peptide linker is present and consists of an amino acid sequence selected from the group consisting of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55 and SEQ ID NO: 56.
20. Isolated nucleic acid encoding the TNF family ligand trimer-containing antigen binding molecule of any one of paras 1 to 19.
21. A host cell comprising the nucleic acid of para 20.
22. A method of producing a TNF family ligand trimer-containing antigen binding molecule comprising culturing the host cell of para 21 under conditions suitable for the expression of the TNF family ligand trimer-containing antigen binding molecule.
23. The method of para 22, further comprising recovering the TNF family ligand trimer-containing antigen binding molecule from the host cell.
24. A pharmaceutical composition comprising the TNF family ligand trimer-containing antigen binding molecule of any one of paras 1 to 19 and a pharmaceutically acceptable excipient.
25. The TNF family ligand trimer-containing antigen binding molecule of any one of paras 1 to 19 or the pharmaceutical composition of para 24 for use as medicament.
26. The TNF family ligand trimer-containing antigen binding molecule of any one of paras 1 to 19 or the pharmaceutical composition of para 24 for use in treating cancer.
27. Use of the TNF family ligand trimer-containing antigen binding molecule of any one of paras 1 to 19 or the pharmaceutical composition of para 24 in the manufacture of a medicament for treating cancer.
28. Use of the TNF family ligand trimer-containing antigen binding molecule of any one of paras 1 to 19 or the pharmaceutical composition of para 24 in the manufacture of a medicament for stimulating an immune response.
29. A method of treating an individual having cancer comprising administering to the individual an effective amount of the TNF family ligand trimer-containing antigen binding molecule of any one of paras 1 to 19 or the pharmaceutical composition of para 24.
30. The method of para 29, further comprising administering an additional therapeutic agent to the individual.
31. A method of stimulating the immune response in an individual having cancer comprising administering to the individual an effective amount of the TNF family ligand trimer-containing antigen binding molecule of any one of paras 1 to 19 or the pharmaceutical composition of para 24.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene and Oligonucleotide Synthesis

Desired gene segments were prepared by chemical synthesis at GeneArt® AG (Regensburg, Germany) from synthetic oligonucleotides by automated gene synthesis. The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Reagents

All commercial chemicals, antibodies and kits were used as provided according to the manufacturer's protocol if not stated otherwise.

### Example 1

### Generation of 4-1BBL Trimer-Containing Antigen Binding Molecules (4-1BBL Contorsbodies)

### 1.1 Construction of the Expression Plasmids for the 4-1BBL Trimer-Containing Antigen Binding Molecules (4-1BBL Contorsbodies)

For the expression of a 4-1BBL trimer-containing antigen binding molecules as reported herein a transcription unit comprising the following functional elements was used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a nucleic acid encoding the respective circular fusion polypeptide, and
the bovine growth hormone polyadenylation sequence (BGH pA).

Beside the expression unit/cassette including the desired gene to be expressed the basic/standard mammalian expression plasmid contains
an origin of replication from the vector pUC18 which allows replication of this plasmid in E. coli, and
a beta-lactamase gene which confers ampicillin resistance in E. coli.

### 1.2 Expression of the 4-1BBL Trimer-Containing Antigen Binding Molecules (4-1BBL Contorsbodies)

Transient expression of 4-1BBL trimer-containing antigen binding molecules was performed in suspension-adapted HEK293F (FreeStyle™ 293-F cells; Invitrogen) cells with 293-Free™ Transfection Reagent (Novagen).

Cells were passaged, by dilution, at least four times (volume 30 ml) after thawing in a 125 ml shake flask (Incubate/Shake at 37° C., 7% CO₂, 85% humidity, 135 rpm). The cells were expanded to 3×10⁵ cells/ml in 250 ml volume. Three days later, cells were split and new seeded with a density of 7×10⁵ cells/ml in a 250 ml volume in a 1 liter shake flask. Transfection will be 24 hours later at a cell density around 1.4-2.0×10⁶ cells/ml.

Before transfection 250 μg plasmid-DNA were diluted in a final volume of 10 ml with pre-heated (water bath; 37° C.) Opti-MEM® (Gibco). The solution was gently mixed and incubated at room temperature for not longer than 5 min. Then 333.3 μl 293-Free™ Transfection Reagent were added to the DNA-OptiMEM® solution. Thereafter the solution was gently mixed and incubated at room temperature for 15-20 minutes. The whole volume of mixture was added to 1 L shake flask with 250 ml HEK-cell-culture-volume.

Incubate/Shake at 37° C., 7% CO₂, 85% humidity, 135 rpm for 6 or 7 days.

The supernatant was harvested by a first centrifugation-step at 2,000 rpm, 4° C., for 10 minutes. Then the supernatant was transferred into a new centrifugation-flask for a second centrifuge at 4,000 rpm, 4° C., for 20 minutes. Thereafter the cell-free-supernatant was filtered through a 0.22 μm bottle-top-filter and stored in a freezer (−20° C.).

### 1.3 Purification of the 4-1BBL Trimer-Containing Antigen Binding Molecules (4-1BBL Contorsbodies)

The antigen binding molecule-containing culture supernatants were filtered and purified by two chromatographic steps. The antibodies were captured by affinity chromatography using HiTrap™ MabSelect SuRe™ (GE Healthcare) equilibrated with PBS (1 mM KH₂PO₄, 10 mM Na₂HPO₄, 137 mM NaCl, 2.7 mM KCl), pH 7.4. Unbound proteins were removed by washing with equilibration buffer, and the antigen binding molecule was recovered with 50 mM citrate buffer, pH 2.8, and immediately after elution neutralized to pH 6.0 with 1 M Tris-base, pH 9.0. Size exclusion chromatography on Superdex 200™ (GE Healthcare) was used as second purification step. The size exclusion chromatography was performed in 20 mM histidine buffer, 0.14 M NaCl, pH 6.0. The 4-1BBL trimer-containing antigen binding molecules containing solutions were concentrated with an Ultrafree-CL centrifugal filter unit equipped with a Biomax®-SK membrane (Millipore, Billerica, Mass.) and stored at −80° C.

### 1.4 Mass Spectrometric Analysis of the 4-1BBL Trimer-Containing Antigen Binding Molecules (4-1BBL Contorsbodies)

PNGase F was obtained from Roche Diagnostics GmbH (14.3 U/μl; solution in sodium phosphate, EDTA and glycerol). A protease specifically cleaving in the hinge region of an IgG antibody was freshly reconstituted from a lyophilisate prior to digestion.

Enzymatic Deglycosylation of with PNGase F

50 μg of antigen binding molecule was diluted to a final concentration of 0.6 mg/ml with 10 mM sodium phosphate buffer, pH 7.1, and deglycosylated with 1 μl PNGase F at 37° C. for 16 hours.

Enzymatic Cleavage

The deglycosylated sample was diluted to a final concentration of 0.5 mg/ml with 200 mM Tris buffer, pH 8.0, and subsequently digested with the IgG specific protease at 37° C. for 1 hour.

ESI-QTOF Mass Spectrometry

The sample was desalted by HPLC on a Sephadex® G25 column (Kronlab, 5×250 mm, TAC05/250G0-SR) using 40% acetonitrile with 2% formic acid (v/v). The total mass was determined via ESI-QTOF MS on the maXis™ 4G UHR-QTOF MS system (Bruker Daltonik) equipped with a TriVersa NanoMate® source (Advion). Calibration was performed with sodium iodide (Waters ToF G2-Sample Kit 2 Part: 700008892-1). For the digested antigen binding molecule, data acquisition was done at 1000-4000 m/z (ISCID: 30 eV). The raw mass spectra were evaluated and transformed into individual relative molar masses. For visualization of the results proprietary software was used to generate deconvoluted mass spectra.

### Example 2

### Preparation of FAP-Targeted 4-1BB Ligand Trimer-Containing Fc Fusion Antigen Binding Molecules (FAP-4-1BBL Contorsbodies)

### 2.1 Preparation of FAP (4B9)-4-1BB Ligand (71-248) Contorsbody P1AA1199

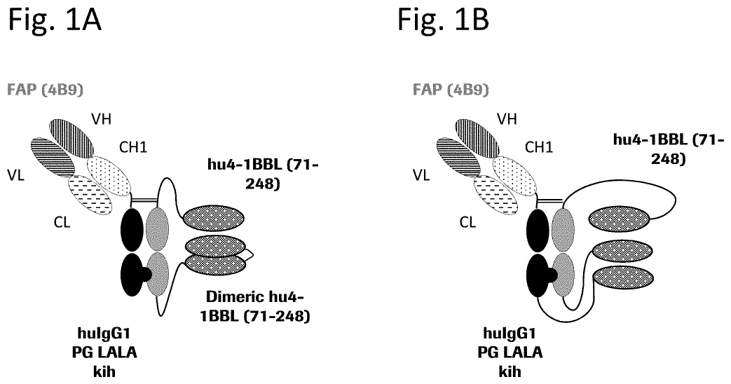

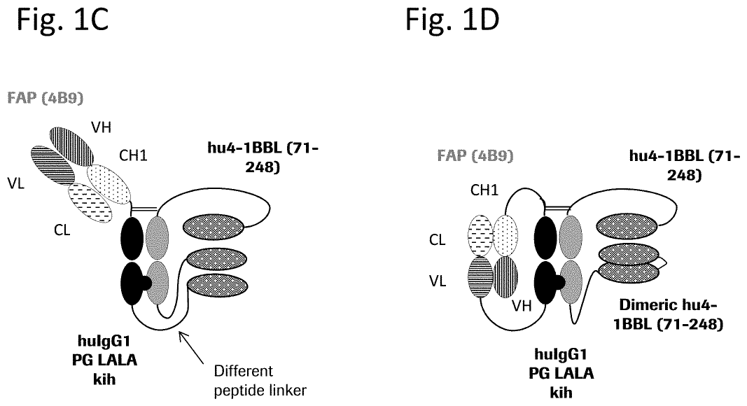

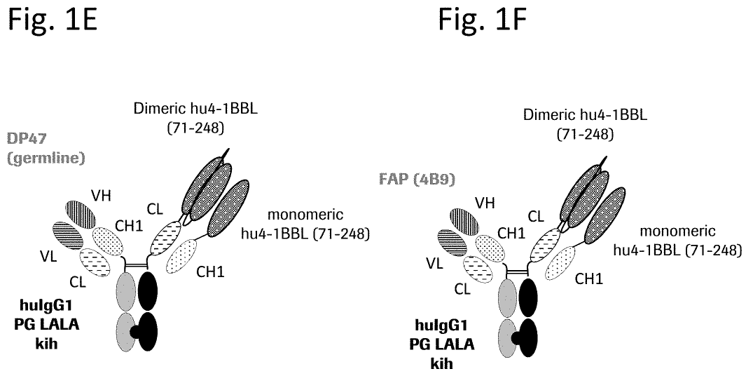

An antigen binding molecule comprising two fusion polypeptides and a light chain was cloned as depicted in FIG. 1A:
first fusion polypeptide (from N- to C-terminus): 4-1BBL(71-248), (G4S)2 connector, IgG1 hinge, Fc hole, (G4S)2 connector, 4-1BBL(71-248), (G4S)2 connector, 4-1BBL(71-248),
second fusion polypeptide (from N- to C-terminus): VH(FAP), CH1, IgG1 hinge, Fc knob, and light chain (from N- to C-terminus): VL(FAP)-Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 1 shows the amino acid sequences of the FAP(4B9)-human 4-1BB ligand (71-248) trimer-containing antigen binding molecule P1AA1199.

TABLE 1
Sequences of P1AA1199

| SEQ ID | | |
|---|---|---|
| NO: | Description | Sequence |
| 85 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGLGGGGSGGGGSREGPELSPDDPAGLLDLRQGMFAQLV |
| | | AQNVLLIDGPLSWYSDPGLAGVSLTGGLSYKEDTKELVVA |
| | | KAGVYYVFFQLELRRVVAGEGSGSVSLALHLQPLRSAAGA |
| | | AALALTVDLPPASSEARNSAFGFQGRLLHLSAGQRLGVHL |
| | | HTEARARHAWQLTQGATVLGLFRVTPEIPAGL |
| 86 | second fusion | EVQLLESGGGLVQPGGSLRLSCAASGFTFSSYAMSWVRQA |
| | poylpeptide | PGKGLEWVSAIIGSGASTYYADSVKGRFTISRDNSKNTLYL |
| | | QMNSLRAEDTAVYYCAKGWFGGFNYWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPG |
| 87 | light chain | EIVLTQSPGTLSLSPGERATLSCRASQSVTSSYLAWYQQKP |
| | | GQAPRLLINVGSRRATGIPDRFSGSGSGTDFTLTISRLEPEDF |
| | | AVYYCQQGIMLPPTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |

### 2.2 Preparation of FAP (4B9)-4-1BB Ligand (71-248) Contorsbody P1AA1235

An antigen binding molecule comprising two fusion polypeptides and a light chain was cloned as depicted in FIG. 1B:
first fusion polypeptide (from N- to C-terminus): 4-1BBL(71-248), (G4S)2 connector, IgG1 hinge, Fc hole, (G4S)2 connector, 4-1BBL(71-248),
second fusion polypeptide (from N- to C-terminus): VH(FAP), CH1, IgG1 hinge, Fc knob, (G4S)2 connector, 4-1BBL(71-248), and light chain (from N- to C-terminus): V_{L}(FAP)-Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 2 shows the amino acid sequences of the FAP(4B9)-human 4-1BB ligand (71-248) trimer-containing antigen binding molecule P1AA1235.

TABLE 2
Sequences of P1AA1235

| SEQ ID | | |
|---|---|---|
| NO: | Description | Sequence |
| 88 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGL |
| 89 | second fusion | EVQLLESGGGLVQPGGSLRLSCAASGFTFSSYAMSWVRQA |
| | poylpeptide | PGKGLEWVSAIIGSGASTYYADSVKGRFTISRDNSKNTLYL |
| | | QMNSLRAEDTAVYYCAKGWFGGFNYWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGGGGGSGGGGSREGPELSPDDPAGLLDLRQGMFAQ |
| | | LVAQNVLLIDGPLSWYSDPGLAGVSLTGGLSYKEDTKELV |
| | | VAKAGVYYVFFQLELRRVVAGEGSGSVSLALHLQPLRSAA |
| | | GAAALALTVDLPPASSEARNSAFGFQGRLLHLSAGQRLGV |
| | | HLHTEARARHAWQLTQGATVLGLFRVTPEIPAGL |
| 87 | light chain | EIVLTQSPGTLSLSPGERATLSCRASQSVTSSYLAWYQQKP |
| | | GQAPRLLINVGSRRATGIPDRFSGSGSGTDFTLTISRLEPEDF |
| | | AVYYCQQGIMLPPTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |

### 2.3 Preparation of FAP (4B9)-4-1BB Ligand (71-248) Contorsbody P1AA1259

An antigen binding molecule comprising two fusion polypeptides and a light chain was cloned as depicted in FIG. 1C:
first fusion polypeptide (from N- to C-terminus): 4-1BBL(71-248), (G4S)2 connector, IgG1 hinge, Fc hole, GGGGSGGGGSSGGGGS (SEQ ID NO: 44) connector, 4-1BBL(71-248),
second fusion polypeptide (from N- to C-terminus): VH(FAP), CH1, IgG1 hinge, Fc knob, (G4S)2 connector, 4-1BBL(71-248), and light chain (from N- to C-terminus): VL(FAP)-Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 3 shows the amino acid sequences of the FAP(4B9)-human 4-1BB ligand (71-248) trimer-containing antigen binding molecule P1AA1259.

TABLE 3
Sequences of P1AA1259

| SEQ ID | | |
|---|---|---|
| NO: | Description | Sequence |
| 90 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSSGGGGSREGPELSPDD |
| | | PAGLLDLRQGMFAQLVAQNVLLIDGPLSWYSDPGLAGVSL |
| | | TGGLSYKEDTKELVVAKAGVYYVFFQLELRRVVAGEGSG |
| | | SVSLALHLQPLRSAAGAAALALTVDLPPASSEARNSAFGFQ |
| | | GRLLHLSAGQRLGVHLHTEARARHAWQLTQGATVLGLFR |
| | | VTPEIPAGL |
| 89 | second fusion | EVQLLESGGGLVQPGGSLRLSCAASGFTFSSYAMSWVRQA |
| | poylpeptide | PGKGLEWVSAIIGSGASTYYADSVKGRFTISRDNSKNTLYL |
| | | QMNSLRAEDTAVYYCAKGWFGGFNYWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGGGGGSGGGGSREGPELSPDDPAGLLDLRQGMFAQ |
| | | LVAQNVLLIDGPLSWYSDPGLAGVSLTGGLSYKEDTKELV |
| | | VAKAGVYYVFFQLELRRVVAGEGSGSVSLALHLQPLRSAA |
| | | GAAALALTVDLPPASSEARNSAFGFQGRLLHLSAGQRLGV |
| | | HLHTEARARHAWQLTQGATVLGLFRVTPEIPAGL |
| 87 | light chain | EIVLTQSPGTLSLSPGERATLSCRASQSVTSSYLAWYQQKP |
| | | GQAPRLLINVGSRRATGIPDRFSGSGSGTDFTLTISRLEPEDF |
| | | AVYYCQQGIMLPPTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |

### 2.4 Preparation of FAP (4B9)-4-1BB Ligand (71-248) Contorsbody P1AA9626

An antigen binding molecule comprising two fusion polypeptides was cloned as depicted in FIG. 1D:
first fusion polypeptide (from N- to C-terminus): 4-1BBL(71-248), (G4S)2 connector, IgG1 hinge, Fc hole, (G4S)2 connector, 4-1BBL(71-248), (G4S)2 connector, 4-1BBL(71-248),
second fusion polypeptide (from N- to C-terminus): VH(FAP), CH1, IgG1 hinge, Fc knob, (G4S)2 connector, VL(FAP), Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 4 shows the amino acid sequences of the FAP(4B9)-human 4-1BB ligand (71-248) trimer-containing antigen binding molecule P1AA9626.

TABLE 4
Sequences of P1AA9626

| SEQ ID | | |
|---|---|---|
| NO: | Description | Sequence |
| 85 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGLGGGGSGGGGSREGPELSPDDPAGLLDLRQGMFAQLV |
| | | AQNVLLIDGPLSWYSDPGLAGVSLTGGLSYKEDTKELVVA |
| | | KAGVYYVFFQLELRRVVAGEGSGSVSLALHLQPLRSAAGA |
| | | AALALTVDLPPASSEARNSAFGFQGRLLHLSAGQRLGVHL |
| | | HTEARARHAWQLTQGATVLGLFRVTPEIPAGL |
| 91 | second fusion | EVQLLESGGGLVQPGGSLRLSCAASGFTFSSYAMSWVRQA |
| | poylpeptide | PGKGLEWVSAIIGSGASTYYADSVKGRFTISRDNSKNTLYL |
| | | QMNSLRAEDTAVYYCAKGWFGGFNYWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGKGGGGSGGGGSEIVLTQSPGTLSLSPGERATLSCR |
| | | ASQSVTSSYLAWYQQKPGQAPRLLINVGSRRATGIPDRFSG |
| | | SGSGTDFTLTISRLEPEDFAVYYCQQGIMLPPTFGQGTKVEI |
| | | KRTVAAPSVFIFPPSDEQLKSGTASVVCLLNNFYPREAKVQ |
| | | WKVDNALQSGNSQESVTEQDSKDSTYSLSSTLTLSKADYE |
| | | KHKVYACEVTHQGLSSPVTKSFNRGEC |

Alternatively, the second fusion polypeptide comprises (from N- to C-terminus): VH(FAP), CH1, (G4S)2 connector, IgG1 hinge, Fc knob, (G4S)2 connector, VL(FAP), Ckappa. The sequences of the corresponding molecule are provided in Table 5.

TABLE 5
Sequences of molecule with additional (G4S)2 connector

| SEQ ID | | |
|---|---|---|
| NO: | Description | Sequence |
| 85 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGLGGGGSGGGGSREGPELSPDDPAGLLDLRQGMFAQLV |
| | | AQNVLLIDGPLSWYSDPGLAGVSLTGGLSYKEDTKELVVA |
| | | KAGVYYVFFQLELRRVVAGEGSGSVSLALHLQPLRSAAGA |
| | | AALALTVDLPPASSEARNSAFGFQGRLLHLSAGQRLGVHL |
| | | HTEARARHAWQLTQGATVLGLFRVTPEIPAGL |
| 92 | second fusion | EVQLLESGGGLVQPGGSLRLSCAASGFTFSSYAMSWVRQA |
| | poylpeptide | PGKGLEWVSAIIGSGASTYYADSVKGRFTISRDNSKNTLYL |
| | | QMNSLRAEDTAVYYCAKGWFGGFNYWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCGGGGSGGGGSDKTHTCPPCP |
| | | APEAAGGPSVFLFPPKPKDTLMISRTPEVTCVVVDVSHEDP |
| | | EVKFNWYVDGVEVHNAKTKPREEQYNSTYRVVSVLTVLH |
| | | QDWLNGKEYKCKVSNKALGAPIEKTISKAKGQPREPQVYT |
| | | LPPCRDELTKNQVSLWCLVKGFYPSDIAVEWESNGQPENN |
| | | YKTTPPVLDSDGSFFLYSKLTVDKSRWQQGNVFSCSVMHE |
| | | ALHNHYTQKSLSLSPGKGGGGSGGGGSEIVLTQSPGTLSLS |
| | | PGERATLSCRASQSVTSSYLAWYQQKPGQAPRLLINVGSR |
| | | RATGIPDRFSGSGSGTDFTLTISRLEPEDFAVYYCQQGIMLP |
| | | PTFGQGTKVEIKRTVAAPSVFIFPPSDEQLKSGTASVVCLLN |
| | | NFYPREAKVQWKVDNALQSGNSQESVTEQDSKDSTYSLSS |
| | | TLTLSKADYEKHKVYACEVTHQGLSSPVTKSFNRGEC |

### 2.5 Biochemical Analysis of the Molecules after Purification

Table 6 summarizes the yield and final monomer content of the FAP (4B9) targeted 4-1BB ligand trimer-containing Fc (kih) fusion antigen binding molecules.

TABLE 6
Biochemical analysis of FAP (4B9) targeted 4-1BB ligand trimer-
containing Fc (kih) fusion antigen binding molecules

| | MW | Monomer | Yield |
|---|---|---|---|
| Construct | [kD] | [%] (SEC) | [mg/l] |
| contorsbody P1AA1199 | 155.3 | 88.0 | 4.8 |
| contorsbody P1AA1235 | 155.2 | 100 | 1.8 |
| contorsbody P1AA1259 | 155.6 | 100 | 1.0 |
| contorsbody P1AA9626 | 191.0 | 100 | 4.3 |

### 2.6 Preparation of FAP-Targeted and Untargeted Human 4-1BB Ligand Trimer-Containing Control Molecules

As positive control construct 2.4 as described in WO 2016/075278, Example 2.1.4, was used. This molecule is a monovalent FAP (4B9) targeted 4-1BB ligand (71-248) trimer-containing Fc (kih) fusion antigen binding molecule containing a CH-CL crossover with charged residues. A polypeptide encoding a dimeric 4-1BB ligand fused to human CL domain was subcloned in frame with the human IgG1 heavy chain CH2 and CH3 domains on the knob (Merchant, Zhu et al., Nature Biotechnol. 1998, 16, 677-681). A polypeptide containing one ectodomain of the 4-1BB ligand was fused to the human IgG1-CH1 domain. In Construct 2.4, in order to improve correct pairing the following mutations were additionally introduced in the crossed CH-CL (charged variant). In the dimeric 4-1BB ligand fused to human CL, E123R and Q124K, in the monomeric 4-1BB ligand fused to human CH1, K147E and K213E.

The variable region of heavy and light chain DNA sequences encoding a binder specific for FAP clone 4B9, were subcloned in frame with either the constant heavy chain of the hole or the constant light chain of human IgG1. The Pro329Gly, Leu234Ala and Leu235Ala mutations have been introduced in the constant region of the knob and hole heavy chains to abrogate binding to Fc gamma receptors according to the method described in WO 2012/130831. Combination of the dimeric ligand-Fc knob chain containing the S354C/T366W mutations, the monomeric CH1 fusion, the targeted anti-FAP-Fc hole chain containing the Y349C/T366S/L368A/Y407V mutations and the anti-FAP light chain allows generation of a heterodimer, which includes an assembled trimeric 4-1BB ligand and a FAP binding Fab (FIG. 1F). An untargeted version has been prepared accordingly by replacing the FAP binder by germline DP47 (FIG. 1E).

TABLE 7
Control molecules used in the experiments

| | Example in WO | |
|---|---|---|
| | 2016/075278 | composed of |
| FAP(4B9)-4-1BBL | Example 2.1.4 | SEQ ID NO: 93, SEQ ID NO: 94 |
| (Charged variant) | (Construct 2.4) | SEQ ID NO: 95 and SEQ ID NO: 96 |
| untargeted DP47-4-1BBL | Example 7.3.1 | SEQ ID NO: 93, SEQ ID NO: 94 |
| | (Control D) | SEQ ID NO: 97 and SEQ ID NO: 98 |

### Example 3

### Preparation of CD19-Targeted 4-1BB Ligand Trimer-Containing Fc Fusion Antigen Binding Molecules (CD19-4-1BBL Contorsbodies)

### 3.1 Preparation of CD19 (2B11)-4-1BB Ligand (71-248) Contorsbody P1AA1233

An antigen binding molecule comprising two fusion polypeptides and a light chain was cloned as depicted in FIG. 1B:
first fusion polypeptide (from N- to C-terminus): 4-1BBL(71-248), (G4S)2 connector, IgG1 hinge, Fc hole, (G4S)2 connector, 4-1BBL(71-248),
second fusion polypeptide (from N- to C-terminus): VH(CD19), CH1, IgG1 hinge, Fc knob, (G4S)2 connector, 4-1BBL(71-248), and light chain (from N- to C-terminus): VL(CD19)-Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 8 shows the amino acid sequences of the CD19(4B9)-human 4-1BB ligand (71-248) trimer-containing antigen binding molecule P1AA1233.

TABLE 8
Sequences of P1AA1233

| SEQ ID | | |
|---|---|---|
| NO: | Description | Sequence |
| 88 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGL |
| 99 | second fusion | QVQLVQSGAEVKKPGASVKVSCKASGYTFTDYIMHWVRQ |
| | poylpeptide | APGQGLEWMGYINPYNDGSKYTEKFQGRVTMTSDTSISTA |
| | | YMELSRLRSDDTAVYYCARGTYYYGPQLFDYWGQGTTVT |
| | | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGGGGGSGGGGSREGPELSPDDPAGLLDLRQ |
| | | GMFAQLVAQNVLLIDGPLSWYSDPGLAGVSLTGGLSYKED |
| | | TKELVVAKAGVYYVFFQLELRRVVAGEGSGSVSLALHLQP |
| | | LRSAAGAAALALTVDLPPASSEARNSAFGFQGRLLHLSAG |
| | | QRLGVHLHTEARARHAWQLTQGATVLGLFRVTPEIPAGL |
| 100 | light chain | DIVMTQTPLSLSVTPGQPASISCKSSQSLETSTGTTYLNWYL |
| | | QKPGQSPQLLIYRVSKRFSGVPDRFSGSGSGTDFTLKISRVE |
| | | AEDVGVYYCLQLLEDPYTFGQGTKLEIKRTVAAPSVFIFPP |
| | | SDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |

### 3.2 Preparation of CD19 (2B11)-4-1BB Ligand (71-248) Contorsbody P1AA1258

An antigen binding molecule comprising two fusion polypeptides and a light chain was cloned as depicted in FIG. 1C:
first fusion polypeptide (from N- to C-terminus): 4-1BBL(71-248), (G4S)2 connector, IgG1 hinge, Fc hole, GGGGSGGGGSSGGGGS (SEQ ID NO:44) connector, 4-1BBL(71-248),
second fusion polypeptide (from N- to C-terminus): VH(CD19), CH1, IgG1 hinge, Fc knob, (G4S)2 connector, 4-1BBL(71-248), and light chain (from N- to C-terminus): V_{L}(CD19)-Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 9 shows the amino acid sequences of the CD19(2B11)-human 4-1BB ligand (71-248) trimer-containing antigen binding molecule P1AA1259.

TABLE 9
Sequences of P1AA1258

| SEQ ID | | |
|---|---|---|
| NO: | Description | Sequence |
| 90 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSSGGGGSREGPELSPDD |
| | | PAGLLDLRQGMFAQLVAQNVLLIDGPLSWYSDPGLAGVSL |
| | | TGGLSYKEDTKELVVAKAGVYYVFFQLELRRVVAGEGSG |
| | | SVSLALHLQPLRSAAGAAALALTVDLPPASSEARNSAFGFQ |
| | | GRLLHLSAGQRLGVHLHTEARARHAWQLTQGATVLGLFR |
| | | VTPEIPAGL |
| 101 | second fusion | QVQLVQSGAEVKKPGASVKVSCKASGYTFTDYIMHWVRQ |
| | poylpeptide | APGQGLEWMGYINPYNDGSKYTEKFQGRVTMTSDTSISTA |
| | | YMELSRLRSDDTAVYYCARGTYYYGPQLFDYWGQGTTVT |
| | | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGGGGGSGGGGSREGPELSPDDPAGLLDLRQ |
| | | GMFAQLVAQNVLLIDGPLSWYSDPGLAGVSLTGGLSYKED |
| | | TKELVVAKAGVYYVFFQLELRRVVAGEGSGSVSLALHLQP |
| | | LRSAAGAAALALTVDLPPASSEARNSAFGFQGRLLHLSAG |
| | | QRLGVHLHTEARARHAWQLTQGATVLGLFRVTPEIPAGL |
| 100 | light chain | DIVMTQTPLSLSVTPGQPASISCKSSQSLETSTGTTYLNWYL |
| | | QKPGQSPQLLIYRVSKRFSGVPDRFSGSGSGTDFTLKISRVE |
| | | AEDVGVYYCLQLLEDPYTFGQGTKLEIKRTVAAPSVFIFPP |
| | | SDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |

### 3.3 Preparation of CD19 (2B11)-4-1BB Ligand (71-248) Contorsbody P1AA0776

An antigen binding molecule comprising two fusion polypeptides and a light chain was cloned as depicted in FIG. 1A:
first fusion polypeptide (from N- to C-terminus): 4-1BBL(71-248), (G4S)2 connector, IgG1 hinge, Fc hole, (G4S)2 connector, 4-1BBL(71-248), (G4S)2 connector, 4-1BBL(71-248),
second fusion polypeptide (from N- to C-terminus): VH(CD19), CH1, IgG1 hinge, Fc knob, and light chain (from N- to C-terminus): VL(CD19)-Ckappa.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831. The knobs into hole heterodimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

Table 10 shows the amino acid sequences of the CD19(2B11)-human 4-1BB ligand (71-248) trimer-containing antigen binding molecule P1AA10776.

TABLE 10
Sequences of P1AA0776

| SEQ ID | | |
|---|---|---|
| NO: | Description | Sequence |
| 85 | first fusion | REGPELSPDDPAGLLDLRQGMFAQLVAQNVLLIDGPLSWY |
| | polypeptide | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSDKTHTCPPCPAP |
| | | EAAGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTLP |
| | | PSRDELTKNQVSLSCAVKGFYPSDIAVEWESNGQPENNYK |
| | | TTPPVLDSDGSFFLVSKLTVDKSRWQQGNVFSCSVMHEAL |
| | | HNHYTQKSLSLSPGGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGLGGGGSGGGGSREGPELSPDDPAGLLDLRQGMFAQLV |
| | | AQNVLLIDGPLSWYSDPGLAGVSLTGGLSYKEDTKELVVA |
| | | KAGVYYVFFQLELRRVVAGEGSGSVSLALHLQPLRSAAGA |
| | | AALALTVDLPPASSEARNSAFGFQGRLLHLSAGQRLGVHL |
| | | HTEARARHAWQLTQGATVLGLFRVTPEIPAGL |
| 102 | second fusion | QVQLVQSGAEVKKPGASVKVSCKASGYTFTDYIMHWVRQ |
| | poylpeptide | APGQGLEWMGYINPYNDGSKYTEKFQGRVTMTSDTSISTA |
| | | YMELSRLRSDDTAVYYCARGTYYYGPQLFDYWGQGTTVT |
| | | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 100 | light chain | DIVMTQTPLSLSVTPGQPASISCKSSQSLETSTGTTYLNWYL |
| | | QKPGQSPQLLIYRVSKRFSGVPDRFSGSGSGTDFTLKISRVE |
| | | AEDVGVYYCLQLLEDPYTFGQGTKLEIKRTVAAPSVFIFPP |
| | | SDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |

### 3.4 Biochemical Analysis of the Molecules after Purification

Table 11 summarizes the yield and final monomer content of the CD19 (2B11) targeted 4-1BB ligand trimer-containing Fc (kih) fusion antigen binding molecules.

TABLE 11
Biochemical analysis of CD19 (2B11) targeted 4-1BB ligand
trimer-containing Fc (kih) fusion antigen binding molecules

| | MW | Monomer | Yield |
|---|---|---|---|
| Construct | [kD] | [%] (SEC) | [mg/l] |
| contorsbody P1AA1233 | 156.9 | 100 | 1.3 |
| contorsbody P1AA1258 | 157.3 | 100 | 2.2 |
| contorsbody P1AA0776 | 157.0 | 93.9 | 3.1 |

### 3.5 Preparation of CD19-Targeted and Untargeted Human 4-1BB Ligand Trimer-Containing Control Molecules

As positive control construct 4.4 as described in WO 2016/075278, Example 7.2.6, was used. This molecule is a monovalent CD19 (2B11) targeted 4-1BB ligand (71-248) trimer-containing Fc (kih) fusion antigen binding molecule containing a CH-CL crossover with charged residues. A polypeptide encoding a dimeric 4-1BB ligand fused to human CL domain was subcloned in frame with the human IgG1 heavy chain CH2 and CH3 domains on the knob (Merchant, Zhu et al., Nature Biotechnol. 1998, 16, 677-681). A polypeptide containing one ectodomain of the 4-1BB ligand was fused to the human IgG1-CH1 domain. In Construct 2.4, in order to improve correct pairing the following mutations were additionally introduced in the crossed CH-CL (charged variant). In the dimeric 4-1BB ligand fused to human CL, E123R and Q124K, in the monomeric 4-1BB ligand fused to human CH1, K147E and K213E.

The variable region of heavy and light chain DNA sequences encoding a binder specific for CD19 clone 8B8-2B11, were subcloned in frame with either the constant heavy chain of the hole or the constant light chain of human IgG1. The Pro329Gly, Leu234Ala and Leu235Ala mutations have been introduced in the constant region of the knob and hole heavy chains to abrogate binding to Fc gamma receptors according to the method described in WO 2012/130831. Combination of the dimeric ligand-Fc knob chain containing the S354C/T366W mutations, the monomeric CH1 fusion, the targeted anti-CD19-Fc hole chain containing the Y349C/T366S/L368A/Y407V mutations and the anti-CD19 light chain allows generation of a heterodimer, which includes an assembled trimeric 4-1BB ligand and a FAP binding Fab (FIG. 1F). An untargeted version has been prepared accordingly by replacing the FAP binder by germline DP47 (FIG. 1E).

TABLE 12
Control molecules used in the experiments

| | Example in WO | |
|---|---|---|
| | 2016/075278 | composed of |
| CD19(2B11)-4-1BBL | Example 7.2.6 | SEQ ID NO: 93, SEQ ID NO: 94 |
| (Charged variant) | (Construct 4.4) | SEQ ID NO: 103 and SEQ ID NO: 104 |
| untargeted DP47-4-1BBL | Example 7.3.1 | SEQ ID NO: 93, SEQ ID NO: 94 |
| | (Control D) | SEQ ID NO: 97 and SEQ ID NO: 98 |

### Example 4

### Functional Properties of FAP-Targeted 4-1BBL Trimer-Containing Antigen Binding Molecules of the Invention

### 4.1 HeLa Cells Expressing Human 4-1BB and Reporter Gene NFicB-Luciferase

Agonistic binding of 4-1BB to its ligand induces downstream signaling via activation of nuclear factor kappa B (NFκB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bcl-x(L) and Bfl-1. J Immunol 2002; 169:4882-4888). The recombinant reporter cell line HeLa-hu4-1BB-NFκB-luc clone 26was generated to express human 4-1BB on its surface. Additionally, it harbors a luciferase gene under the control of an NFκB-sensitive enhancer segment allowing the monitoring of 4-1BB activation in a fast and easy manner. 4-1BB triggering induces dose-dependent activation of NFκB, which translocates in the nucleus, where it binds on the NFκB sensitive enhancer of the reporter plasmid to increase expression of the luciferase protein. Luciferase catalyzes luciferin-oxidation resulting in oxyluciferin which emits light. This can be quantified by a luminometer.

Thus, the capacity of the various 4-1BBL containing molecules to induce NFκB activation in HeLa-hu4-1BB-NFκB-luc clone 26 reporter cells was analyzed as a measure for bioactivity. We tested the NFκB activating capacity of different FAP-targeted 4-1BBL antigen binding molecule based molecules versus construct 2.4 and its control D which has been described before (WO 2016/075278). All tested FAP-targeted 4-1BBL antigen binding molecule based molecules were incubated at different concentrations together with reporter cell line HeLa hu4-1BB_NFκB_Luc clone 26 cell line in the absence or absence of FAP-expressing cells mediating hyper-crosslinking. As FAP-expressing cells either the human melanoma cell line WM-266-4 (ATCC® CRL-1676) or NIH/3T3-huFAP clone 19, a mouse embryonic fibroblast NIH/3T3 cell line (ATCC® CRL-1658) transfected with human fibroblast activating protein (huFAP), were added.

Adherent HeLa_hu4-1BB_NFκB_Luc clone 26 cells were cultured over night at a cell density of 0.2×10⁵ cells per well in tissue-culture treated white flat bottom 96-well plates in assay medium (DMEM medium supplied with 10% FBS and 1% GlutaMAX™-I). The next day titrated FAP-targeted 4-1BBL antigen binding molecules (four different contorsbodies, construct 2.4 and control D) were added in the absence of presence of FAP-expressing cells WM-266-4 or NIH/3T3-huFAP clone 19 in a ratio 1:5 between reporter cell line and FAP-expressing cells.

After incubation, assay supernatant was aspirated and plates washed with DPBS. Quantification of light emission was done using the luciferase 100 assay system and the reporter lysis buffer (both Promega, Cat. No. E4550 and Cat-No: E3971) according to manufacturer instructions. Briefly, cells were lysed over night at −20° C. by addition of 50 uL per well 1× lysis buffer. Cells were thawed for 20 minutes at 37° C. before 100 uL per well provided luciferase assay reagent was added. Light emission was quantified immediately with a SpectraMax® M5/M5e microplate reader (Molecular Devices, USA) using 500 ms integration time, without any filter to collect all wavelengths. Emitted relative light units (URL) were corrected by basal luminescence of HeLa_hu4-1BB_NFkB_Luc clone 26 cells and were blotted against the logarithmic primary antibody concentration using Prism4 (GraphPad Software, USA). Curves were fitted using the inbuilt sigmoidal dose response (four parameters, robust fit).

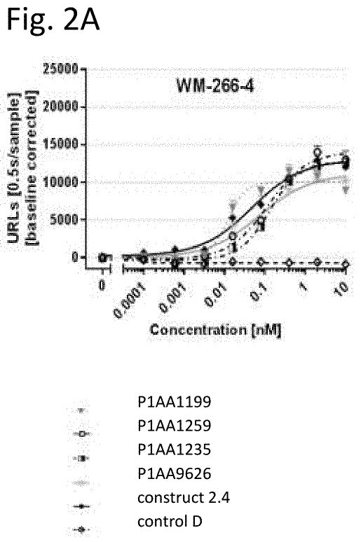

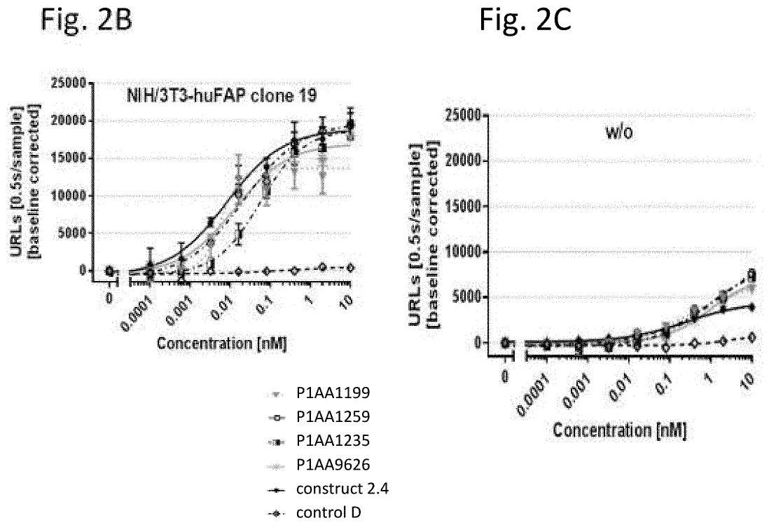

The activities as measured for the tested molecules are shown in FIGS. 2A to 2C and the corresponding measured EC₅₀ values (top) in nM and area under curve of activation curves (bottom) are provided in Table 13 below.

TABLE 13
Measured EC₅₀ values (top) in nM and area under curve of activation curves
(bottom). Shown are calculated means.

| | Construct | | P1AA1235 | P1AA1259 | | |
|---|---|---|---|---|---|---|
| | 2.4 | P1AA1199 | (G4S)2 | (G4S)3 | P1AA9626 | Control D |
| EC₅₀ [nM] | | | | | | |
| no cells | 0.25 | 0.29 | 0.84 | 2.81 | 0.92 | n.d. |
| WM-266-4 | 0.05 | 0.01 | 0.13 | 0.12 | 0.07 | n.d. |
| NIH/3T3-huFAP | 0.01 | 0.01 | 0.06 | 0.02 | 0.01 | n.d. |
| clone 19 | | | | | | |
| AUC | | | | | | |
| no cells | 8030 | 10427 | 10333 | 10520 | 8252 | 1691 |
| WM-266-4 | 31484 | 29738 | 24624 | 27915 | 24209 | 3424 |
| NIH/3T3-huFAP | 56174 | 43026 | 41145 | 49944 | 47289 | 1944 |
| clone 19 | | | | | | |

As shown in FIGS. 2A to 2C, the presence of all FAP-targeted 4-1BBL trimer-containing antigen binding molecules (construct 2.4 and contorsbodies) induced NFκB activation in the human 4-1BB-expressing reporter cell line HeLa-hu4-1BB-NFκB-luc clone 26. Hyper-crosslinking via FAP-expressing cells (WM-266-4 or NIH/3T3-huFAP) increased NFκB activation for all molecules in a FAP independent manner. In the absence of FAP-expressing cells (FIG. 2C) a baseline activity induced by FAP-targeted 4-1BBL (construct 2.4 and contorsbodies) but not by adding untargeted 4-1BBL (control D) was seen. This can be explained by a certain baseline expression of FAP by the HeLa-hu4-1BB-NFκB-luc clone 26 reporter cell line. All constructs showed a concentration-dependent activity in the presence of WM-266-4 or NIH/3T3-huFAP cells reaching the activation-curve plateau around 0.5-1 nM. EC₅₀ values are between 0.01 to 0.13 nM in the presence of FAP-expressing cells (Table 13). No significant differences could be observed between the different FAP-targeted 4-1BBL constructs, only P1AA1199 contorbody showed the tendency towards a lower EC₅₀ and a lower maximum plateau value. Due to the lower EC₅₀ value this difference in plateau did not reduce the area under the curve values in a significant way (Table 13).

### 4.2 FAP-Targeted 4-1BBL Mediated Co-Stimulation of Sub-Optimally TCR Triggered Resting Human PBMC and Hyper-Crosslinking by Cell Surface FAP

It was shown in Example 4.1 that addition of FAP⁺ tumor cells can strongly increase the NFκB activity induced by FAP-targeted 4-1BBL antigen binding molecules (construct 2.4 and molecules of the present application) in human 4-1BB positive reporter cell lines by providing strong oligomerization of 4-1BB receptors. Likewise, we tested FAP-targeted 4-1BBL (construct 2.4 and molecules of the present application) in the presence of NIH/3T3-huFAP clone 19 cells for their ability to promote and increase suboptimal CD3-stimulation of resting human PBMC cells.

Human PBMC preparations contain (1) resting 4-1BB negative CD4⁺ and CD8⁺ T cells and (2) antigen presenting cells with various Fcγ receptor molecules on their cell surface e.g. B cells and monocytes. Anti-human CD3 antibody of human IgG1 isotype can bind with its Fc part to the present Fcγ receptor molecules and mediate a prolonged CD3 activation on resting 4-1BB negative CD4⁺ and CD8⁺ T cells. These T cells then start to express 4-1BB within several hours. Functional agonistic compounds against 4-1BB can signal via the 4-1BB receptor present on activated CD8⁺ and CD4⁺ T cells and support TCR-mediated stimulation.

Resting CFSE-labeled human PBMC were stimulated for five days with a suboptimal concentration of anti-CD3 antibody in the presence of irradiated FAP⁺ NIH/3T3-huFAP clone 19 cells and titrated FAP-targeted 4-1BBL molecules. Effects on T-cells such as proliferation (CFSE-dilution), CD25 and 4-1BB (CD137) were monitored using fluorescently-labeled antibodies and flow cytometry.

Mouse embryonic fibroblast NIH/3T3-huFAP clone 19 cells were harvested using cell dissociation buffer (Invitrogen, Cat.-No. 13151-014) for 10 minutes at 37° C. Cells were washed once with DPBS. 50 Gy irradiated (xRay irradiator) NIH/3T3-huFAP clone 19 cells were cultured at a density of 0.2×10⁵ cells per well in T cell media in a sterile 96-well round bottom adhesion tissue culture plate (TPP, Cat. No 92097) over night at 37° C. and 5% CO₂ in an incubator (Heracell™ 150). The xRay irradiation of NIH/3T3-huFAP clone 19 prevents later overgrowth of human PBMC by the fibroblast cell line.

Human PBMCs were isolated by ficoll density centrifugation. Cells were added to each well at a density of 0.75×10⁵ cells per well. Anti-human CD3 antibody (clone V9, human IgG1) at a final concentration of [2 nM] and FAP-targeted 4-1BBL antigen binding molecules (four different contorsbodies, construct 2.4 and control D) were added at the indicated concentrations. Cells were activated for four days at 37° C. and 5% CO₂ in an incubator (Heracell™ 150).

Then, cells were surface-stained with LIVE/DEAD® Fixable Aqua Dead cell stain (Molecular Probes, Cat.-No. L34957) in DPBS for 30 min at 4° C. in the dark. After washing cells with DPBS, cells were further incubated in PBS supplied with 2% FBS and 5 mM EDTA (FACS-buffer) and fluorescent dye-conjugated antibodies anti-human CD4-BV421 (clone RPA-T4, BioLegend, Cat.-No. 300532), CD8-APC-Cy7 (clone RPA-T8, BioLegend, Cat.-No. 301016), CD25-APC (clone BC96, BioLegend, Cat.-No. 3302610) and CD137 (4-1BB)-PerCP-Cy5.5 (clone 4B4-1, BioLegend, Cat.-No. 309814) for 30 min at 4° C. in the dark. Cells were washed twice with DPBS and staining was fixed with 4% PFA in DPBS. Plates were finally resuspended in 100 μL/well FACS-buffer and acquired using MACSQuant® Analyzer 10 coupled to a Cytomat™ (ThermoFisher). Flow Cytometry data was analyzed using FlowJo™ v10 (FlowJo LLC, USA) and Prism4 (GraphPad Software, USA). Curves were fitted using the inbuilt sigmoidal dose response (four parameters, robust fit).

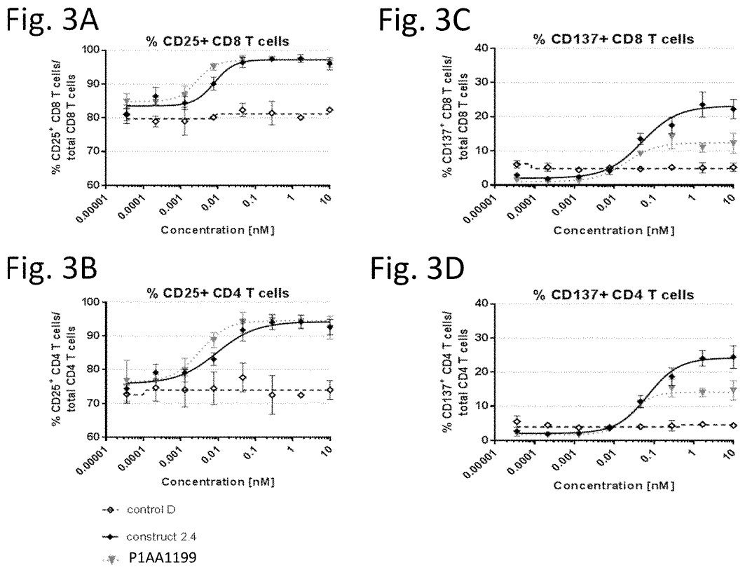

In FIGS. 3A and 3B the upregulation of surface expressed low affinity IL-2-receptor a chain CD25 caused by the FAP-targeted 4-1BBL antigen binding molecules as percentage of positive cells in the CD8⁺ T cells (FIG. 3A) and CD4⁺ T cell population (FIG. 3B) is shown.

The effect on the expression of 4-1BB (CD137) on the cell surface as shown as percentage of positive cells in the CD8⁺ T cells and CD4⁺ T cell population is shown in FIGS. 3C and 3D, respectively. The corresponding measured EC₅₀ values (top) in nM and area under curve of activation curves (bottom) are provided in Table 14 below.

TABLE 14
Measured EC₅₀ values (top) in nM and area under curve
of activation curves (bottom). Shown are calculated means.

| | Control D | Construct 2.4 | P1AA1199 |
|---|---|---|---|
| EC₅₀ [nM] | | | |
| % CD25 + CD8 | n.d. | 0.008 | 0.003 |
| % CD25 + CD4 | n.d. | 0.009 | 0.003 |
| % CD137 + CD8 | n.d. | 0.050 | 0.020 |
| % CD137 + CD4 | n.d. | 0.070 | 0.025 |
| AUC | | | |
| % CD25 + CD8 | 438 | 498 | 505 |
| % CD25 + CD4 | 404 | 471 | 477 |
| % CD137 + CD8 | 27 | 59 | 37 |
| % CD137 + CD4 | 23 | 59 | 43 |

As shown in FIGS. 3A and 3B, co-stimulation with the non-targeted 4-1BBL antigen binding molecule control D (open black diamonds, dotted line) did not rescue sub-optimally TCR stimulated CD4 and CD8 T cells. Hyper-crosslinking of the FAP-targeted 4-1BBL antigen binding molecules (construct 2.4 or contorsbody P1AA1199) by the presence of NIH/3T3-huFAP clone 19 cells strongly promoted an enhanced activated phenotype in human CD4 and CD8 T cells shown as increased expression of CD25 and CD137 (4-1BB). However, contorsbody P1AA1199 induced an increased CD25 expression on CD4 and CD8 T cells with lower EC₅₀ values. On the other hand, contorsbody P1AA1199 displayed a lower frequency of 4-1BB (CD137) expression on CD8 and CD4 T cells (FIGS. 3C and 3D). This may reflect a different T cell activation kinetic or potency compared to construct 2.4. Differences in T cell proliferation were not seen (not shown).

4.3 Summary of Results

It could be shown that FAP-targeted 4-1BBL antigen binding molecules of the invention showed a similar activation potential as the previous described construct 2.4 and are therefore functional. P1AA1199 showed slightly different activation properties displayed in two different functional assays. In the HeLa-hu4-1BB-NFκB-luc reporter cell line P1AA1199 displayed especially in the presence of WM-266-4 a lower EC₅₀ value of dose-dependent NFκB-luciferase activation (FIG. 2A) as well as a lower maximum plateau activation. Both differences were only a tendency and had hardly an effect of total area under the curve of the activation curve (Table 13). In the activation assay with resting human PBMCs P1AA1199 again displayed differences in activity. Dose-dependent increase of CD25 expression on CD4 and CD8 T cells showed a lower EC₅₀ value if induced by adding P1AA1199 compared to construct 2.4. On the other hand increased 4-1BB (CD137) expression on CD4 and CD8 T cells was lower in percentage if induced by adding P1AA1199 compared to construct 2.4. The differences could be explained by a different activity potential or a different T cell activation kinetic.

### Example 5

### Functional Properties of CD19-Targeted 4-1BBL Trimer-Containing Antigen Binding Molecules of the Invention

### 5.1 CD19-4-1BBL Contorsbody Binds to CD19

The binding properties of CD19-4-1BBL contorsbodies P1AA1233, P1AA1258 and P1AA0776 to CD19 was measured on primary human B cells. Briefly, total PBMCs were purified from buffycoats from healthy donors. Cells resuspended in DPBS (Gibco by Life Technologies, Cat. No. 14190 326) were added to each well of a round-bottom suspension cell 96-well plates (greiner bio-one, cellstar, Cat. No. 650185). Cells were washed once with 200 μL DPBS. Cells were resuspended in 100 μL/well of 4° C. cold DPBS buffer containing 1:5000 diluted Fixable Viability Dye eFluor™ 660 (eBioscience, Cat. No. 65-0864-18) and plates were incubated for 30 minutes at 4° C. Cells were washed once with 200 μL/well 4° C. cold DPBS buffer and resuspended in 50 μL/well of 4° C. cold FACS buffer (DPBS supplied with 2% FBS, 5 mM EDTA pH8 (Amresco, Cat. No. E177) and 7.5 mM Sodium azide (Sigma-Aldrich S2002)) containing the constructs (CD19-4-1BBL contorsbody P1AA1233, P1AA1258 and P1AA0776) at a series of concentrations, followed by incubation for 1 hour at 4° C. The controls were construct 4.4 from WO 2016/075278 (CD19-4-1BBL Ab) or the control D (untargeted-4-1BBL Ab, see Example 3.5). After extensive washing, cells were further stained with 50 μL/well of 4° C. cold FACS buffer containing 5 μg/mL PE-conjugated AffiniPure anti-human IgG F(ab′)2-fragment-specific goat F(ab′)2 fragment (Jackson ImmunoResearch, Cat. No. 109 116 098), and with an APC-H7-conjugated CD20 Ab (BD, Cat. No. 560734), and a APC-conjugated anti-CD3 Ab (Biolegend, Cat. No. 300312), and/or a FITC-conjugated anti-CD19 Ab (BD) for 30 minutes at 4° C. Cells were washed twice with 200 μL/well 4° C. FACS buffer and cells were fixed in 50 μL/well DPBS containing 1% Formaldehyde (Sigma, HT501320-9.5L). Cells were resuspended in 100 μL/well FACS-buffer and acquired using the FACS LSR II (BD Biosciences). Data were analyzed using FlowJo™ V10 (FlowJo, LLC) and GraphPad Prism 6.04 (GraphPad Software, Inc).

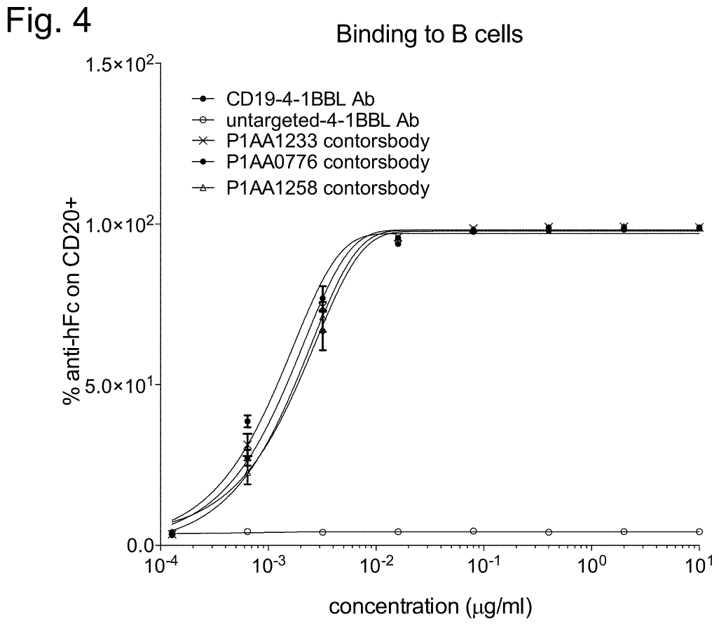

Cells were gated on CD3-CD20⁺ living populations, and geo means of fluorescence intensity of PE-conjugated AffiniPure anti-human IgG IgG Fcγ-fragment-specific goat F(ab′)₂ fragment were plotted against the titrated concentration of constructs. As shown in FIG. 4, all the contorsbodies bind to human B cells in a dose-dependent manner, in a similar pattern as the CD19-4-1BBL Ab (construct 4.4), while untargeted-4-1BBL (control D) did not bind to B cells. These data indicate that the CD19-4-1BBL contorsbodies show specific binding to CD19.

### 5.2 CD19-4-1BBL Contorsbody Binds to 4-1BB on Activated T Cells and NK Cells

To check the binding of CD19-4-1BBL contorsbodies P1AA1233, P1AA1258 and P1AA0776 to 4-1BB expressing T cells or NK cells, human PBMCs were pre-activated by TCR stimulation for the upregulation of 4-1BB on T cells and NK cells for 48 hours. Purified PBMCs were diluted into a concentration of 2.8×10⁶/ml, resuspended in RPMI medium (Gibco, Cat No. 72400-054)+10% FBS (Gibco, Cat No. 20012-068) and 1% penicillin-Streptomycin (Gibco, Cat No. 15070-063) and 50 μM of 2-Mercaptoethanol (Gibco, Cat No. 31350-010). 90 μl of cells was added to each well of a round-bottom 96-well plates (greiner bio-one, cellstar, Cat. No. 650185). Then additional 50 μl anti-CD3 and anti-CD28 microbeads (Life Technologies, Cat No. 11131D) at 8×10⁵ beads/ml were added to the wells. Two days later, cells were washed with cold PBS (Gibco, 20012-068) one time, and resuspended with 90 μl of cold PBS, and incubated with 10 μl of solution containing the CD19-targeted 4-1BBL antigen binding molecules (CD19-4-1BBL contorsbodies P1AA1233, P1AA1258 and P1AA0776, construct 4.4, untargeted control D) for one hour at 4° C. After extensive washing, cells were further stained with 50 μL/well of cold FACS buffer containing 5 μg/mL PE-conjugated AffiniPure anti-human IgG F(ab′)₂-fragment-specific goat F(ab′)₂ fragment (Jackson ImmunoResearch, Cat. No. 109 116 098), and additionally with anti-human CD3 (Biolegend, Cat No. 300312), CD4 (Biolegend, Cat No. 317434), CD8 (Biolegend, Cat No. 344710), CD56 (Biolegend, Cat No. 362504) antibody for 30 minutes at 4° C. Cells were washed twice with 200 μL/well 4° C. cold FACS buffer and cells were fixed in 50 μL/well DPBS containing 1% formaldehyde (Sigma, HT501320-9.5L). Cells were resuspended in 100 μL/well FACS-buffer and acquired using the FACS LSR II (BD Biosciences). Data was analyzed using FlowJo™ V10 (FlowJo, LLC) and GraphPad Prism 6.04 (GraphPad Software, Inc).

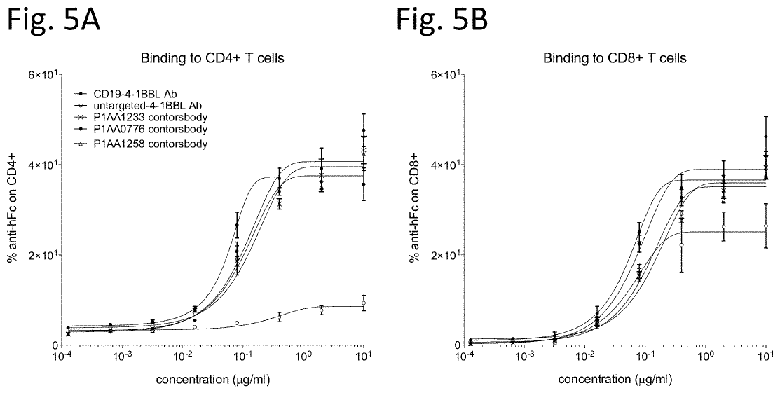

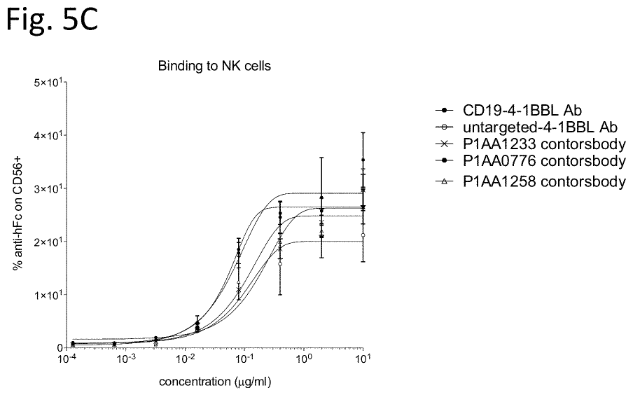

The specific binding was gated on pure population of CD4⁺ and CD8⁺ T cells, and CD56⁺ NK cells. As can be seen in FIGS. 5A, 5B and 5C, respectively, CD19-4-1BBL contorsbodies showed excellent binding to 4-1BB expressing CD4⁺, CD8⁺ T cells and CD56⁺ NK cells in a dose dependent manner, similar to the binding affinity by CD19-4-1BBL Ab (construct 4.4).

### 5.3 CD19-4-1BBL Contorsbody Shows Biological Activity

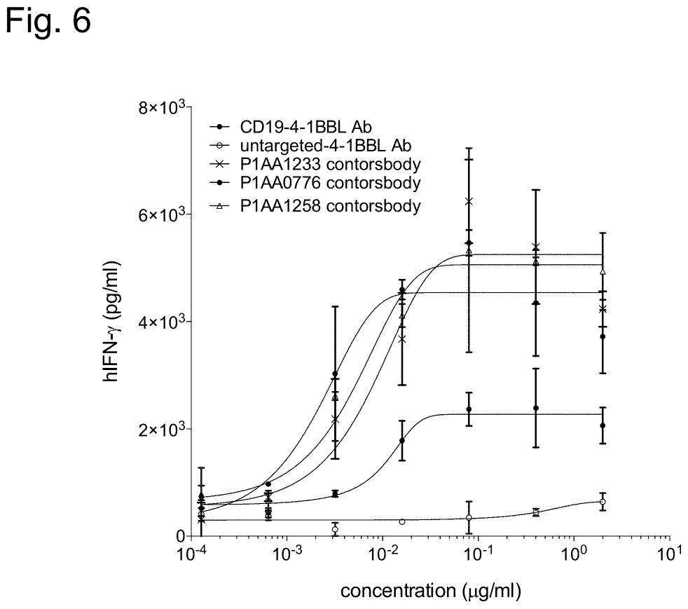

To measure the biological activities in physiological settings, we used activated human PBMCs to check the release of effector function molecule IFNγ by costimluating T cells and NK cells with CD19-4-1BBL contorsbodies. Briefly, purified PBMCs co-cultured with CD19-4-1BBL contorsbodies P1AA1233, P1AA1258 and P1AA0776 were added to the wells at a series of concentrations, and provided with additional 50 μl of anti-CD3 and anti-CD28 microbeads (Life Technologies, Cat No. 11131D) at 8×10⁵ beads/ml. After 48 hours of incubation, the supernatants were collected for the measurement of IFN-γ by ELISA (DuoSet Human IFNg ELISA kit, R&D Systems, Cat No. DY285). FIG. 6 shows that both CD19-4-1BBL contorsbodies P1AA1233 and P1AA1258 stimulate PBMCs to produce a similar amount of IFNγ in a dose dependent manner as induced by the CD19-4-1BBL Ab (construct 4.4), whereas the untargeted 4-1BBL construct (negative control D) did not activate T or NK cells due to the lack of cross-linking. Among those, construct P1AA0776 was less potent to activate 4-1BB⁺ T cells and NK cells.

1. A TNF family ligand trimer-containing antigen binding molecule comprising
(a) a first fusion polypeptide comprising a first ectodomain of a TNF ligand family member or a fragment thereof, a spacer domain and a second ectodomain of said TNF ligand family member or a fragment thereof, wherein
the spacer domain is a polypeptide and comprises at least 25 amino acid residues,
the first ectodomain of a TNF ligand family member or a fragment thereof is fused either directly or via a first peptide linker to the N-terminus of the spacer domain and
the second ectodomain of said TNF ligand family member or a fragment thereof is fused either directly or via a second peptide linker to the C-terminus of the spacer domain,
(b) a second fusion polypeptide comprising a first part of an antigen binding domain and a spacer domain, wherein
the spacer domain is a polypeptide and comprises at least 25 amino acid residues, and
wherein the second part of the antigen binding domain is fused either directly or via a third peptide linker to the C-terminus of the spacer domain or is present in form of a light chain, and
(c) a third ectodomain of said TNF ligand family member or a fragment thereof that is fused either directly or via a fourth peptide linker to
either the C-terminus of the second ectodomain of said TNF ligand family member in the first fusion polypeptide or to the C-terminus of the spacer domain in the second fusion polypeptide, or
in case the second part of the antigen binding domain is fused to the C-terminus of the spacer domain of the second fusion protein, to the C-terminus of the second ectodomain of said TNF ligand family member in the first fusion polypeptide,
wherein the spacer domain of the first fusion polypeptide and the spacer domain of the second fusion polypeptide are associated covalently to each other by a disulfide bond.

2. The TNF family ligand trimer-containing antigen binding molecule of claim 1, wherein the first part of the antigen binding domain comprises an antibody heavy chain variable domain and the second part of the antigen binding domain comprises an antibody light chain variable domain or vice versa.

3. The TNF family ligand trimer-containing antigen binding molecule of claim 1 or 2, wherein the first part of the antigen binding domain is an antibody heavy chain Fab fragment and the second part of the antigen binding domain is an antibody light chain Fab fragment or vice versa.

4. The TNF family ligand trimer-containing antigen binding molecule of any one of claims 1 to 3, wherein the spacer domain comprises an antibody hinge region or a fragment thereof, an antibody CH2 domain, and an antibody CH3 domain or a fragment thereof.

5. The TNF family ligand trimer-containing antigen binding molecule of any one of claims 1 to 4, wherein the spacer domain of the first fusion polypeptide and the spacer domain of the second fusion polypeptide comprise modifications promoting the association of the first and second fusion polypeptide.

6. The TNF family ligand trimer-containing antigen binding molecule of any one of claims 1 to 5, wherein the spacer domain comprises an antibody hinge region or a fragment thereof and an IgG1 Fc domain.

7. The TNF family ligand trimer-containing antigen binding molecule of any one of claims 1 to 6, wherein the IgG1 Fc domain comprises amino acid substitutions L234A, L235A and P329G (numbering according to Kabat EU index).

8. The TNF family ligand trimer-containing antigen binding molecule of any one of claims 1 to 7, wherein the TNF ligand family member is 4-1BBL.

9. The TNF family ligand trimer-containing antigen binding molecule of any one of claims 1 to 8, wherein the ectodomain of the TNF ligand family member comprises the amino acid sequence selected from the group consisting of SEQ ID NO:1, SEQ ID NO: 2, SEQ ID NO: 3, SEQ ID NO: 4, SEQ ID NO: 5, SEQ ID NO: 6, SEQ ID NO: 7 and SEQ ID NO: 8, particularly the amino acid sequence of SEQ ID NO: 1 or SEQ ID NO: 5.

10. The TNF family ligand trimer-containing antigen binding molecule of any one of claims 1 to 9, wherein the antigen binding domain is capable of specific binding to a tumor associated antigen.

11. The TNF family ligand trimer-containing antigen binding molecule of any one of claims 1 to 10, wherein the antigen binding domain is capable of specific binding to Fibroblast Activation Protein (FAP) or CD19.

12. The TNF family ligand trimer-containing antigen binding molecule of any one of claims 1 to 11, wherein the antigen binding domain capable of specific binding to FAP comprises
(a) a heavy chain variable region (V_{H}FAP) comprising (i) CDR-H1 comprising the amino acid sequence of SEQ ID NO: 9, (ii) CDR-H2 comprising the amino acid sequence of SEQ ID NO: 10, and (iii) CDR-H3 comprising the amino acid sequence of SEQ ID NO: 11, and a light chain variable region (V_{L}FAP) comprising (iv) CDR-L1 comprising the amino acid sequence of SEQ ID NO: 12, (v) CDR-L2 comprising the amino acid sequence of SEQ ID NO: 13, and (vi) CDR-L3 comprising the amino acid sequence of SEQ ID NO: 14, or
(b) a heavy chain variable region (V_{H}FAP) comprising (i) CDR-H1 comprising the amino acid sequence of SEQ ID NO: 15, (ii) CDR-H2 comprising the amino acid sequence of SEQ ID NO: 16, and (iii) CDR-H3 comprising the amino acid sequence of SEQ ID NO: 17, and a a light chain variable region (V_{L}FAP) comprising (iv) CDR-L1 comprising the amino acid sequence of SEQ ID NO: 18, (v) CDR-L2 comprising the amino acid sequence of SEQ ID NO: 19, and (vi) CDR-L3 comprising the amino acid sequence of SEQ ID NO: 20.

13. The TNF family ligand trimer-containing antigen binding molecule of any one of claims 1 to 14, wherein the antigen binding domain capable of specific binding to CD19 comprises
(a) a heavy chain variable region (V_{H}CD19) comprising (i) CDR-H1 comprising the amino acid sequence of SEQ ID NO: 25, (ii) CDR-H2 comprising the amino acid sequence of SEQ ID NO: 26, and (iii) CDR-H3 comprising the amino acid sequence of SEQ ID NO: 27, and a light chain variable region (V_{L}CD19) comprising (iv) CDR-L1 comprising the amino acid sequence of SEQ ID NO: 28, (v) CDR-L2 comprising the amino acid sequence of SEQ ID NO: 29, and (vi) CDR-L3 comprising the amino acid sequence of SEQ ID NO: 30, or
(b) a heavy chain variable region (V_{H}CD19) comprising (i) CDR-H1 comprising the amino acid sequence of SEQ ID NO: 31, (ii) CDR-H2 comprising the amino acid sequence of SEQ ID NO: 32, and (iii) CDR-H3 comprising the amino acid sequence of SEQ ID NO: 33, and a a light chain variable region (V_{L}CD19) comprising (iv) CDR-L1 comprising the amino acid sequence of SEQ ID NO: 34, (v) CDR-L2 comprising the amino acid sequence of SEQ ID NO: 35, and (vi) CDR-L3 comprising the amino acid sequence of SEQ ID NO: 36.

14. Isolated nucleic acid encoding the TNF family ligand trimer-containing antigen binding molecule of any one of claims 1 to 13.

15. A host cell comprising the nucleic acid of claim 14.

16. A pharmaceutical composition comprising the TNF family ligand trimer-containing antigen binding molecule of any one of claims 1 to 13 and a pharmaceutically acceptable excipient.

17. A method of treating an individual having cancer comprising administering to the individual an effective amount of the TNF family ligand trimer-containing antigen binding molecule of any one of claims 1 to 13 or the pharmaceutical composition of claim 16.
